(19) United States
(12) Patent Application Publication
(10) Pub. No.: US 2024/0011000 A1
(43) Pub. Date: Jan. 11, 2024

(54) RECOMBINANT PROTEINS WITH INCREASED SOLUBILITY AND STABILITY

(71) Applicant: BOARD OF REGENTS, THE UNIVERSITY OF TEXAS SYSTEM, Austin, TX (US)

(72) Inventors: Andrew ELLINGTON, Austin, TX (US); Inyup PAIK, Austin, TX (US); Andre MARANHAO, Austin, TX (US); Sanchita BHADRA, Austin, TX (US); David WALKER, Austin, TX (US); Phuoc NGO, Austin, TX (US); Daniel DIAZ, Austin, TX (US)

(21) Appl. No.: 18/044,909

(22) PCT Filed: Sep. 15, 2021
(86) PCT No.: PCT/US2021/050377

§ 371 Date: Mar. 10, 2023

Related U.S. Application Data
(60) Provisional application No. 63/078,621, filed on Sep. 15, 2020.
(60) Provisional application No. 63/168,557, filed on Mar. 31, 2021.

(51) Int. Cl. C12N 9/12; C12Q 1/70

(52) U.S. Cl. CPC C12N 9/1247; C12N 9/127; C12Y 207/07007; C12Y 207/07049; C12Q 1/701

(57) ABSTRACT

Disclosed are recombinant fusion proteins comprising a villin headpiece HP47 domain and a heterologous protein domain, such as, for example, a nucleic acid polymerase. Also disclosed are nucleic acids (e.g., DNA constructs) encoding the fusion protein, expression vectors and recombinant host cells for expression of the fusion protein, and methods of using the recombinant fusion proteins.

### REFERENCE TO RELATED APPLICATIONS

The present application claims the priority benefit of U.S. provisional application No. 63/078,621, filed Sep. 15, 2020, and U.S. provisional application No. 63/168,557, filed Mar. 31, 2021, the entire contents of each of which is incorporated herein by reference.

### STATEMENT REGARDING FEDERALLY SPONSORED RESEARCH

This invention was made with government support under Grant No. R01 EB027202 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Sep. 12, 2021, is named UTFBP1265WO_ST25.txt and is 39.6 kilobytes in size.

### BACKGROUND

### 1. Field

The present disclosure relates generally to the fields of molecular biology, cell biology, biochemistry, research, medicine, and diagnostics. More particularly, it concerns improved thermostable polymerases and methods of their use.

### 2. Description of Related Art

Despite the fact that strand-displacing activity is of great utility for a variety of applications, including isothermal amplification assays, there are relatively few strand-displacing DNA polymerases. In particular, the thermotolerant DNA polymerase from Geobacillus stearothermophilus (previously Bacillus stearothermophilus), Bst DNA polymerase (Bst DNAP), is used in a variety of assays, including loop-mediated isothermal amplification. However, despite its wide use, its properties remain open to improvement, as has been demonstrated by a variety of engineering efforts, including the identification of point mutations that impact its robustness, strand-displacement capabilities, and nascent reverse transcriptase activity.

### SUMMARY

In one embodiment, provided herein are recombinant proteins comprising, from N-terminus to C-terminus, an N-terminal stabilizer domain, optionally a first linker region, and a heterologous protein domain, wherein the N-terminal stabilizer domain comprises a sequence at least 90% identical to either SEQ ID NO: 2 or 3. In some aspects, the N-terminal stabilizer domain comprises a sequence at least 91%, 92%, 93%, 94%, 95%, 96%, 97%, 98%, or 99% identical to either SEQ ID NO: 2 or 3. In some aspects, the N-terminal stabilizer domain consists of a sequence at least 91%, 92%, 93%, 94%, 95%, 96%, 97%, 98%, or 99% identical to either SEQ ID NO: 2 or 3. In some aspects, the N-terminal stabilizer domain improves the folding, solubility, stability, and/or substrate binding of the recombinant protein and is derived, by way of one or more amino acid substitutions, deletions or insertions, from the polypeptide sequence SEQ ID NO: 2 or 3. In some aspects, the N-terminal stabilizer domain comprises a sequence identical to either SEQ ID NO: 2 or 3. In some aspects, the N-terminal stabilizer domain consists of a sequence identical to either SEQ ID NO: 2 or 3.

In some aspects, the N-terminal stabilizer domain has a negatively charged surface and a positively charged surface. In some aspects, the N-terminal stabilizer domain comprises at least one substitution that enhances the positivity of the positively charged surface of the domain. In some aspects, the N-terminal stabilizer domain comprises at least one substitution at a position corresponding to K9, A20, N31, N39, or E43 of SEQ ID NO: 2. In some aspects, the N-terminal stabilizer domain comprises at least one substitution to a positively charged amino acid at a position corresponding to K9, A20, N31, N39, or E43 of SEQ ID NO: 2. In some aspects, the N-terminal stabilizer domain comprises a substitution to a lysine at a position corresponding to K9 of SEQ ID NO: 2. In some aspects, the N-terminal stabilizer domain comprises a substitution to a lysine at a position corresponding to A20 of SEQ ID NO: 2. In some aspects, the N-terminal stabilizer domain comprises a substitution to an arginine at a position corresponding to N31 of SEQ ID NO: 2. In some aspects, the N-terminal stabilizer domain comprises a substitution to a lysine at a position corresponding to N39 of SEQ ID NO: 2. In some aspects, the N-terminal stabilizer domain comprises a substitution to a lysine at a position corresponding to E43 of SEQ ID NO: 2. In some aspects, the N-terminal stabilizer domain comprises at least two substitutions selected from A20K, N31R, N39K, and E43K. In some aspects, the N-terminal stabilizer domain comprises at least three substitutions selected from K9D, A20K, N31R, N39K, and E43K. In some aspects, the N-terminal stabilizer domain comprises N31R, N39K, and E43K substitutions. In some aspects, the N-terminal stabilizer domain comprises A20K, N31R, N39K, and E43K substitutions. In some aspects, the N-terminal stabilizer domain comprises K9D, N31R, N39K, and E43K substitutions.

In various aspects of the present embodiments, the heterologous protein domain has enzymatic function. For example, the heterologous protein domain may have protease function, nuclease function, transposase function, or polymerase function. In various aspects, the recombinant protein is stored in a storage buffer or a reaction buffer.

In various aspects of the present embodiments, the heterologous protein domain is a nucleic acid polymerase. The nucleic acid polymerase may be, for example, a DNA polymerase. Such a DNA polymerase may be a DNA-dependent DNA polymerase, an RNA-dependent DNA polymerase, or both a DNA-dependent DNA polymerase and an RNA-dependent DNA polymerase. In various aspects, the nucleic acid polymerase is a Bst DNA polymerase, large fragment (e.g., Bst LF, SEQ ID NO: 4, with or without the initial methionine), a Taq DNA polymerase (e.g., Klentaq, SEQ ID NO: 5, with or without the initial methionine), or a Bst-Taq chimera (e.g., V5.9, SEQ ID NO: 6, in particular amino acids 17-568 of SEQ ID NO: 6). In some aspects, the nucleic acid polymerase comprises a sequence at least 95% identical to SEQ ID NO: 4. In some aspects, the nucleic acid polymerase has thermostable polymerase activity and is derived, by way of one or more amino acid substitutions, deletions or insertions, from the polypeptide sequence of SEQ ID NO: 4. In some aspects, the nucleic acid polymerase comprises at least one substitution that enhances the thermostability of the nucleic acid polymerase. In some aspects, the nucleic acid polymerase comprises at least one mutation relative to the sequence of SEQ ID NO: 4. In some aspects, the nucleic acid polymerase comprises at least one substitution at a position corresponding to V191, S371, T493, A552, or R562 of SEQ ID NO: 4. In some aspects, the nucleic acid polymerase comprises a substitution to a leucine at a position corresponding to V191 of SEQ ID NO: 4. In some aspects, the nucleic acid polymerase comprises a substitution to an aspartic acid at a position corresponding to 5371 of SEQ ID NO: 4. In some aspects, the nucleic acid polymerase comprises a substitution to an asparagine at a position corresponding to T493 of SEQ ID NO: 4. In some aspects, the nucleic acid polymerase comprises a substitution to a glycine at a position corresponding to A552 of SEQ ID NO: 4. In some aspects, the nucleic acid polymerase comprises a substitution to a valine at a position corresponding to R562 of SEQ ID NO: 4. In some aspects, the nucleic acid polymerase comprises at least two substitutions selected from V191L, S371D, T493N, A552G, and R562V. In some aspects, the nucleic acid polymerase comprises at least three substitutions selected from V191L, S371D, T493N, A552G, and R562V. In some aspects, the nucleic acid polymerase comprises T493N, A552G, and R562V substitutions. In some aspects, the nucleic acid polymerase comprises S371D, T493N, and A552G substitutions.

In some aspects, the recombinant protein comprises a sequence at least 90%, 91%, 92%, 93%, 94%, 95%, 96%, 97%, 98%, or 99% identical to amino acids 11-644 of SEQ ID NO: 19. In some aspects, the recombinant protein has increased thermostability and polymerase activity and is derived, by way of one or more amino acid substitutions, deletions or insertions, from the polypeptide sequence SEQ ID NO: 19. In some aspects, the recombinant protein comprises a sequence identical to amino acids 11-644 of SEQ ID NO: 19. In some aspects, the recombinant protein consists of a sequence identical to amino acids 1-644 of SEQ ID NO: 19. In some aspects, the recombinant protein consists of a sequence identical to amino acids 11-644 of SEQ ID NO: 19.

In some aspects, the recombinant protein comprises at least one substitution at a position corresponding to K19, A30, N41, N49, or E53 of SEQ ID NO: 19. In some aspects, the recombinant protein comprises at least one substitution to a positively charged amino acid at a position corresponding to K19, A30, N41, N49, or E53 of SEQ ID NO: 19. In some aspects, the recombinant protein comprises a substitution to a lysine at a position corresponding to K19 of SEQ ID NO: 19. In some aspects, the recombinant protein comprises a substitution to a lysine at a position corresponding to A30 of SEQ ID NO: 19. In some aspects, the recombinant protein comprises a substitution to an arginine at a position corresponding to N41 of SEQ ID NO: 19. In some aspects, the recombinant protein comprises a substitution to a lysine at a position corresponding to N49 of SEQ ID NO: 19. In some aspects, the recombinant protein comprises a substitution to a lysine at a position corresponding to E53 of SEQ ID NO: 19. In some aspects, the recombinant protein comprises at least two substitutions selected from A30K, N41R, N49K, and E53K. In some aspects, the recombinant protein comprises at least three substitutions selected from K19D, A30K, N41R, N49K, and E53K. In some aspects, the recombinant protein comprises N41R, N49K, and E53K substitutions. In some aspects, the recombinant protein comprises K19D, N41R, N49K, and E53K substitutions. In some aspects, the recombinant protein comprises A30K, N41R, N49K, and E53K substitutions. In some aspects, the recombinant protein comprises at least one substitution at a position corresponding to V243, S423, T545, A604, or R614 of SEQ ID NO: 19. In some aspects, the recombinant protein comprises a substitution to a leucine at a position corresponding to V243 of SEQ ID NO: 19. In some aspects, the recombinant protein comprises a substitution to an aspartic acid at a position corresponding to S423 of SEQ ID NO: 19. In some aspects, the recombinant protein comprises a substitution to an asparagine at a position corresponding to T545 of SEQ ID NO: 19. In some aspects, the recombinant protein comprises a substitution to a glycine at a position corresponding to A604 of SEQ ID NO: 19. In some aspects, the recombinant protein comprises a substitution to a valine at a position corresponding to R614 of SEQ ID NO: 19. In some aspects, the recombinant protein comprises at least two substitutions selected from V243L, S423D, T545N, A604G, and R614V. In some aspects, the recombinant protein comprises at least three substitutions selected from V243L, S423D, T545N, A604G, and R614V. In some aspects, the recombinant protein comprises T545N, A604G, and R614V substitutions. In some aspects, the recombinant protein comprises S423D, T545N, and A604G substitutions. In some aspects, the recombinant protein comprises A30K, N41R, N49K, E53K, S423D, T545N, and A604G substitutions. In some aspects, the recombinant protein comprises N41R, N49K, E53K, S423D, T545N, and A604G substitutions.

In various aspects of the present embodiments, the nucleic acid polymerase lacks 5′ to 3′ exonuclease activity. In various aspects, the nucleic acid polymerase is capable of replicating DNA and/or RNA in an isothermal amplification reaction. In certain aspects, the isothermal amplification reaction is loop-mediated isothermal amplification (LAMP), strand displacement amplification (SDA), polymerase spiral reaction (PSR), helicase dependent amplification (HDA), or rolling circle amplification (RCA).

In various aspects of the present embodiments, the first linker region is a flexible linker or a cleavable linker. In some aspects, the first linker region comprises a sequence according to any one of SEQ ID NOs: 8-17. In certain aspects, the first linker region comprises a sequence according to SEQ ID NO: 8.

In various aspects of the present embodiments, the recombinant proteins further comprise an augmentative protein domain positioned N-terminally relative to the N-terminal stabilizer domain. In some aspects, the augmentative protein domain is a DNA binding protein. The DNA binding protein may be a single-stranded DNA binding protein, such as, for example, the single-stranded DNA binding protein is extreme thermostable single-stranded DNA binding protein (ET SSB), E. coli recA, T7 gene 2.5 product, phage lambda RedB, or Rac prophage RecT.

In various aspects of the present embodiments, the recombinant proteins further comprise a second linker region positioned between the augmentative protein domain and the N-terminal stabilizer domain. In some aspects, the second linker region is a flexible linker or a cleavable linker. In some aspects, the second linker region comprises a sequence according to any one of SEQ ID NOs: 8-17. In some aspects, the second linker region comprises a sequence according to SEQ ID NO: 8.

In various aspects of the present embodiments, the recombinant proteins further comprise an N-terminal purification tag, such as, for example, a His-tag.

In one embodiment, provided herein are compositions comprising a recombinant protein of any one of the present embodiments. In some aspects, the compositions further comprise a storage buffer or a reaction buffer. In some aspects, the compositions further comprise at least one oligonucleotide. In some aspects, the compositions are lyophilized.

In one embodiment, provided herein are nucleic acids encoding a recombinant protein of any one of the present embodiments.

In one embodiment, provided herein are host cells comprising a nucleic acid encoding a recombinant protein of any one of the present embodiments. In some aspects, the nucleic acid is codon optimized based on the codon usage of the host cell.

In one embodiment, provided herein are kits comprising a recombinant protein of any one of the present embodiments.

In one embodiment, provided herein are kits comprising a composition of any one of the present embodiments.

In one embodiment, provided herein are methods of amplifying a nucleic acid, the methods comprising exposing a sample that may contain a target nucleic acid to a buffer solution comprising oligonucleotide primers that are capable of hybridizing to the target nucleic acid and amplifying the target nucleic acid using a nucleic acid polymerase of any one of the present embodiments. In some aspects, the amplification uses an isothermal amplification reaction. In various aspects, the isothermal amplification reaction is loop-mediated isothermal amplification (LAMP), strand displacement amplification (SDA), polymerase spiral reaction (PSR), helicase dependent amplification (HDA), or rolling circle amplification (RCA). The target nucleic acid may be DNA or RNA, and the DNA or RNA may comprise modified nucleotides. When the target nucleic acid is an RNA, the amplifying may be performed without a separate reverse transcription step and/or without a separate reverse transcriptase. The reverse transcription of the RNA may be performed by the nucleic acid polymerase of any one of the present embodiments. Alternatively, the amplifying may be performed in the presence of a dedicated reverse transcriptase. In some aspects, the reaction may be performed at take place at 20, 25, 30, 35, 40, 45, 50, 55, 60, 61, 62, 63, 64, 65, 66, 67, 68, 69, 70, 71, 72, 73, 74, 75, 76, 77, 78, 79, 80, 81, 82, 83, 84, 85, 90, or 95° C. In some aspects, the reaction is performed for no more than 6, 7, 8, 9, 10, 11, 12, 13, 14, or 15 minutes. In some aspects, the reaction is performed for 6-10 mM at 73 or 74° C. In some aspects, the sample comprises a chaotropic agent, such as, for example, guanidinium, ethanol, lithium, phenol, sodium dodecyl sulfate, thiourea, or urea. In some aspects, the sample is a urine sample. In some aspects, the sample comprises at least 50 mM guanidinium. In some aspects, the sample comprises at least 2 M guanidinium.

In one embodiment, provided herein are methods of diagnosing a subject with a disease, the method comprising carrying out the method of any one of the present embodiments, wherein the presence of a target nucleic acid indicates the presence of a disease in the subject. In some aspects, the disease is a virus, such as, for example, SARS-CoV-2. In some aspects, the methods take place in a single vessel.

In one embodiment, provided herein are methods for expression of a protein of interest comprising expressing in a host cell a nucleic acid molecule encoding the protein of interest fused to a N-terminal stabilizer domain having a negatively charged surface and a positively charged surface. In some aspects, the N-terminal stabilizer domain comprising a sequence at least 90% identical to either SEQ ID NO: 2 or 3. In some aspects, the N-terminal stabilizer domain comprises a sequence at least 91%, 92%, 93%, 94%, 95%, 96%, 97%, 98%, or 99% identical to either SEQ ID NO: 2 or 3. In some aspects, the N-terminal stabilizer domain consists of a sequence at least 91%, 92%, 93%, 94%, 95%, 96%, 97%, 98%, or 99% identical to either SEQ ID NO: 2 or 3. In some aspects, the N-terminal stabilizer domain comprises a sequence identical to either SEQ ID NO: 2 or 3. In some aspects, the N-terminal stabilizer domain consists of a sequence identical to either SEQ ID NO: 2 or 3. In various aspects, the methods are further defined as methods for enhancing the folding or solubility of the protein of interest or methods for enhancing the nucleic acid binding ability of the protein of interest. In various aspects, the methods are further defined as methods for enhancing specific activity of an enzyme. In various aspects, the methods are further defined as methods for enhancing thermal stability of an enzyme.

In one embodiment, provided herein are methods for expression of a protein of interest comprising expressing in a host cell a nucleic acid molecule encoding the protein of interest fused to a N-terminal stabilizer domain comprising a sequence at least 90% identical to either SEQ ID NO: 2 or 3. In some aspects, the N-terminal stabilizer domain comprises a sequence at least 91%, 92%, 93%, 94%, 95%, 96%, 97%, 98%, or 99% identical to either SEQ ID NO: 2 or 3. In some aspects, the N-terminal stabilizer domain consists of a sequence at least 91%, 92%, 93%, 94%, 95%, 96%, 97%, 98%, or 99% identical to either SEQ ID NO: 2 or 3. In some aspects, the N-terminal stabilizer domain comprises a sequence identical to either SEQ ID NO: 2 or 3. In some aspects, the N-terminal stabilizer domain consists of a sequence identical to either SEQ ID NO: 2 or 3. In various aspects, the methods are further defined as methods for enhancing the folding or solubility of the protein of interest or methods for enhancing the nucleic acid binding ability of the protein of interest. In various aspects, the methods are further defined as methods for enhancing specific activity of an enzyme. In various aspects, the methods are further defined as methods for enhancing thermal stability of an enzyme.

In various aspects of the present embodiments, the protein of interest has enzymatic function. For example, the protein of interest may have protease function, nuclease function, transposase function, or polymerase function.

In various aspects of the present embodiments, the protein of interest is a nucleic acid polymerase. The nucleic acid polymerase may be, for example, a DNA polymerase. Such a DNA polymerase may be a DNA-dependent DNA polymerase, an RNA-dependent DNA polymerase, or both a DNA-dependent DNA polymerase and an RNA-dependent DNA polymerase. The nucleic acid polymerase may function as a reverse transcriptase for chemically diverse nucleic acid templates. In various aspects, the nucleic acid polymerase is a Bst DNA polymerase, large fragment (e.g., Bst LF, SEQ ID NO: 4, with or without the initial methionine), a Taq DNA polymerase (e.g., Klentaq, SEQ ID NO: 5, with or without the initial methionine), or a Bst-Taq chimera (e.g., V5.9, SEQ ID NO: 6, in particular amino acids 17-568 of SEQ ID NO: 6). In some aspects, the protein of interest comprises a sequence at least 90%, 91%, 92%, 93%, 94%, 95%, 96%, 97%, 98%, or 99% identical to SEQ ID NO: 19. In some aspects, the protein of interest comprises a sequence identical to SEQ ID NO: 19. In some aspects, the protein of interest consists of a sequence identical to SEQ ID NO: 19. In some aspects, the nucleic acid encoding the protein of interest comprises a sequence at least 85%, 86%, 87%, 88%, 89%, 90%, 91%, 92%, 93%, 94%, 95%, 96%, 97%, 98%, or 99% identical to SEQ ID NO: 20. In some aspects, the nucleic acid encoding the protein of interest comprises a sequence identical to SEQ ID NO: 20.

In various aspects of the present embodiments, the nucleic acid polymerase lacks 5′ to 3′ exonuclease activity. In various aspects, the nucleic acid polymerase is capable of replicating DNA and/or RNA in an isothermal amplification reaction. In certain aspects, the isothermal amplification reaction is loop-mediated isothermal amplification (LAMP), strand displacement amplification (SDA), polymerase spiral reaction (PSR), or helicase dependent amplification (HDA).

In various aspects of the present embodiments, the nucleic acid molecule further encodes a first linker positioned between the N-terminal stabilizer domain and the protein of interest. In some aspects, the first linker region is a flexible linker or a cleavable linker. In some aspects, the first linker region comprises a sequence according to any one of SEQ ID NOs: 8-17. In certain aspects, the first linker region comprises a sequence according to SEQ ID NO: 8.

In various aspects of the present embodiments, the nucleic acid molecule further encodes an augmentative protein domain positioned N-terminally relative to the N-terminal stabilizer domain. In some aspects, the augmentative protein domain is a DNA binding protein. The DNA binding protein may be a single-stranded DNA binding protein, such as, for example, the single-stranded DNA binding protein is extreme thermostable single-stranded DNA binding protein (ET SSB), E. coli recA, T7 gene 2.5 product, phage lambda RedB, or Rac prophage RecT.

In various aspects of the present embodiments, the nucleic acid molecule further encodes a second linker region positioned between the augmentative protein domain and the N-terminal stabilizer domain. In some aspects, the second linker region is a flexible linker or a cleavable linker. In some aspects, the second linker region comprises a sequence according to any one of SEQ ID NOs: 8-17. In some aspects, the second linker region comprises a sequence according to SEQ ID NO: 8.

In various aspects of the present embodiments, the nucleic acid molecule further encodes an N-terminal purification tag, such as, for example, a His-tag.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B. Graphical representation of Br512 and the electrostatic force map of HP47. (FIG. 1A) Br512 was constructed by fusing HP47 with a GS linker to the N-terminus of Bst-LF. A His-Tag was added at the N-terminus of the new fusion protein to aid purification. (FIG. 1B) Models of HP47 electrostatic force using an Adaptive Poisson-Boltzmann Solver to identify surface charge. The charge designations are referenced in the bar at the bottom. Each graphic is the same model with different orientations rotated on the Y-Axis. Graphics were created in PyMol.

FIGS. 2A-D. Comparison of Br512, Bst-LF, and Bst 2.0 in LAMP-OSD assays of DNA templates. LAMP-OSD assays for human gapd gene were performed using 16 units of commercially sourced Bst 2.0 (FIG. 2A), 20 pm of in-house purified Bst-LF (FIG. 2B), or 20 pm of Br512 (FIGS. 2C and 2D) in indicated reaction buffers Amplification curves observed in real-time at 65° C. by measuring OSD fluorescence in reactions seeded with 600,000, 60,000, 6,000, 600, and 0 copies of gapd plasmid templates are depicted.

FIG. 3. Comparison of Br512 and Bst 2.0 in RT-LAMP-OSD assays for SARS-CoV-2 viral genomic RNA. Three SARS-CoV-2-specific RT-LAMP-OSD assays, NB, 6-Lamb, and Tholoth, targeting three different regions in the viral genomic RNA were performed using 20 pm of Br512 (left panels) in G6D buffer or 16 units of commercially sourced Bst 2.0 (right panels) in isothermal buffer. Images of OSD fluorescence taken at assay endpoint (after 60 min of amplification at 65° C. followed by cooling to room temperature) are depicted.

FIGS. 4A-C. Comparison of Br512, Bst-LF, and Bst 2.0 in RT-LAMP-OSD assays for SARS-CoV-2 genomic RNA. Three SARS-CoV-2-specific RT-LAMP-OSD assays, NB (FIG. 4A), Tholoth (FIG. 4B), and 6-Lamb (FIG. 4C), were operated using 20 pm of in-house purified Bst-LF, 16 units of commercially sourced Bst 2.0, or 20 pm of Br512 in G6D, isothermal, and G6D reaction buffers, respectively. OSD fluorescence measured at assay endpoint in reactions seeded with 3,000, 300, or 0 copies of SARS-CoV-2 viral genomic RNA templates are depicted. Assay replicates in each panel are numbered 1 through 5. Real-time amplification kinetics of each reaction are detailed in FIGS. 10A-I.

FIGS. 5A-B. Comparison of Br512 and Bst2.0 SARS-CoV-2 RT-LAMP-OSD assays in saliva. (FIG. 5A) Comparison of Br512 and Bst 2.0 DNA polymerase activity in LAMP-OSD assays of endogenous DNA templates saliva. Duplicate LAMP-OSD assays with (+) or without (−−) primers for human gapd gene were performed either with 16 units of Bst 2.0 in isothermal buffer (NEB) or with 20 pm of Br512 in G6B buffer. Assays were seeded with 3 μL of water (−−) or human saliva (+) heated for 10 min at 95° C. Images of OSD fluorescence taken at assay endpoint (after 60 min of amplification at 65° C. followed by cooling to room temperature) are depicted. (FIG. 5B) Duplicate multiplex RT-LAMP-OSD assays containing primers and OSD probes for both NB and 6-Lamb SARS-CoV-2 assays were executed with indicated amounts of either Bst 2.0 in isothermal buffer (NEB) or Br512 in G6D buffer. Assays were seeded with indicated copies of SARS-CoV-2 virions in the presence of 3 μL of human saliva heated for 10 min at 95° C. Some assays performed using Br512 also contained the RNase inhibitor, Superase. In. Images of OSD fluorescence taken at assay endpoint after 60 min of amplification at 65° C. followed by cooling to room temperature are depicted.

FIG. 6. Assessment of lyophilized Br512 multiplex SARS-CoV-2 RT-LAMP-OSD assays. Lyophilized multiplex RT-LAMP-OSD assays prepared with 20 or 30 picomoles of glycerol-free Br512 enzymes and primers and OSD probes for both NB and 6-Lamb assays were tested with indicated copies of SARS-CoV-2 genomic RNA. Images of OSD fluorescence taken after 60 min of amplification at 65° C. followed by cooling to room temperature are depicted.

FIG. 7. Effect of varying amounts of Br512 on LAMP-OSD of DNA templates. Indicated amounts of Br512 were compared with indicated amounts of in-house purified Bst-LF and commercially sourced Bst 2.0 in human gapd gene-specific LAMP-OSD assays operated in 1× isothermal buffer (NEB). Reactions were seeded with either 6000 copies of gapd plasmid template or with no specific templates (NTC) Amplification curves generated by real-time measurement of OSD fluorescence at 65° C. are depicted.

FIG. 8. Comparison of Br512, Bst-LF, and Bst 2.0 in LAMP assays of DNA templates read using EvaGreen intercalating dye. LAMP assays for human gapd gene were operated using Bst 2.0, Bst-LF, or Br512 in indicated reaction buffers Amplification curves observed in real-time at 65° C. by measuring EvaGreen fluorescence in reactions seeded with 600,000, 60,000, 6,000, 600, and 0 copies of gapd plasmid templates are depicted. LAMP amplicons were analyzed using the ‘melt curve analysis’ on LightCycler 96 real-time PCR machine and resulting melting peaks are indicated in the corresponding colored traces.

FIG. 9. Bst 2.0 Tholoth RT-LAMP-OSD assay executed in G6D buffer. Tholoth RT-LAMP-OSD assays for SARS-CoV-2 were operated using Bst 2.0 in G6D reaction buffer. OSD fluorescence measured in real-time during assay incubation at 65° C. are depicted within gray shaded boxes for reactions seeded with 3,000, 300, or 0 copies of SARS-CoV-2 genomic RNA templates. Post-amplification phase OSD signal measured at 37° C. before and after a 1 min DNA denaturation step at 95° C. (at min 111) are depicted within the darker shaded regions.

FIGS. 10A-I. Comparison of Br512, Bst-LF, and Bst 2.0 in RT-LAMP-OSD assays for SARS-CoV-2 genomic RNA. Three SARS-CoV-2-specific RT-LAMP-OSD assays, NB (FIGS. 10A, D, and G), Tholoth (FIGS. 10B, E, and H), and 6-Lamb (FIGS. 10C, F, and I), were operated using 20 pm of in-house purified Bst-LF (FIGS. 10A, B, and C), 16 units of commercially sourced Bst 2.0 (FIGS. 10D, E, and F), or 20 pm of Br512 (FIGS. 10G, H, and I) in indicated reaction buffers. Amplification kinetics at 65° C. observed in real-time by measuring OSD fluorescence in reactions seeded with 3,000, 300, or 0 copies of SARS-CoV-2 viral genomic RNA templates are depicted within gray shaded boxes. Post-amplification OSD signal measured at 37° C. before and after a 1 min DNA denaturation step at 95° C. (at about min 108) are depicted within the darker shaded regions. Assay replicates in each panel are numbered 1 through 5.

FIG. 11. Comparison of Br512 and Bst 2.0 DNA polymerase activity in LAMP-OSD assays of endogenous DNA templates saliva. Duplicate LAMP-OSD assays with (+) or without (−−) primers for human gapd gene were performed either with 16 units of Bst 2.0 in isothermal buffer (NEB) or with 20 pm of Br512 in G6B buffer. Assays were seeded with 3 μL of water (−−) or human saliva (+) heated for 10 min at 95° C. Images of OSD fluorescence taken at assay endpoint (after 60 min of amplification at 65° C. followed by cooling to room temperature) are depicted.

FIGS. 12A-B. Comparison of Br512 and Bst2.0 SARS-CoV-2 multiplex RT-LAMP-OSD assays. Duplicate multiplex RT-LAMP-OSD assays containing primers and OSD probes for both NB and 6-Lamb assays were executed with either 20 pm of Br512 in G6D buffer (FIG. 12A) or 16 units of Bst 2.0 in isothermal buffer (NEB) (FIG. 12B). Assays were seeded with indicated copies of SARS-CoV-2 genomic RNA and amplification kinetics at 65° C. observed in real-time by measuring OSD fluorescence are depicted in gray shaded boxes as 3000 copies, 300 copies, 100 copies, and 0 copies. Post-amplification OSD signal measured at 37° C. before and after a 1 min DNA denaturation step at 95° C. are depicted in the darker shaded regions.

FIGS. 13A-C. Comparison of Br512 activity in different LAMP-OSD assay buffers. LAMP-OSD assays with the human gapd gene were carried out with Br512 in the indicated reaction buffers (FIG. 13A is G1B buffer; FIG. 13B is G2A buffer; FIG. 13C is G3A buffer). Amplification curves were observed in real-time by measuring OSD fluorescence at ° C. in reactions seeded with 600,000, 60,000, 6,000, 600, and 0 copies of gapd plasmid templates.

FIG. 14. A flowchart of simple two-step Br512 purification. Simple two-step purification procedures are shown in the flowchart. E. coli BL21 cell expressed Br512 was initially purified with Ni-NTA based immobilized metal affinity chromatography (IMAC), and further purified with heparin column based FPLC. A detailed purification protocol is described in Example 1.

FIGS. 15A-D. Br512 MutCompute mutations and their effect on enzyme thermal stability. (FIG. 15A) Table listing Br512 stabilizing amino acid substitutions suggested by MutCompute. Wild type (WT) vs predicted (Pred) amino acid mutations (column 2; positions in reference to PDB database ID; STAN) were designated as Mut1 to Mut10 according to their predicted priorities. The calculated probabilities of the wild type and predicted amino acids at each position are indicated in columns 3 and 4, respectively. (FIGS. Effect of thermal challenge on wildtype and triple mutant MutCompute variants of Br512. Identical GAPDH LAMP assays assembled using the same amount of indicated enzymes were subjected to either no heat challenge (FIG. 15B), 3 min at 75° C. (FIG. 15C), or sec at 80° C. (FIG. 15D) prior to real time measurement of GAPDH DNA amplification kinetics at 65° C. Representative amplification curves generated by measuring increases in EvaGreen dye fluorescence (Y-axis) over time (X-axis; time in hh:mm:ss) are depicted.

FIGS. 16A-C. Effect of supercharged villin headpiece on Br512 thermostability. (FIG. 16A) The Villin headpiece (vHP47) amino acid sequence and its corresponding supercharging mutations. Neutral, negatively charged, and positively charged amino acids are depicted by green, red, and blue letter designations, respectively. (FIG. 16B) Surface charge models of wildtype (wt) vHP47 domain and its supercharged variants generated as described in FIG. 1. A total eight amino acids of vHP47 designated as SC1-8 were mutated into either negatively (SC1,2,3,4) (Aspartate D/Glutamate E) or positively charged amino acids (SC5,6,7,8) (Lysine; K/Arginine; R). (FIG. 16C) Effect of thermal challenge on triple and quadruple positively supercharged mutants of Br512. Identical GAPDH LAMP assays assembled using the same amount of indicated enzymes were subjected to either no heat challenge (top panel), 3 min at 75° C. (middle panel), or 30 sec at 80° C. (bottom panel) prior to real time measurement of GAPDH DNA amplification kinetics at 65° C. Representative amplification curves generated by measuring increases in EvaGreen dye fluorescence (Y-axis) over time (X-axis; time in hh:mm:ss) are depicted as blue (Br512 wild type), burnt orange (SC5,6,7,8), gray (SC6,7,8), and yellow (SC5,7,8) traces. The effect of various single, double, and triple mutations are shown in FIGS. 23 and 24.

FIGS. 17A-D. Effect of combining MutCompute and supercharging mutations on Br512 thermal stabilities. Identical GAPDH LAMP assays assembled using either wildtype (wt), Supercharged-villin headpiece (SC), MutCompute (Mut), or combined SC+Mut Br512 variants were subjected to either no heat challenge (FIG. 17A), 3 min at 75° C. (FIG. 17B), 30 sec at 80° C. (FIG. 17C), or 30 sec at 82° C. (FIG. 17D) prior to real time measurement of GAPDH DNA amplification kinetics at 65° C. Threshold cycle (Cₜ) values for amplification of 20 pg (6×10⁷ copies) GAPDH DNA templates were calculated using the LightCycler 96 software and plotted as bar graphs. Time to reach the threshold cycles (Cₜ) are shown as bar graphs, in minutes. Standard deviation in Cₜ values calculated from three replicate experiments is depicted as error bars. N/D=Amplification not detected.

FIGS. 18A-C. Comparison of Br512 variants in high temperature LAMP assays. (FIGS. 18A-B) Identical GAPDH LAMP assays were assembled using the same amounts of either wildtype or mutant Br512 variants, and incubated at 74° C. for up to two hours. Amplification kinetics of GAPDH DNA templates were determined by real time measurement of EvaGreen dye fluorescence and the threshold cycles (Cₜ) for amplification of 20 pg (6×10⁷ copies) GAPDH DNA templates were calculated using the Lightcyler 96 software. (FIG. 18C) (Left panel) High temperature (74° C.) LAMP assay amplification results from 600,000; 6,000; and 0 copies of GAPDH DNA templates are shown. Dotted lines indicate traces from 0 templates (Right panel) Calculated threshold cycles (Cₜ) in minutes from LAMP assays shown in (FIG. 18C). Representative changes in fluorescence intensities (Y-axis) over time are depicted as amplification curves (FIGS. 18A and C) while average Cₜ values from three replicate experiments are plotted as bar graphs (FIG. 18B; 1ΔCt=4 mins; N/D=Amplification Not Detected; Error Bar=S.D; n=3).

FIG. 19. Initial evaluation of computationally predicted substitutions on Br512 (Bst-LF) activity. LAMP assays were carried out with a 20 pg (6×10⁷ copies) of GAPDH DNA template to assess the effect of the individual mutations suggested by Mutcompute on Br512 activity. Amplification was observed by EvaGreen dye fluorescence change (Y-axis) over time of incubation (X-axis) at 65° C.

FIG. 20. Heat challenge LAMP assay with computationally predicted single amino acid substitutions. LAMP assays assembled with wildtype (wt) or Mutcompute calculated Br512 variants (Mut1 to 5) were subjected to indicated thermal challenges (top panel: no thermal challenge; middle panel: 3 min at 75° C.; lower panel: 30 sec at 80° C.) prior to real time measurement of DNA amplification during continuous incubation at 65° C. Amplification kinetics was determined by measuring EvaGreen fluorescence (Y-axis) over incubation time (X-axis; hh:mm:ss).

FIG. 21. Heat challenge LAMP assay with double mutation Br512 variants. Activities of wild type (blue traces) and the various double mutant Mutcompute Br512 variants (orange traces) were compared in identical LAMP assays containing 20 pg (6×10⁷ copies) of GAPDH DNA templates that were subjected to indicated thermal challenges (top panel: no thermal challenge; middle panel: 3 min at 75° C.; lower panel: 30 sec at 80° C.) prior to real time measurement of DNA amplification at 65° C. Representative amplification curves determined by measuring EvaGreen fluorescence (Y-axis) over incubation time (X-axis; hh:mm:ss) are depicted.

FIG. 22. Threshold cycle (Ct) analysis of triple Mutcompute variants. GAPDH LAMP assay results shown in FIGS. 15B-D were further quantified with Cₜ values in minutes. Threshold cycles for amplification of 20 pg (6×10⁷ copies) GAPDH DNA templates were calculated using the Lightcycler96 software (Roche). Lower Cₜ indicates faster amplification. Upper panel: Cₜ values for No Heat LAMP, Middle panel: Cₜ values for 75° C. 3 min heat challenge LAMP, Lower panel: Cₜ values for 80° C. 30 sec heat challenge LAMP. Error bar=S.D., n=2 for No Heat, n=3 for 75° C. and 80° C. heat challenge LAMP (Y-axis: Cₜ in minutes).

FIG. 23. Alignment of vHP47 sequence and its conserved amino acids among its orthologues. Amino acid sequence of villin headpiece vHP47 (SEQ ID NO: 2) was blasted at blast.ncbi.nlm nih.gov/Blast.cgi with blastp (protein-protein BLAST) algorithm. Top 100 hit sequences were compared using NCBI Multiple Sequences Alignment Viewer Amino acids that are identical to consensus sequence were highlighted. Mean hydrophobicity is shown as a bar graph. Top row: consensus amino acid sequence (SEQ ID NO: 21).

FIG. 24. Heat challenge of single mutation supercharged Br512 variants. Identical LAMP assays assembled using wild type (blue) or mutant (orange and gray) Br512 were heat challenged at either 75° C. for 3 min or 80° C. for 30 sec prior to determining amplification kinetics at 65° C. Representative amplification curves determined by measuring EvaGreen fluorescence (Y-axis) over incubation time (X-axis; hh:mm:ss) are depicted.

FIG. 25. Heat challenge of supercharged double and triple mutation Br512 variants. Identical GAPDH LAMP assays assembled using either double mutation variants (Left panels) or triple mutation variants (right panels) were subjected to indicated heat challenges prior to measuring amplification kinetics at 65° C. Representative amplification curves determined by measuring EvaGreen fluorescence (Y-axis) over incubation time (X-axis; hh:mm:ss).

FIG. 26. Threshold cycle (Cₜ) analysis of triple and quadruple supercharged vHP47 variants. LAMP assay results shown in FIG. 16C were further quantified by Cₜ values. Threshold cycle for amplification of 20 pg (6×10⁷ copies) GAPDH DNA templates was calculated by Lightcycler96 software. Error bar=S.D., n=2 for No Heat, n=3 for 75° C. and 80° C. heat challenge LAMP.

FIG. 27. Protein Thermal Shift Assay for wildtype and mutant Br512 variants. Same amount (40 μg) of parental (Bst-LF) and engineered enzyme variants were analyzed using Protein Thermal Shift™ (Thermo Fisher; Catalog Number: 4461146), a dye-based protein thermal shift assay, according to the manufacturer's instructions. The enzymes were incubated in a Lightcyler 96 (Roche) real-time PCR machine programmed to ramp temperature from 37° C. to 95° C. at the rate of 0.1° C./sec while continuously measuring changes in red fluorescence. Melt curves generated by plotting change in fluorescence (dF) as a function of changing temperature (dT) are depicted. NC: No-protein Control.

FIG. 28. High temperature GAPDH LAMP at 72° C. and 73° C. Identical GAPDH LAMP assays were assembled using equal amounts of either wildtype or indicated mutant Br512 variants and DNA amplification kinetics at 72° C. or 73° C. was determined by measuring changes in EvaGreen fluorescence. Representative amplification curves showing change in fluorescence (Y-axis) over time (X-axis; hh:mm:ss) are depicted.

FIG. 29. Non-template controls in the thermal challenge LAMP assays. (Upper panel) Amplification curves observed in GAPDH LAMP assays containing indicated enzymes and 20 pg (6×10⁷ copies) GAPDH DNA templates are depicted as solid traces while corresponding NTC assays without specific templates are plotted as dotted traces. NTC either present delayed amplification or no amplification. (Y-axis: Fluorescence intensity X-axis: time of incubation). (Lower panels) Melting curve analysis of amplicons generated in LAMP assays with 20 pg (6×10⁷ copies) GAPDH DNA templates (left panel) or NTC LAMP assays without templates (right panel). (Y-axis: −dF/dT: delta Fluorescence/delta temperature X-axis: temperature in ° C.).

FIG. 30. Non-template controls in the high temperature LAMP assays. Representative amplification results from various high temperature GAPDH LAMP assays were plotted. Solid lines indicate amplification curves from reactions seeded with 20 pg (6×10⁷ copies) of GAPDH DNA templates and dotted lines indicate corresponding non-template controls (NTC). Left three panels show amplifications curve (Y-axis: Fluorescence intensity X-axis: time of incubation). Right three panels show melting peaks (Y-axis: −dF/dT: delta Fluorescence/delta temperature X-axis: temperature in ° C.).

FIG. 31. Comparison of supercharged vHP47-Mut Br512 variants, Bst2.0, and Bst3.0 enzymes in 73° C. GAPDH LAMP. GAPDH DNA LAMP assays containing either 16 units of Bst3.0 (NEB) or 16 units of Bst2.0 (NEB) in 1× Isothermal II buffer (NEB) with 8 mM MgSO₄ or containing 20 pmol of wildtype or mutant Br512 variants in the same reaction buffer were incubated at 73° C. and DNA amplification was evaluated by measuring EvaGreen fluorescence. Representative amplification curves showing change in fluorescence (Y-axis) over time (X-axis) are depicted.

FIG. 32. Chaotrope GAPDH DNA LAMP assay using Br512g3 variants. GAPDH DNA LAMP assays containing 20 pmol of Br512 and Br512g3 variants (SC678-Mut235:Br512g3.1, SC5678-Mut235:Br512g3.2) were carried out in the presence of varied amounts (0-2 M) of chaotrope, urea. A total 20 pg (6×10⁷ copies) of GAPDH DNA was seeded to each reaction and DNA LAMP assays were performed at 65° C. DNA amplification was quantified by Evagreen fluorescence. Representative amplification curve was plotted as graphs. (Y-axis: Fluorescence, X-axis: time of incubation).

FIGS. 33A-D. High temperature GAPDH LAMP assay at 72° C., 73° C., and 73° C. GAPDH LAMP assays were assembled using 20 pmole of Bst-LF, wildtype Br512 and Mut235 variant. Amplification kinetics at (FIG. 33A) 65° C., (FIG. 33B) 72° C., (FIG. 33C) 73° C. and (FIG. 33D) 74° C. were determined by measuring EvaGreen fluorescence. Representative amplification curves showing changes in fluorescence (Y-axis) over time (X-axis; hh:mm:ss).

FIGS. 34A-E. Comparison of variants in RT-LAMP-OSD assays. Performance of 20 pm of g2.1 (FIG. 34B; positively supercharged variant), g2.2 (FIG. 34C; machine learning variant), g3.1 (FIG. 34D; positively supercharged+machine learning variant) and g3.1+SC4 (FIG. 34E; g3.1+negatively supercharging mutation) was compared to the activity of unmodified ‘parental’ Br512 (FIG. 34A) by amplifying SARS-CoV-2 N gene armored RNA templates using the NB RT-LAMP-OSD assay. OSD fluorescence values measured during (65° C.) and post (37° C.) amplification are depicted as 5000 template copies/reaction, 500 template copies/reaction, 50 template copies/reaction, and 0 template copies/reaction traces. Results representative of two biological replicates are depicted.

FIGS. 35A-D. Comparison of Br512 negative supercharging mutants in RT-LAMP assays. Performance of 20 pm of SC1 (FIG. 35A), SC2 (FIG. 35B), SC3 (FIG. and SC4 (FIG. 35D) negative supercharging variants was compared by amplifying SARS-CoV-2 N gene armored RNA templates using the NB RT-LAMP-OSD assay. OSD fluorescence values measured during (65° C.) and post (37° C.) amplification are depicted as 5000 template copies/reaction, 500 template copies/reaction, 50 template copies/reaction, and template copies/reaction traces. Results representative of two biological replicates are depicted.

### DETAILED DESCRIPTION

In order to improve the core Bst DNAP, the inventors sought to improve its thermostability, so that isothermal amplification reactions could be carried out at progressively higher temperatures, which would concomitantly lead to improved, non-enzymatic strand separation and to potentially faster polymerase kinetics. Enzyme functional improvements are often preceded by general stabilizing mutations (Tokuriki et al., 2008). Improvement of Bst DNAP's thermostability and functionality was achieved through several different, coordinated engineering methods, including the addition of stabilizing domains; the use of machine learning methods to predict stabilizing mutations; and the introduction of charged amino acids that improve interactions with its polyanionic substrates.

In some embodiments, the current disclosure pertains to the stabilization of recombinant proteins using an N-terminal fusion partner. Fusion domains have previously been used in the construction of thermostable DNA polymerases with improved properties for PCR, as opposed to isothermal amplification. In the case of Phusion DNA polymerase, the addition of the Sso7d gene, a DNA binding protein from Sulfolobus solfataricus, stabilizes the polymerase/DNA complex and enhances the processivity by up to 9 times, allowing longer amplicons in less time with less influence of PCR inhibitors (Wang et al., 2004). In order to both increase the thermostability of the Bst DNAP and to provide it with greater interactions with its polyanionic DNA substrate, an extremely robust protein fusion partner was developed based on the villin headpiece (Bazari et al., 1988). The terminal thirty-five amino acids of the headpiece (HP35) consists of three α-helices that form a highly conserved hydrophobic core (Chiu et al., 2005), exhibits co-translational, ultrafast, and autonomous folding properties that may circumvent kinetic traps during protein folding (McKnight et al., 1996). The ultrafast folding property of the villin headpiece subdomain has made it a model for protein folding dynamics and simulation studies (Lei et al., 2007). In addition, HP35 displays thermostability with a transition midpoint (Tₘ) of 70° C. (McKnight et al., 1996; Lei et al., 2007).

As such, provided herein are polymerases (e.g., Bst DNA polymerase or Taq DNA polymerase), and any chimeras thereof, that are stabilized through fusion with a fast-folding protein stabilizer to the protein. More specifically, the terminal forty-seven amino acids of the C-terminal domain of the villin protein from Gallus gallus (“vHP47”; SEQ ID NO: 2) form the N-terminal fusion partner that impart greater solubility and stability on the recombinant protein (e.g., the aforementioned DNA polymerases). In some cases, the terminal forty-seven amino acids may additionally include an initial methionine (see SEQ ID NO: 3). This fast folding partner is larger than the commonly studied HP36 (SEQ ID NO: 1), which is the “head piece” composed of the terminal thirty-six amino acids of the C-terminal domain of the villin protein from G. gallus. Unlike HP36, the “vHP47” fast-folding protein stabilizer forms a complete third alpha helix, which, without wishing to be bound by any theory, functions to further increase its fusion partner's solubility and thermostability. One example of a stabilized recombinant protein provided herein is the Br512 polymerase. This enzyme provides a one-enzyme solution for LAMP as well as RT-LAMP.

There have been various approaches advanced for engineering DNA polymerases for improved function in a variety of settings (Pinheiro, 2019; Nikoomanzar et al., 2020), but these typically focus on a single method for improvement or a single property of the polymerase. It has been hypothesized that impacting different kinetic properties of a protein can lead to additive improvements in protein function (Weber et al., 1990; Mildvan et al., 1992), a hypothesis that also implicitly underlies the many highly successful examples in which DNA shuffling is used to improve protein function (Powell et al., 2001; Sen et al., 2007). Thermal stability is a global property of a protein, and that amino acid changes throughout a protein's structure can lead to a higher melting temperature and performance at higher temperatures (Flores & Ellington, 2002; Matsumura et al., 1999). Therefore, the function of Bst-LF at increasingly higher temperatures was attempted to be improved through several different, complementary mechanisms. The addition of the ultra-fast folding domain HP47 assisted with formation of protein tertiary structure following translation (so-called ‘assisted folding;’ (Kapust & Waugh, 1999; Fox et al., 2003; Yang et al., 2016)) and improved the solubility of the enzyme for purification (Banach et al., 2020; Kapust & Waugh, 1999). Unexpectedly, HP47 also allowed the polymerase to better interact with DNA via its zwitterionic nature, and thereby improved the ability to carry out LAMP reactions. By further using machine-learning methods and supercharging to further improve the stability and functionality of the enzyme in additive ways, provided herein are enzymes with improved stability and functionality. Mutations introduced at multiple sites around the enzyme and its fusion partner generally proved additive, as has previously been observed for structurally distant mutations in other proteins, including transcription factors (Tongtur et al., 2010), kinesin (Richard et al., 2016), and serine proteases (Oskarsson et al., 2020). Ultimately, enzymes (Br512g3.1 and Br512g3.2) were generated that perform high temperature (74° C.), ultra-fast (6 minute) LAMP reactions, and still provide reliable and consistent outputs. Overall, these combined enzyme engineering efforts generate multiple new options for carrying out and translating LAMP reactions into practice, including in point-of-care settings.

### I. RECOMBINANT PROTEINS

A. N-Terminal Stabilizers

Expression of recombinant proteins in microbial host cells is widely used for industrial production of enzymatic proteins for in vitro use. In some cases, a fusion partner may be attached to the N-terminal end of the target protein. These N-terminal fusion partners (i.e., N-terminal stabilizers) function to increase expression, solubility, stability (e.g., thermostability), and/or correct folding of the target protein. Furthermore, these fusion partners (1) provide molecular handles for further protein augmentation and (2) stabilize chimeras and other rational designs to assist in the development of new protein (e.g., polymerase) variants.

The N-terminal fusion partners comprise the terminal forty-seven amino acids of the C-terminal domain of the villin protein from Gallus gallus (“vHP47”; SEQ ID NO: 2) form the N-terminal fusion partner that impart greater solubility and stability on the recombinant protein without affecting enzyme function. In some cases, the terminal forty-seven amino acids may additionally include an initial methionine (see SEQ ID NO: 3).

In various aspects, the N-terminal fusion partner may comprise a sequence that is at least or about 85%, at least or about 86%, at least or about 87%, at least or about 88%, at least or about 89%, at least or about 90%, at least or about 91%, at least or about 92%, at least or about 93%, at least or about 94%, at least or about 95%, at least or about 96%, at least or about 97%, at least or about 98%, or at least or about 99% identical to the sequence of SEQ ID NO: 2. In some aspects, the N-terminal fusion partner may comprise a sequence that is identical to the sequence of SEQ ID NO: 2.

As used herein, “N-terminal” fusion refers to a fusion partner located N-terminal to a protein of interest, such as an enzyme (e.g., a polymerase). The N-terminal fusion partner, vHP47, can have additional amino acid or proteins further N-terminal relative to its position.

B. Heterologous Protein Domains

In the recombinant proteins of the present disclosure, the heterologous protein domain is the feature ultimately being enhanced by the presence of the N-terminal fusion partner. The heterologous protein domain may be any useful protein domain that may benefit from the increased stability and solubility imparted by the presence of the N-terminal fusion partner. In some aspects, the heterologous protein domain may be biologically active and/or enzymatically active. The heterologous protein domain may be a naturally occurring protein, an engineered protein, a variant of a naturally occurring or engineered protein, or a fragment of a naturally occurring or engineered protein.

In various aspects, the heterologous protein domain is an enzyme selected from the following group of enzymes: polymerase (e.g., DNA polymerase, RNA polymerase, reverse transcriptase), amylase, protease, kinase, phosphatase, integrase, luciferase, cellulase, ligninase, lipase, mannanase, glucanase, amyloglucosidase, pectinase, ligase, nuclease, oxidase, dehydrogenase, reductase, oxidoreductases, methyltransferase, glycosyl hydrolase, lyase, mono or dioxidase, peroxidase, transaminase, carboxypeptidase, amidase, esterase, and isomerase.

Other examples of heterologous protein domains include transforming growth factor α (TGF-α), transforming growth factor β (TGF-β), epidermal growth factor (EGF), vascular endothelial growth factor (VEGF), thrombopoietin (TPO), interferon, pro-urokinase, urokinase, plasminogen activator inhibitor 1, plasminogen activator inhibitor 2, von Willebrandt factor, a cytokine, e.g. an interleukin such as interleukin (IL) 1, IL-I Ra, IL-2, IL-4, IL-5, IL-6, IL-9, IL-11, IL-12, IL-13, IL-15, IL-16, IL-17, IL-18, IL-20, IL-21, IL-22, IL-23, IL-24, IL-25, IL-26, IL-27, IL-28, IL-29, IL-30, IL-31, IL-32, IL-33, a colony stimulating factor (CFS) such as GM-CSF, stem cell factor, a tumor necrosis factor such as TNF-α, lymphotoxin-α, lymphotoxin-β, CD40L, or CD30L, a protease inhibitor e.g. aprotinin, an enzyme such as superoxide dismutase, asparaginase, arginase, arginine deaminase, adenosine deaminase, ribonuclease, catalase, uricase, bilirubin oxidase, trypsin, papain, alkaline phosphatase, β-glucoronidase, purine nucleoside phosphorylase or batroxobin, an opioid, e.g. endorphins, enkephalins or non-natural opioids, a hormone or neuropeptide, e.g. calcitonin, glucagon, gastrins, adrenocorticotropic hormone (ACTH), cholecystokinins, lutenizing hormone, gonadotropin-releasing hormone, chorionic gonadotropin, corticotrophin-releasing factor, vasopressin, oxytocin, antidiuretic hormones, thyroid-stimulating hormone, thyrotropin-releasing hormone, relaxin, prolactin, peptide YY, neuropeptide Y, pancreatic polypeptide, leptin, CART (cocaine and amphetamine regulated transcript), a CART related peptide, perilipin, melanocortins (melanocyte-stimulating hormones) such as MC-4, melanin-concentrating hormones, natriuretic peptides, adrenomedullin, endothelin, secretin, amylin, vasoactive intestinal peptide (VIP), pituitary adenylate cyclase activating polypeptide (PACAP), bombesin, bombesin-like peptides, thymosin, heparin-binding protein, soluble CD4, hypothalmic releasing factor, and melanotonins.

In some aspects, the heterologous protein domain is a polymerase (e.g., DNA polymerase, RNA polymerase, reverse transcriptase). In various aspects, the polymerase is one that can be used in isothermal amplification reactions. In various aspects, the polymerase is a mesothermophilic (functional up to 70° C.) strand-displacing polymerase. In various aspects, the polymerase is one that is suitable for applications requiring thermophilic strand displacement. In various aspects, the heterologous protein domain is a polymerase derived from a thermophilic bacterium. In various aspects, the polymerase lacks 5′ to 3′ exonuclease activity. In various aspects, the polymerase is an exonuclease-deficient Family A polymerase. In some aspects, the heterologous protein domain is a polymerase from the thermophilic bacterium Thermus aquaticus (Taq) (EC 2.7.7.7), i.e., Taq DNA polymerase, preferably modified to lack 5′ to 3′ exonuclease activity (e.g., Klentaq; SEQ ID NO: 5). In some aspects, the heterologous protein domain is a polymerase from the thermophilic bacterium Bacillus stearothermophilus (Bst), i.e., Bst DNA polymerase, preferably modified to lack 5′ to 3′ exonuclease activity. In some aspects, the Bst DNA polymerase consists of the large fragment (Bst LF; SEQ ID NO: 4), which contains 5′ to 3′ polymerase activity, but lacks 5′ to 3′ exonuclease activity. In some aspects, the polymerase may be a polymerase (e.g., V5.9; SEQ ID NO: 6) as described in U.S. Pat. Publn. 2020/0255891, which is incorporated herein by reference in its entirety.

In various aspects, the polymerase may either be a wildtype enzyme or variant or analogue thereof modified by well-known mutagenesis steps. The enzyme may be further modified, such as comprising new functional groups such as phosphate, acetate, amide groups, or methyl groups, for example. The enzymes may be phosphorylated, glycosylated, lapidated, carbonylated, myristoylated, palmitoylated, isoprenylated, farnesylated, alkylated, hydroxylated, carboxylated, ubiquitinated, deamidated, contain unnatural amino acids by altered genetic codes, contain unnatural amino acids incorporated by engineered synthetase/tRNA pairs, and so forth. The skilled artisan recognizes that post-translational modification of the enzymes may be detected by one or more of a variety of techniques, including at least mass spectrometry, Eastern blotting, Western blotting, or a combination thereof, for example.

C. Augmentative Protein Domains

In various embodiments, the recombinant protein further comprises an augmentative protein domain. The augmentative protein domain may be positioned N-terminally relative to the stabilizer domain. The augmentative protein domain may be a DNA binding protein (DBP). For example, the DBP may be a single stranded DBP, such as, an extreme thermostable single-stranded DNA binding protein (ET SSB), E. coli recA, T7 gene 2.5 product, phage lambda RedB or Rac prophage RecT. Many other single stranded DNA binding proteins are known and could be used in the recombinant protein. The augmentative protein domain may be an RNA binding domain (RBD). Non-limiting examples of proteins or peptides that contain RNA binding domains include Puf family of proteins (e.g., pumilio), RRM (e.g., RNA recognition motif) proteins, double-stranded RNA-binding motif (dsRM, dsRBD) proteins, staufen family of proteins, KH type I and type II family of proteins, hnRNP family of proteins, bacteriophage P22 N protein, and bacteriophage MS2 coat protein.

D. Linkers

In various embodiments, the stabilizer domain may be attached directly to the N-terminal amino acid of the recombinant protein via a peptide bond. In other various embodiments, the stabilizer domain may be attached to the recombinant protein via a linker group (e.g., a proteinaceous linker). As such, the linker group may be positioned between the stabilizer domain and the heterologous protein domain such that the heterologous protein domain comprises an N-terminal extension comprising a combination of the stabilizer domain and the linker group. In embodiments where the recombinant protein comprises an augmentative protein domain positioned N-terminally relative to the stabilizer domain, a first linker may be positioned between the augmentative protein domain and the stabilizer domain, and a second linker may be positioned between the stabilizer domain and the heterologous protein domain. In some aspects where two or more linkers are present in a recombinant protein, each linker may be the same or the various linkers may be selected independently.

In various embodiments, the linker has from 1-30, from 1-25, from 1-20, from 1-15, from 1-14, from 1-13, from 1-12, from 1-11, from 1-10, from 1-9, from 1-8, from 1-7, from 1-6, from 1-5 amino, from 1-4, from 1-3, from 2-30, from 2-25, from 2-20, from 2-15, from 2-14, from 2-13, from 2-12, from 2-11, from 2-10, from 2-9, from 2-8, from 2-7, from 2-6, from 2-5, from 2-4, from 3-30, from 3-25, from 3-20, from 3-15, from 3-14, from 3-13, from 3-12, from 3-11, from 3-10, from 3-9, from 3-8, from 3-7, from 3-6, from 3-5, from 3-4, from 4-30, from 4-25, from 4-20, from 4-15, from 4-14, from 4-13, from 4-12, from 4-11, from 4-10, from 4-9, from 4-8, from 4-7, from 4-6, from 4-5, from 5-30, from 5-25, from 5-20, from 5-15, from 5-14, from 5-13, from 5-12, from 5-11, from 5-10, from 5-9, from 5-8, from 5-7, from 5-6, from 6-30, from 6-25, from 6-20, from 6-15, from 6-14, from 6-13, from 6-12, from 6-11, from 6-10, from 6-9, from 6-8, from 6-7, from 7-30, from 7-25, from 7-20, from 7-15, from 7-14, from 7-13, from 7-12, from 7-11, from 7-10, from 7-9, or from 6-8 amino acid residues.

In various embodiments, the linker will comprise amino acid residues that render the linker a flexible structure, such as alternating Ser and Gly residues. Non-limiting examples of linker groups include GSGSAAAP (SEQ ID NO: 8), SSSGSSGSSGSS (SEQ ID NO: 9), GGSSGGSS (SEQ ID NO: 10), SSSGSGSG (SEQ ID NO: 11), ALALALA (SEQ ID NO: 12), ALALALAPA (SEQ ID NO: 13), SSSALALALA (SEQ ID NO: 14), SGSGSGSGS (SEQ ID NO: 15), SSSGSGSGSG (SEQ ID NO: 16), GSSGSGS(SEQ ID NO: 17), and GGGGSGGGGSGGGGS (SEQ ID NO: 18).

In various embodiments, the linker group may be a cleavable peptide. In some cases, the cleavable peptide may be a self-cleavable peptide, such as, for example, a 2A peptide. The 2A peptide may be a T2A peptide, a P2A peptide, an E2A peptide, or a F2A peptide. The presence of this peptide provides for separation of the stabilizer from the homologous protein domain following translation and folding. In some cases, the cleavable peptide may be a cleavage site for a protease.

E. Modifications

Modified recombinant proteins may possess deletions and/or substitutions of amino acids; thus, a recombinant protein with a deletion, a recombinant protein with a substitution, and a recombinant protein with a deletion and a substitution are modified recombinant proteins. These modified recombinant proteins may further include insertions or added amino acids, such as with fusion proteins or proteins with linkers, for example. A “modified deleted recombinant proteins” lacks one or more residues of a recombinant protein, but may possess the specificity and/or activity of the wild-type recombinant protein.

Substitution or replacement variants may contain the exchange of one amino acid for another at one or more sites within the recombinant protein and may be designed to modulate one or more properties of the recombinant polypeptide. Substitutions may or may not be conservative, that is, one amino acid is replaced with one of similar size and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

Amino acid and nucleic acid sequences may include additional residues, such as additional N- or C-terminal amino acids or 5′ or 3′ sequences, and yet still be essentially as set forth in one of the sequences disclosed herein. The addition of terminal sequences particularly applies to nucleic acid sequences that may, for example, include various non-coding sequences flanking either of the 5′ or 3′ portions of the coding region or may include various internal sequences.

### II. NUCLEIC ACIDS AND VECTORS

Nucleic acid sequences encoding a recombinant protein of the present disclosure are also contemplated. Depending on which expression system is used, nucleic acid sequences can be selected based on conventional methods. For example, if the recombinant protein is derived from (or portions of the recombinant protein are derived from) a first species and contains one or more codons that are rarely used in organism used for expression (e.g., E. coli), then that may interfere with expression. Therefore, the nucleic acid sequences may be codon optimized for E. coli expression using freely available software to design coding sequences free of rare codons as to the organism used for expression. Various vectors may be also used to express the recombinant protein. Exemplary vectors include, but are not limited, plasmid vectors, phages, viral vectors, transposons, or liposome-based vectors.

### III. HOST CELLS

Host cells may be any that may be transformed to allow the expression of a recombinant protein of the present disclosure. The host cells may be bacteria, mammalian cells, yeast, or filamentous fungi. Various bacteria include Escherichia and Bacillus. Yeasts belonging to the genera Saccharomyces, Kiuyveromyces, Hansenula, or Pichia would find use as an appropriate host cell. Various species of filamentous fungi may be used as expression hosts, including the following genera: Aspergillus, Trichoderma, Neurospora, Penicillium, Cephalosporium, Achlya, Podospora, Endothia, Mucor, Cochliobolus, and Pyricularia.

Examples of usable host organisms include bacteria, e.g., Escherichia coli MC1061, derivatives of Bacillus subtilis BRB1 (Sibakov et al., 1984), Staphylococcus aureus SA1123 (Lordanescu, 1975) or Streptococcus lividans (Hopwood et al., 1985); yeasts, e.g., Saccharomyces cerevisiae AH 22 (Mellor et al., 1983) or Schizosaccharomyces pombe; and filamentous fungi, e.g., Aspergillus nidulans, Aspergillus awamori (Ward, 1989), or Trichoderma reesei (Penttila et al., 1987; Harkki et al., 1989).

### IV. PROTEIN PURIFICATION

Protein purification techniques are well known to those of skill in the art. These techniques involve, at one level, the homogenization and crude fractionation of the host cells to polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure peptide are ion-exchange chromatography, gel exclusion chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC).

A purified protein or peptide is intended to refer to a composition, isolatable from other components, wherein the protein or peptide is purified to any degree relative to its naturally obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various techniques suitable for use in protein purification are well known to those of skill in the art. These include, for example, precipitation with ammonium sulfate, PEG, antibodies and the like, or by heat denaturation, followed by centrifugation; chromatography steps, such as ion exchange, gel filtration, reverse phase, hydroxyapatite, and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

Various methods for quantifying the degree of purification of the protein or peptide are known to those of skill in the art. These include, for example, determining the specific activity of an active fraction, or assessing the amount of polypeptides within a fraction by sodium dodecyl sulfate polyacrylamide gel electrophoresis (SDS/PAGE) analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

There is no general requirement that the protein or peptide will always be provided in its most purified state. Indeed, it is contemplated that less substantially purified products may have utility. Partial purification may be accomplished by using fewer purification steps in combination, or by utilizing different forms of the same general purification scheme. For example, it is appreciated that a cation-exchange column chromatography performed utilizing a high-performance liquid chromatography (HPLC) apparatus will generally result in a greater “-fold” purification than the same technique utilizing a low-pressure chromatography system. Methods exhibiting a lower degree of relative purification may have advantages in total recovery of protein product, or in maintaining the activity of an expressed protein.

A protein or peptide may be isolated or purified. For example, a His tag or an affinity epitope may be comprised in a recombinant protein to facilitate purification. Affinity chromatography is a chromatographic procedure that relies on the specific affinity between a substance (e.g., a recombinant protein having a His tag) to be isolated and a molecule to which it can specifically bind (e.g., metal ions). This is a receptor-ligand type of interaction. The column material is synthesized by covalently coupling one of the binding partners (e.g., nickel) to an insoluble matrix (e.g., nitrilotriacetic acid (NTA) agarose). The column material is then able to specifically adsorb the substance from the solution. Elution occurs by changing the conditions to those in which binding will not occur (e.g., adding imidazole or altering pH, ionic strength, temperature, etc.). The matrix should be a substance that does not adsorb non-target molecules to any significant extent and that has a broad range of chemical, physical, and thermal stability. The ligand should be coupled in such a way as to not affect its binding properties. The ligand should also provide relatively tight binding. It should be possible to elute the substance without destroying the sample or the ligand.

Size exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase.

The underlying principle of SEC is that particles of different sizes will elute (filter) through a stationary phase at different rates. This results in the separation of a solution of particles based on size. Provided that all the particles are loaded simultaneously or near simultaneously, particles of the same size should elute together. Each size exclusion column has a range of molecular weights that can be separated. The exclusion limit defines the molecular weight at the upper end of this range and is where molecules are too large to be trapped in the stationary phase. The permeation limit defines the molecular weight at the lower end of the range of separation and is where molecules of a small enough size can penetrate into the pores of the stationary phase completely and all molecules below this molecular mass are so small that they elute as a single band.

High-performance liquid chromatography (or high-pressure liquid chromatography, HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used.

### V. METHODS OF USE

Disclosed herein are non-naturally occurring stabilized polymerases, wherein thermostable polymerases are characterized by increased temperature stability in the range of 70° C. to 100° C., increased strand displacement capability, increased processivity, or a combination thereof compared with a wild type large fragment Bacillus stearothermophilus (Bst LF) polymerase. The polymerase is also more thermostable than Bst 2.0. The stabilized polymerases are capable of both isothermal amplification, such as LAMP and hyperbranched rolling circle amplification (hbRCA) from DNA templates as well as from RNA templates without the need to include a separate reverse transcriptase. In some aspects, the recombinant enzymes of the disclosure are used for both reverse transcription and subsequent amplification of cDNA. They are also useful with strand displacement amplification (SDA), polymerase spiral reaction (PSR), or helicase dependent amplification (HDA). The polymerases are also capable of replicating DNA in a polymerase chain reaction (PCR). In certain embodiments, the recombinant enzymes of the disclosure are used for molecular biology applications, such as diagnostics (such as analyzing nucleic acids from a biological sample or derived from nucleic acids from a biological sample), cDNA library cloning, and next-generation RNA sequencing.

The methods and products disclosed herein can be used for multiple applications. Detection and identification of virtually any nucleic acid sequence can be accomplished. For example, the presence of specific viruses, microorganisms and parasites can be detected. For example, SARS-CoV-2 genomic material can be amplified. Genetic diseases can also be detected and diagnosed, either by detection of sequence variations (mutations) which cause or are associated with a disease or are linked (Restriction Fragment Length Polymorphisms or RFLPs) to the disease locus. Sequence variations which are associated with, or cause, cancer, can also be detected. This can allow for both the diagnosis and prognosis of disease. For example, if a breast cancer marker is detected in an individual, the individual can be made aware of their increased likelihood of developing breast cancer, and can be treated accordingly. The methods and devices disclosed herein can also be used in the detection and identification of nucleic acid sequences for forensic fingerprinting, tissue typing and for taxonomic purposes, namely the identification and speciation of microorganisms, flora and fauna.

The methods and devices disclosed herein have applications in clinical medicine, veterinary science, aquaculture, horticulture and agriculture. The methods and devices can also be used in maternity and paternity testing, fetal sex determination, and pregnancy tests.

For example, isothermal amplification methods seek to amplify DNA or RNA via continuous replication at a single temperature, which enables the creation of a variety of fascinating and useful point of care devices. Furthermore, rolling circle amplification (RCA) and loop-mediated isothermal amplification (LAMP; U.S. Pat. Publn. 2016/0076083, which is incorporated herein by reference in its entirety) require only polymerases and primers. RCA can proceed at mesophilic or higher temperatures, amplifying continuously around a circular template to generate long, concatenated DNA products. When initiated from a nick or single primer, amplification is linear; by including both forward and reverse primers, however, amplification becomes exponential, generating 10⁹-fold amplification in 90 minutes from 10⁸ copies of template in a reaction commonly referred to as hyperbranched RCA (hbRCA). LAMP, also exponential, is currently an inherently higher temperature mechanism, using 4-6 primers to generate 10⁹-fold amplification of short (100-500 bp) DNA targets in an hour or less by creating ladder-like concatenated amplicons. Both methods are rapid, single-enzyme nucleic acid detection systems that are comparable to PCR in terms of sensitivity, yet are faster and can operate isothermally.

LAMP can be conducted with two, three, four, five, or six primers, for example. OSD-LAMP can be used with 2 primers (FIP+BIP) and also 3 primers (FIP+BIP+F3 and FIP+BIP+B3). 2 as well as 3-primer OSD-LAMP assays can also be used. The 4-primer LAMP is the basic form of LAMP that was originally described for isothermal nucleic acid amplification. The system is composed of two loop-forming inner primers FIP and BIP and two outer primers F3 and B3 whose primary function is to displace the DNA strands initiated from the inner primers thus allowing formation of the loops and strand displacement DNA synthesis. Subsequently, 6-primer LAMP was reported that incorporated 2 additional primers, LF and LB, that bind to the loop sequences located between the F1/F1c and F2/F2c priming sites and the B1/B1c and B2/B2c priming sites. Addition of both loop primers significantly accelerated LAMP. Stem primers that go between F1 and B1 regions have also been described. The 5-primer LAMP has been described, wherein the 4 LAMP primers (F3, B3, FIP and BIP) are used in conjunction with only one of the loop primers (either LF or LB). This allows the accelerated amplification afforded by the loop primer while using the other LAMP loop (not bound by the loop primer) for hybridization to loop-specific OSD probe. This allows for high-speed LAMP operation while performing real-time sequence-specific signal transduction.

The isothermal amplification reaction can take place at 20, 25, 30, 35, 40, 45, 50, 55, 60, 65, 70, 75, 80, 85, 90, or 95° C., or any value derivable therein. In one specific embodiment, the isothermal reaction can take place around 40° C. In another specific embodiment, the isothermal reaction can take place around 65° C. In another specific embodiment, the isothermal reaction can take place around 73 or 74° C.

The buffer or the reaction can comprise various components which have been optimized for LAMP. For example, urea can be present in the buffer or the reaction at a concentration of 1.3-1.6 M, or any value derivable therein, for example 1.44 M. The buffer or the reaction can also comprise a stabilized polymerase of the present disclosure at a concentration of 10, 20, 30, 40, 50, 70, 90, 100, 125, 150, 175, 200, 250, 300, 350, 400, 450, 500, 550, 600, 650, 700, 750, 800, 850, 900, 950, 1000, 1250, 1500, 1750, 2000, 2250, 2500, 2750, 3000, 3250, 3500, 3750, 4000, 4250, 4500, 4750, 5000, 5250, 5500, 5750, 6000, 6250, 6500, 6750, 7000, 7250, 7500, 7750, 8000, 8250, 8500, 8750, 9000, 9250, 9500, 9750, 10,000, or more nM, or any value derivable therein. The buffer or the reaction can comprise MgSO₄ and/or MgCl₂, MgSO₄ and/or MgCl₂ can be present at a concentration of 1.0, 1.1. 1.2, 1.3, 1.4, 1.5, 1.6, 1.7, 1.8, 1.9, 2.0, 2.1, 2.2, 2.3, 2.4, or 2.5 or more mM, or any value derivable therein. The buffer or the reaction can also comprise a Single-Stranded Binding (SSB) protein. SSB can be present at a concentration of about 0.2 to 0.7 μg, or any value derivable therein, for example about 0.5 μg.

In the method disclosed herein, the strand displacement reporter can be one step toehold displacement (OSD) reporter. The target nucleic acid can be RNA or DNA. Four, five, or six primers can be used with the isothermal amplification reaction.

Amplification of the target nucleic acid takes place in real time. Many examples of real-time amplification are known to those of skill in the art. One of skill in the art could therefore readily ascertain a real-time method for use with the invention disclosed herein.

Also disclosed is a method of diagnosing a subject with a disease, the method comprising carrying out the method of amplification described herein, wherein the presence of a target nucleic acid indicates the presence of a disease in the subject.

LAMP can be carried out using DNA or RNA (RT-LAMP). RT-LAMP, when performed using a stabilized polymerase of the present disclosure, may not need to include a separate reverse transcription step and thus may be not need to use an exogenous reverse transcriptase. LAMP can amplify nucleic acids from a wide variety of samples. These include, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); plant materials; biological warfare agent samples; research samples (for example, the sample may be the product of an amplification reaction, for example general amplification of genomic DNA); purified samples, such as purified genomic DNA, RNA, proteins, etc.; raw samples (bacteria, virus, genomic DNA, etc.); as will be appreciated by those in the art, virtually any experimental manipulation may have been done on the sample. Specifically, it is noted that the polymerases disclosed herein can be used to amplify SARS-CoV-2. Some embodiments utilize siRNA and microRNA as target sequences.

Some embodiments utilize nucleic acid samples from stored (e.g. frozen and/or archived) or fresh tissues. Paraffin-embedded samples are of particular use in many embodiments, as these samples can be very useful, due to the presence of additional data associated with the samples, such as diagnosis and prognosis. Fixed and paraffin-embedded tissue samples as described herein refers to storable or archival tissue samples. Most patient-derived pathological samples are routinely fixed and paraffin-embedded to allow for histological analysis and subsequent archival storage.

### VI. KITS

The invention is also drawn to compositions and kits which contain a recombinant protein of the disclosure, as well as the use of the recombinant protein in any methodology where such proteins are employed. A “kit” refers to a combination of physical elements. For example, a kit may comprise one or more stabilized polymerase (optionally lyophilized) as described herein and optionally instructions for their use. Kits may also comprise one or more reaction buffer, oligonucleotide primer, NTP or dNTP mix, and other elements useful in the use of a recombinant protein described herein.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present invention also will typically include a means for containing the recombinant protein, and any other reagent containers in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the invention. Such kits, however, are not limited to the particular items identified above and may include any reagent useful in the use of the recombinant protein.

### VII. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the terms “enzyme” and “protein” and “polypeptide” refer to compounds comprising amino acids joined via peptide bonds and are used interchangeably.

As used herein, the term “fusion protein” refers to a chimeric protein containing proteins or protein fragments operably linked in a non-native way.

The terms “in operable combination,” “in operable order,” and “operably linked” refer to a linkage wherein the components so described are in a relationship permitting them to function in their intended manner, for example, a linkage of nucleic acid sequences in such a manner that a nucleic acid molecule capable of directing the transcription of a given gene and/or the synthesis of desired protein molecule, or a linkage of amino acid sequences in such a manner so that a fusion protein is produced.

The term “gene” refers to a DNA sequence that comprises control and coding sequences necessary for the production of a polypeptide or precursor thereof. The polypeptide can be encoded by a full-length coding sequence or by any portion of the coding sequence so as the desired enzymatic activity is retained.

The term “sample” is used herein in its broadest sense and can be, by non-limiting example, any sample that is suspected of containing a target agent(s) to be detected. It is meant to include specimens or cultures (e.g., microbiological cultures), and biological and environmental specimens as well as non-biological specimens. Biological samples may comprise animal-derived materials, including fluid (e.g., blood, saliva, urine, lymph, etc.), solid (e.g., stool) or tissue (e.g., buccal, organ-specific, skin, etc.), as well as liquid and solid food and feed products and ingredients such as dairy items, vegetables, meat and meat by-products, and waste. Biological samples may be obtained from, e.g., humans, any domestic or wild animals, plants, bacteria or other microorganisms, etc. Environmental samples can include environmental material such as surface matter, soil, water (e.g., contaminated water), air and industrial samples, as well as samples obtained from food and dairy processing instruments, apparatus, equipment, utensils, disposable and non-disposable items. These examples are not to be construed as limiting the sample types applicable to the present invention. Those of skill in the art would appreciate and understand the particular type of sample required for the detection of particular target agents.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide” are used interchangeably herein. Accordingly, the term “protein or peptide” encompasses amino acid sequences comprising at least two of the 20 common amino acids found in naturally occurring proteins, or at least one modified or non-natural amino acid.

Proteins or peptides may be made by any technique known to those of skill in the art, including the expression of proteins, polypeptides, or peptides through standard molecular biological techniques, the isolation of proteins or peptides from natural sources, or the chemical synthesis of proteins or peptides. The coding regions for known genes may be amplified and/or expressed using the techniques disclosed herein or as would be known to those of ordinary skill in the art. Alternatively, various commercial preparations of proteins, polypeptides, and peptides are known to those of skill in the art.

The term “native” refers to the typical or wild-type form of a gene, a gene product, or a characteristic of that gene or gene product when isolated from a naturally occurring source. In contrast, the term “modified,” “variant,” “mutein,” or “mutant” refers to a gene or gene product that displays modification in sequence and functional properties (i.e., altered characteristics) when compared to the native gene or gene product, wherein the modified gene or gene product is genetically engineered and not naturally present or occurring.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for an RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

Chemicals and reagents. All chemicals were of analytical grade and were purchased from Sigma-Aldrich (St. Louis, MO, USA) unless otherwise indicated. All commercially sourced enzymes and related buffers were purchased from New England Biolabs (NEB, Ipswich, MA, USA) unless otherwise indicated. All oligonucleotides and gene blocks (Table 1) were obtained from Integrated DNA Technologies (IDT, Coralville, IA, USA). SARS-CoV-2 genomic RNA and inactivated virions were obtained from American Type Culture Collection, Manassas, VA, USA.

TABLE 1
Oligonucleotide and template sequences used in the study.

| Name | Sequence | Use |
|---|---|---|
| gapdLAMP.F3 | GCCACCCAGAAGACTGTG (SEQ ID NO: 22) | gapd |
| gapdLAMP.B3 | TGGCAGGTTTTTCTAGACGG (SEQ ID NO: 23) | LAMP- |
| gapdLAMP.FIP | CGCCAGTAGAGGCAGGGATGAGGGAAACTGTGGCGTGAT | OSD |
| | (SEQ ID NO: 24) | |
| gapdLAMP.BIP | GGTCATCCCTGAGCTGAACGGTCAGGTCCACCACTGACAC | |
| | (SEQ ID NO: 25) | |
| gapdLAMP.LR | TGTTCTGGAGAGCCCCGCGGCC (SEQ ID NO: 26) | |
| gapdOSD.F | /56- | |
| | FAM/CTCACTGGCATGGCCTTCCGTGTCCCCACTGCCAAC | |
| | /3InvdT/ (SEQ ID NO: 27) | |
| gapdOSD.Q | GGACACGGAAGGCCATGCCAGTGAG/3IABKFQ/ (SEQ | |
| | ID NO: 28) | |
| gapd template | CTAGTAACGGCCGCCAGTGTGCTGGAATTCCCACAGTCCA | |
| | TGCCATCACTGCCACCCAGAAGACTGTGGATGGCCCCTCC | |
| | GGGAAACTGTGGCGTGATGGCCGCGGGGCTCTCCAGAACA | |
| | TCATCCCTGCCTCTACTGGCGCTGCCAAGGCTGTGGGCAA | |
| | GGTCATCCCTGAGCTGAACGGGAAGCTCACTGGCATGGCC | |
| | TTCCGTGTCCCCACTGCCAACGTGTCAGTGGTGGACCTGA | |
| | CCTGCCGTCTAGAAAAACCTGCCAAATATGATGACATCAA | |
| | GAAGGTGGTGAAGCAGGCGTCGGAGGGCCCCCTCAAGGGC | |
| | ATCCTGGGCTACACTGAGCACCAGGTGGTCTCCTCTGACT | |
| | TCAACAGCGACACCCACTCCTCCACCTTTGACGCTGGGGC | |
| | TGGCATTGCCCTCAACGACCACTTTGTCAAGCTCATTTCC | |
| | TGGAATTCTGCAGATATCCATCACACTGGCGGCCGCTCGA | |
| | GC (SEQ ID NO: 29) | |
| NB-F3 | ACCGAAGAGCTACCAGACG (SEQ ID NO: 30) | NB RT- |
| NB-B3 | TGCAGCATTGTTAGCAGGAT (SEQ ID NO: 31) | LAMP- |
| NB-FIP | TCTGGCCCAGTTCCTAGGTAGTTCGTGGTGGTGACGGTAA | OSD |
| | (SEQ ID NO: 32) | |
| NB-BIP | AGACGGCATCATATGGGTTGCACGGGTGCCAATGTGATCT | |
| | (SEQ ID NO: 33) | |
| NB-LB | ACTGAGGGAGCCTTGAATACA (SEQ ID NO: 34) | |
| NB-OSD-FAM | /56- | |
| | FAM/CCGAATGAAAGATCTCAGTCCAAGATGGTATTTCT/ | |
| | 3InvdT/ (SEQ ID NO: 35) | |
| NB-OSD-Q | TCTTGGACTGAGATCTTTCATTCGG/3IABKFQ/ (SEQ | |
| | ID NO: 36) | |
| Lamb-F3 | TCCAGATGAGGATGAAGAAGA (SEQ ID NO: 37) | 6-Lamb |
| Lamb-B3 | AGTCTGAACAACTGGTGTAAG (SEQ ID NO: 38) | RT-LAMP- |
| Lamb-FIP | AGAGCAGCAGAAGTGGCACAGGTGATTGTGAAGAAGAAGA | OSD |
| | G (SEQ ID NO: 39) | |
| Lamb-BIP | TCAACCTGAAGAAGAGCAAGAACTGATTGTCCTCACTGCC | |
| | (SEQ ID NO: 40) | |
| Lamb-LF | CTCATATTGAGTTGATGGCTCA (SEQ ID NO: 41) | |
| Lamb-LB | ACAAACTGTTGGTCAACAAGAC (SEQ ID NO: 42) | |
| Lamb-OSD- | GTATGGTACTGAAGATGATTACCAAGGTAAACCTTTGGAA | |
| FAM | TTTGGAC/36-FAM/ (SEQ ID NO: 43) | |
| Lamb-OSD-Q | /5IABKFQ/GTCCAAATTCCAAAGGTTTACCTTGGTAATC | |
| | ATCTC/3InvdT/ (SEQ ID NO: 44) | |
| Tholoth-F3 | TGCTTCAGTCAGCTGATG (SEQ ID NO: 45) | Tholoth |
| Tholoth-B3 | TTAAATTGTCATCTTCGTCCTT (SEQ ID NO: 46) | RT-LAMP- |
| Tholoth-FIP | TCAGTACTAGTGCCTGTGCCCACAATCGTTTTTAAACGGG | OSD |
| | T (SEQ ID NO: 47) | |
| Tholoth-BIP | TCGTATACAGGGCTTTTGACATCTATCTTGGAAGCGACAA | |
| | CAA (SEQ ID NO: 48) | |
| Tholoth-LB | GTAGCTGGTTTTGCTAAATTCC (SEQ ID NO: 49) | |
| Tholoth-OSD- | /56- | |
| FAM | FAM/ACAGGTGTAAGTGCAGCCCGTCTTACACCGTGC/ | |
| | 3InvdT/ (SEQ ID NO: 50) | |
| Tholoth-OSD-Q | GACGGGCTGCACTTACACCTGT/3IABKFQ/ (SEQ ID | |
| | NO: 51) | |

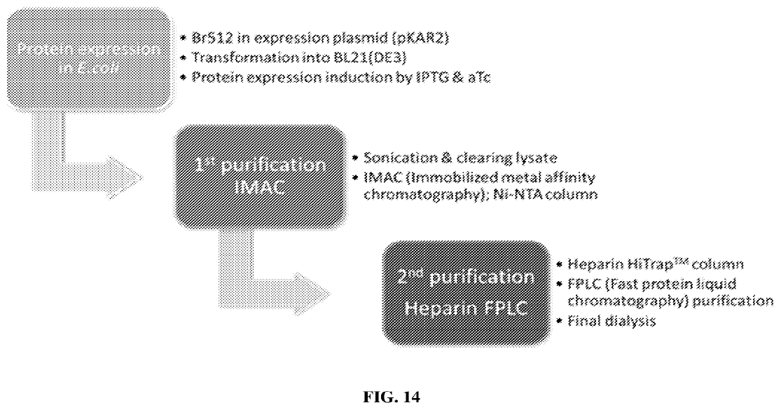

Br512 purification protocol. The overall scheme for Br512 purification is shown in FIG. 14. In short, Br512 (amino acid sequence of SEQ ID NO: 19; encoded by nucleotide sequence of SEQ ID NO: 20) was cloned into an in-house E. coli expression vector under the control of a T7 RNA polymerase promoter (pKAR2). The Br512 expression construct (pKAR2-Br512) and its variants were then transformed into E. coli BL21(DE3) (NEB, C2527H). A single colony was seed cultured overnight in 5 mL of superior broth (Athena Enzyme Systems, 0105). The next day, 1 mL of seed culture was inoculated into 1 L of superior broth and grown at 37° C. until it reached an OD600 of 0.5-0.6 or 0.7-0.8. Enzyme expression was induced with 1 mM IPTG and 100 ng/mL of anhydrous tetracycline (aTc) at 18° C. for 18 h (or overnight). The induced cells were pelleted at 5000×g for 10 min at 4° C. and resuspended in 30 mL of ice-cold lysis buffer (50 mM Phosphate Buffer, pH 7.5, 300 mM NaCl, 20 mM imidazole, 0.1% Igepal CO-630, 5 mM MgSO₄, 1 mg/mL HEW Lysozyme, 1×EDTA-free protease inhibitor tablet, Thermo Scientific, A32965). The samples were then sonicated (1 sec ON, 4 sec OFF) for a total of 4 minutes with 40% amplitude. The lysate was centrifuged at 35,000×g for 30 min at 4° C. The supernatant was transferred to a clean tube and filtered through a 0.2 μm filter.

Protein from the supernatant was purified using metal affinity chromatography on an Ni-NTA column. Briefly, 1 mL of Ni-NTA agarose slurry was packed into a 10 mL disposable column and equilibrated with 20 column volume (CV) of equilibration buffer (50 mM Phosphate Buffer, pH 7.5, 300 mM NaCl, 20 mM imidazole). The sample lysate was loaded onto the column and the column was developed by gravity flow. Following loading, the column was washed with 20 CV of equilibration buffer and 5 CV of wash buffer (50 mM Phosphate Buffer, pH 7.5, 300 mM NaCl, 50 mM imidazole). Br512 was eluted with 5 mL of elution buffer (50 mM Phosphate Buffer, pH 7.5, 300 mM NaCl, 250 mM imidazole). The eluate was dialyzed twice with 2 L of Ni-NTA dialysis buffer (40 mM Tris-HCl, pH 7.5, 100 mM NaCl, 1 mM DTT, 0.1% Igepal CO-630). The dialyzed eluate was further passed through an equilibrated 5 mL heparin column (HiTrap™ Heparin HP) on a FPLC (AKTA pure, GE healthcare) and eluted using a linear NaCl gradient generated from heparin buffers A and B (40 mM Tris-HCl, pH 7.5, 100 mM NaCl for buffer A; 2M NaCl for buffer B, 0.1% Igepal CO-630). The collected final eluate was dialyzed first with 2 L of heparin dialysis buffer (50 mM Tris-HCl, pH 8.0, 50 mM KCl, 0.1% Tween-20) and second with 2 L of final dialysis buffer (50% Glycerol, 50 mM Tris-HCl, pH 8.0, 50 mM KCl, 0.1% Tween-20, 0.1% Igepal CO-630, 1 mM DTT). The purified Br512 was quantified by Bradford assay and SDS-PAGE/coomassie gel staining alongside a bovine serum albumin (BSA) standard.

Site directed mutagenesis. Site directed mutagenesis was performed using Q5® Site-Directed Mutagenesis Kit from NEB (E0554S) according to the manufacturer's instructions. The pKAR2-Br512 plasmid was used as a template to introduce mutations suggested by the Mutcompute analysis. The introduced mutations on the plasmids were confirmed by Sanger sequencing.

Real-time gapd LAMP-OSD. LAMP-OSD reaction mixtures were prepared in 25 μL volume containing indicated amounts of human glyceraldehyde-3-phosphate dehydrogenase (gapd) DNA templates along with a final concentration of 1.6 μM each of BIP and FIP primers, 0.4 μM each of B3 and F3 primers, and 0.8 μM of the loop primer. Amplification was performed in one of the following buffers—1× Isothermal buffer (NEB) (20 mM Tris-HCl, 10 mM (NH4)₂SO₄, 10 mM KCl, 2 mM MgSO₄, 0.1% Triton X-100, pH 8.8), G6B buffer (60 mM Tris-HCl, pH 8.0, 2 mM (NH₄)₂SO₄, 40 mM KCl, 4 mM MgCl₂), G1B buffer (60 mM Tris-HCl, pH 8.0, 5 mM (NH₄)₂SO4, 10 mM KCl, 4 mM MgSO₄, 0.01% Triton X-100), G2A buffer (20 mM Tris-HCl, pH 8.0, 5 mM (NH₄)₂SO₄, 10 mM KCl, 4 mM MgCl₂, 0.01% Triton X-100), or G3A buffer (60 mM Tris-HCl, pH 8.0, 10 mM KCl, 4 mM MgCl₂, 0.01% Triton X-100). The buffer was appended with 1 M betaine, 0.4 mM dNTPs, 2 mM additional MgSO₄ (only for reactions in Isothermal buffer), and either Bst 2.0 DNA polymerase (16 units), Bst-LF DNA polymerase (20 pm), or Br512 DNA polymerase (0.2 pm, 2 μm, 20 μm, or 200 pm). Assays read using OSD probes received 100 nM of OSD reporter prepared by annealing 100 nM fluorophore-labeled OSD strands with a 5-fold excess of the quencher-labeled OSD strands by incubation at 95° C. for 1 min followed by cooling at the rate of 0.1° C./sec to 25° C. Assays read using intercalating dyes received 1× EvaGreen (Biotium, Freemont, CA, USA) instead of OSD probes. For real-time signal measurement, these LAMP reactions were transferred into a 96-well PCR plate, which was incubated in a LightCycler 96 real-time PCR machine (Roche, Basel, Switzerland) maintained at 65° C. for 90 mM. Fluorescence signals were recorded every 3 min in the FAM channel and analyzed using the LightCycler 96 software. For assays read using EvaGreen, amplification was followed by a melt curve analysis on the LightCycler 96 to distinguish target amplicons from spurious background.

Heat challenge and high temperature LAMP. LAMP reaction mixtures were prepared in 25 μL volume containing 10 pg of human glyceraldehyde-3-phosphate dehydrogenase (GAPDH) DNA template plasmid along with a final concentration of 1.6 μM each of BIP and FIP primers, 0.4 μM each of B3 and F3 primers, and 0.8 μM of the loop primer. The reaction mixtures were preassembled on ice and aliquoted into PCR tubes. A total 20 pm of enzyme variants were added to the wells. Amplification was performed in the following buffer (1×LAMP heat challenge buffer: 40 mM Tris-HCl, pH 8.0, 10 mM (NH₄)₂SO₄, 80 mM KCl, 4 mM MgCl₂) supplemented with 0.4 mM dNTP, 1× Evagreen Dye, and 0.4M betaine unless otherwise indicated. For heat challenges, PCR tubes that contain the reaction mixtures and 20 pmol of Br512 enzyme variants were challenged on a PCR machine that was pre-heated to the temperatures indicated in the figures. After the heat challenges, the tubes were immediately removed from the PCR machine and cooled on an ice-cooled metal rack for at least 5 mins. LAMP assay was performed at 65° C. for two hours unless otherwise indicated. Fluorescence signals were recorded every 4 mM in the FAM channel provided by LightCycler 96 software preset.

Endpoint gapd LAMP-OSD. LAMP-OSD reaction mixtures for visual endpoint readout of OSD fluorescence were prepared in 25 μL volume containing either 1× Isothermal buffer and 16 units of Bst 2.0 or G6B buffer and 20 pm of Br512. The reactions also contained 1 M betaine, 1.4 mM dNTPs, 2 mM additional MgSO₄ (only for reactions in Isothermal buffer), and 100 nM OSD reporter prepared by annealing 100 nM fluorophore-labeled OSD strands with a 2-fold excess of the quencher-labeled OSD strands by incubation at 95° C. for 1 mM followed by cooling at the rate of 0.1° C./sec to 25° C. A final concentration of 1.6 μM each of BIP and FIP primers, 0.4 μM each of B3 and F3 primers, and 0.8 μM of the loop primer were added to some reactions while control assays without primers received the same volume of water. Some assays were seeded with 3 μL of human saliva that had been heated at 95° C. for 10 mM while other assays received the same volume of water. All assays were incubated in a thermocycler maintained at 65° C. for 60 mM following which endpoint OSD fluorescence was imaged using a ChemiDoc camera (Bio-Rad Laboratories, Hercules, CA, USA).

SARS-CoV-2 RT-LAMP-OSD assays. Individual 25 lit RT-LAMP-OSD assays were assembled either in 1× Isothermal buffer containing 16 units of Bst 2.0 or in G6D buffer (60 mM Tris-HCl, pH 8.0, 2 mm (NH4)₂SO₄, 40 mM KCl, 8 mM MgCl₂) containing 20 pm of Br512, 20 pm of Bst-LF, or 16 units of Bst 2.0. The buffer was supplemented with 1.4 mM dNTPs, 0.4 M betaine, 6 mM additional MgSO₄ (only for reactions in Isothermal buffer), and 2.4 μM each of FIP and BIP, 1.2 μM of indicated loop primers, and 0.6 μM each of F3 and B3 primers. Amplicon accumulation was measured by adding OSD probes. First, Tholoth, Lamb, and NB OSD probes were prepared by annealing 1 μM of the fluorophore-labeled OSD strand with 2 μM, 3 μM, and 5 μM, respectively of the quencher-labeled strand in 1× Isothermal buffer. Annealing was performed by denaturing the oligonucleotide mix at 95° C. for 1 mM followed by slow cooling at the rate of 0.1° C./s to 25° C. Excess annealed probes were stored at −20° C. Annealed Tholoth, Lamb, and NB OSD probes were added to their respective RT-LAMP reactions at a final concentration of 100 nM of the fluorophore-bearing strand. The assays were seeded with indicated amounts of SARS-CoV-2 viral genomic RNA in TE buffer (10 mM Tris-HCl, pH 7.5, 0.1 mM EDTA) and either incubated for 1 h in a thermocycler maintained at 65° C. for endpoint readout or transferred to a 96-well plate and incubated in the LightCycler 96 real-time PCR machine maintained at 65° C. for real time measurement of amplification kinetics. Endpoint OSD fluorescence was read visually using a blue LED torch with orange filter and imaged using a cellphone camera or a ChemiDoc (Bio-Rad) camera. OSD fluorescence in assays incubated in the real-time PCR machine was measured every 3 mM in the FAM channel and analyzed using the LightCycler 96 software.

Multiplex RT-LAMP-OSD assays comprising 6-Lamb and NB primers and OSD probes were set up using the same conditions as above except that the total LAMP primer amounts were made up of equimolar amounts of 6-Lamb and NB primers supplemented with 0.2 μM each of additional NB FIP and BIP primers. Some multiplex assays received 30 pm Br512 instead of 20 pm enzyme. Some Br512 multiplex assays also received 20 units of the RNase inhibitor, SUPERase. In (Thermo Fisher Scientific, Waltham, MA). Multiplex assays were seeded with indicated amounts of either SARS-CoV-2 viral genomic RNA or inactivated virions in the presence of either 3 μL of TE buffer or human saliva pre-heated at 95° C. for 10 mM. The assays were either incubated for 1 h in a thermocycler maintained at 65° C. for endpoint readout or transferred to a 96-well plate and incubated in the LightCycler 96 real-time PCR machine maintained at 65° C. for real time measurement of amplification kinetics. Endpoint OSD fluorescence was read visually using a blue LED torch with orange filter and imaged using a cellphone camera or a ChemiDoc (Bio-Rad) camera. OSD fluorescence in assays incubated in the real-time PCR machine was measured every 3 mM in the FAM channel and analyzed using the LightCycler 96 software.

Dye-based protein thermal shift assay. The Tm (Transition midpoint; Melting Temperature) of the various enzyme variants were measured using Protein Thermal Shift™ reagents (Thermo Fisher; Catalog Number: 4461146) according to the manufacturer's instruction. Briefly, a total of 40 μg (5 μg/μL) of each enzyme variant in the final dialysis buffer (see Br512 purification protocol) were added into a reaction mixture (20 μL) containing 1× Protein Thermal Shift™ Buffer and 1× Protein Thermal Shift™ Dye. Fluorescence signals were measured in Texas Red channel provided by LightCycler 96 software preset. The red fluorescence change was measured from 37° C. to 95° C. with 0.1° C./sec ramp speed. The measured values (delta Fluorescence/delta Temperature) were plotted on the graph with a Tm calling tool provided by LightCycler 96 analytical software (Roche).

Lyophilization of Br512. Multiplex SARS-CoV-2 assay reagent mixes were prepared by combining dNTPs, NB and 6-Lamb primers and OSD probes, trehalose, and glycerol-free Br512 in the following amounts per individual reaction—(i) 35 nanomoles of dNTPs, (ii) 30 pm each of 6-Lamb FIP and BIP, 15 pm each of 6-Lamb LF and LB loop primers, 7.5 pm each of 6-Lamb F3 and B3 primers, (iii) 35 pm each of NB FIP and BIP, 15 pm of NB LB loop primer, 7.5 pm each of NB F3 and B3 primers, (iv) 2.5 pm of NB fluorophore labeled OSD strand pre-annealed with 5-fold excess of the quencher labeled OSD strand, (v) 2.5 pm of Lamb fluorophore labeled OSD strand pre-annealed with a 3-fold excess of the quencher labeled OSD strand, (vi) 1.25 micromoles of trehalose, and (vii) 20 pm or 30 pm of Br512. The reagent mixes were distributed in 0.2 mL PCR tubes and frozen for 1 h on dry ice prior to lyophilization for 2.5 h at 197 mTorr and −108° C. using the automated settings in a VirTis Benchtop Pro lyophilizer (SP Scientific, Warminster, PA, USA). Lyophilized assays were stored with desiccant at −20° C. until use.

Lyophilized assays were rehydrated immediately prior to use by adding 22 μL of 1×G6D buffer containing 10 micromoles of betaine. Rehydrated assays were seeded with indicated amounts of SARS-CoV-2 viral genomic RNA in a total volume of 3 μL and incubated for 1 h in a thermocycler maintained at 65° C. Endpoint OSD fluorescence was read visually using a blue LED torch with orange filter and imaged using a cellphone camera.

### Example 2—Engineering Fusion Variants of Bst DNAP

The design was centered around the large fragment of DNA polymerase I (Pol I) from G. stearothermophilus (bst, GenBank L42111.1), which is frequently used for isothermal amplification reactions (Tanner & Evans, 2014; Notomi et al., 2000; Hsieh et al., 2014) This fragment (hereafter Bst-LF) lacks a 310 amino acid N-terminal domain that is responsible 5′ to 3′ exonuclease activity, leading to an increased efficiency of dNTP polymerization (Lawyer et al., 1993).

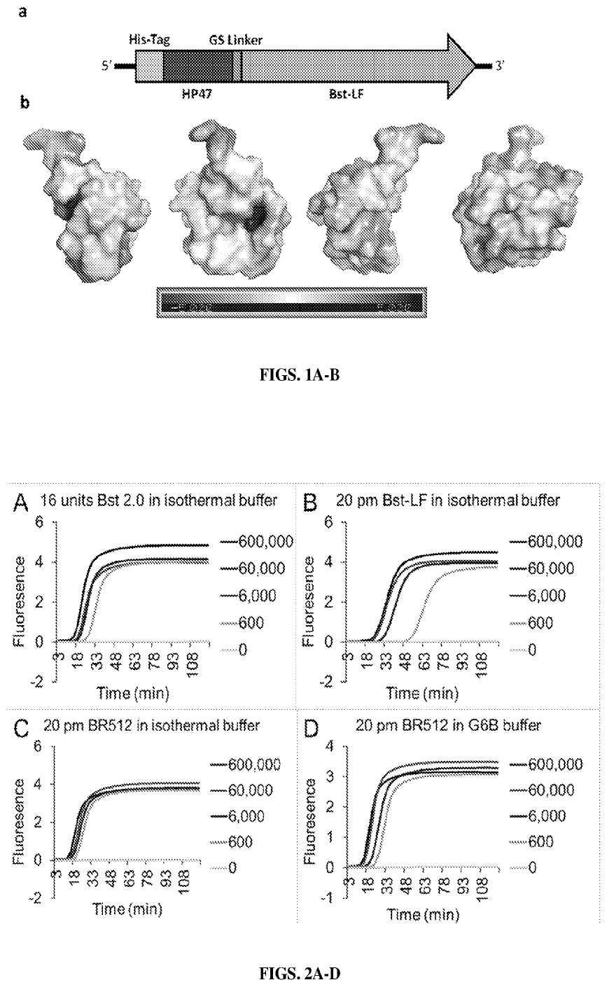

Many larger proteins suffer from slow or inefficient folding (Naganathan & Munoz, 2005), and removal of the N-terminus may lead to increased protein degradation rates, decreased folding speed, and instability (Krishna & Englander, 2005). Indeed, Bst-LF showed low yields upon purification. Therefore, in place of the exonuclease domain, the inventors sought to stabilize Bst DNAP via a fusion partner, the small F-actin binding protein villin, also referred to as the villin headpiece (Bazari et al., 1988). HP35 was extended by twelve amino acids to generate (HP47), where the additional amino acids serve to complete an alpha helix (N-3) in the structure, which further packs and stabilizes the hydrophobic core, and further separates the headpiece from its fusion partner. The HP47 tag was added to the amino terminus of the large fragment of Bst-LF, leading to the enzyme denoted herein as Br512 (FIG. 1A). Br512 also contains a N-terminal 8×His-tag for immobilized metal affinity chromatography (IMAC; Ni-NTA). The hypothesis that the villin headpiece could serve as an anchor to improve the folding and/or solubility of Bst-LF proved true, and purification of Br512 proved to be much better than for Bst-LF, ultimately yielding 35 mg of homogenous protein per liter (see also purification protocol).

A cluster of positively charged amino acids are known to be crucial for the actin-binding activity of the headpiece domain (Friederich et al., 1992), and thus another potential advantage of the use of HP47 is that it may allow better interactions with nucleic acid templates (McKnight et al., 1996) (FIG. 1B). In this regard, the HP47 fusion may act similarly to the DNA binding domains used in the construction of synthetic thermostable DNA polymerases that are commonly used for PCR. In the case of Phusion DNA polymerase, the addition of the Sso7d gene, a DNA binding protein from Sulfolobus solfataricus, stabilizes the polymerase/DNA complex and enhances the processivity by up to 9 times, allowing longer amplicons in less time with less influence of PCR inhibitors (Wang et al., 2004). Similarly, fusion of the Bst-like polymerase Gss-polymerase, DNA polymerase I from Geobacillus sp. 777, with DNA binding domains from DNA ligase of Pyrococcus abyssi or Sto7d protein from Sulfolobus tokodaii yielded 3-fold increase in processivity and a 4-fold increase in DNA yield during whole genome amplification (Oscorbin et al., 2017). Whether a similar improvement might prove true for isothermal amplification by Bst DNA polymerase was explored.

### Example 3—Br512 Performs Comparably to Bst 2.0 with DNA Templates

The development of isothermal amplification assays that are both sensitive and robust to sampling is key for continuing to mitigate the ongoing coronavirus pandemic (Esbin et al., 2020). To this end, loop-mediated isothermal amplification has proven to be a useful assay for the detection of SARS-CoV-2 (Park et al., 2020; Huang et al., 2020; Zhang et al., 2020; Yan et al., 2020), including in clinical settings (Dao et al., 2020). However, LAMP is well-known to frequently produce spurious amplicons, even in the absence of template, and thus colorimetric and other methods that do not use sequence-specific probes may be at risk for generating false positive results (Jiang et al., 2015). Therefore, the inventors developed oligonucleotide strand displacement probes that are only triggered in the presence of specific amplicons. These probes are essentially the equivalent of TaqMan probes for qPCR, and can work either in an end-point or continuous fashion with LAMP (Jiang et al., 2015). Base-pairing to the toehold region is extremely sensitive to mismatches, ensuring specificity, and the programmability of both primers and probes makes possible rapid adaptation to the evolution of new SARS-CoV-2 or other disease variants. Higher order molecular information processing is also possible, such as integration of signals from multiple amplicons (Bhadra et al., 2020).

One variant of LAMP, termed LAMP-OSD (for Oligonucleotide Strand Displacement), is designed to be easy to use and interpret, and has been have previously shown to sensitively and reliably detect SARS-CoV-2, including following direct dilution from saliva (Bhadra et al., 2020). Although non-specific signaling of LAMP has been largely mitigated and the assay made more robust for point of need application, the limited choice and supply, and concomitant expense of LAMP enzymes, constitutes a significant roadblock to widespread application of rapid LAMP-based diagnostics. Br512 presents a potential generally available solution to these issues.

To assess whether the engineered changes introduced in Br512 had an impact on enzyme activity, the strand displacing DNA polymerase activity of Br512 was compared with that of the wild type (i.e., parental) Bst-LF enzyme and a commercially sourced engineered Bst-LF with improved amplification speed, Bst 2.0 (New England Biolabs). Duplicate LAMP-OSD assays (Jiang et al., 2015) were set up for the human glyceraldehyde 3-phosphate dehydrogenase (gapd) gene using either 16 units of Bst 2.0 (typical amount used in most LAMP-OSD assays), 20 picomoles (pm) of Bst-LF (a previously optimized amount), or 0.2 pm, 2 μm, 20 μm, or 200 pm of Br512. Real-time measurement of OSD fluorescence revealed that in the presence of 6000 template DNA copies, the DNA polymerase activity of pm of Br512 was comparable to that of 16 units of Bst 2.0 (FIG. 7). The addition of more Br512 did not yield further improvements, although lower amounts reduced the amplification efficiency. In the absence of specific templates, none of the enzymes generated false OSD signals.

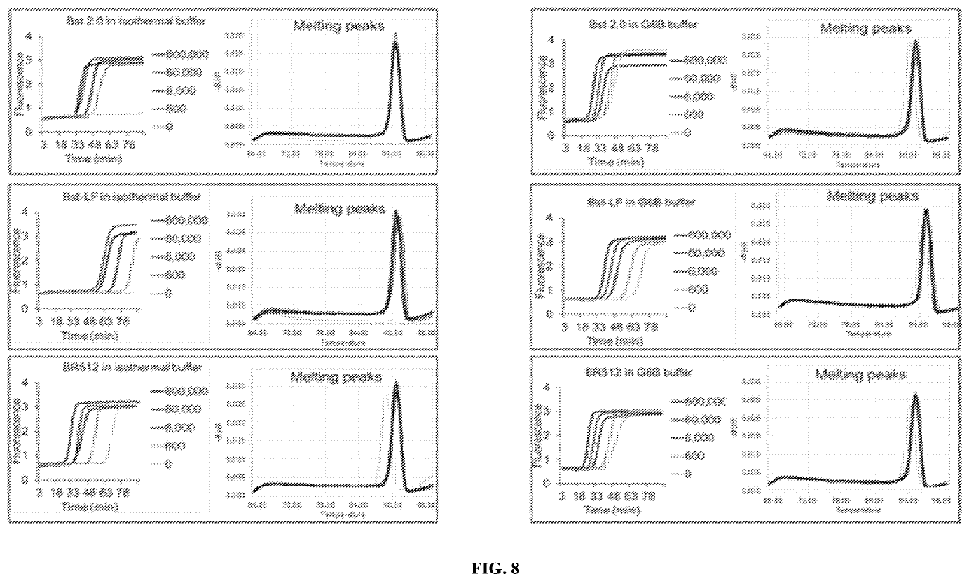

Next, assays were performed with optimized enzyme amounts and either an optimized buffer system, G6 (developed for reverse transcription reactions with 4 mM (B) or 8 mM (D) Mg²⁺), or Isothermal buffer (provided by New England Biolabs). 20 pm of Br512 per LAMP-OSD assay performed comparably to 16 units of Bst 2.0 in terms of both speed and limit of detection (FIGS. 2A,2C,2D). Bst-LF also demonstrated a similar detection limit but with a slower time to signal (FIG. 2B). Similar results were observed via real-time measurements of amplification kinetics using the fluorescent intercalating dye, EvaGreen in place of sequence-specific OSD probes (FIG. 8). Taken together, these results demonstrate that presence of the villin H47 fusion domain in Br512 improves its speed of amplification relative to Bst-LF, making it on par with the DNA amplification speed of Bst 2.0 (New England Biolabs).

### Example 4—Br512 has Superior Performance in Assays with RNA Templates

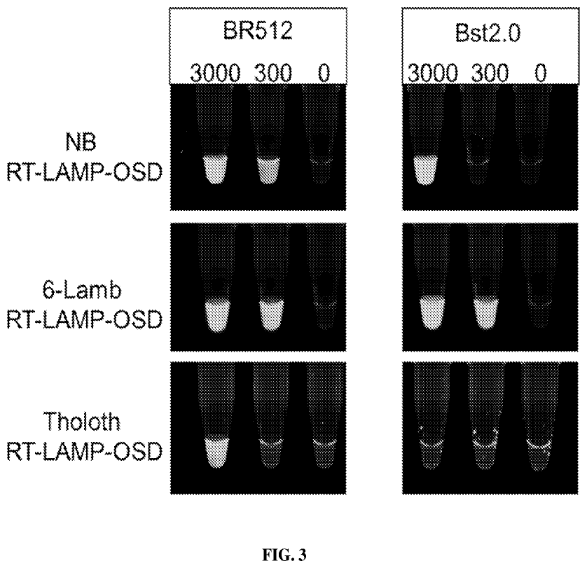

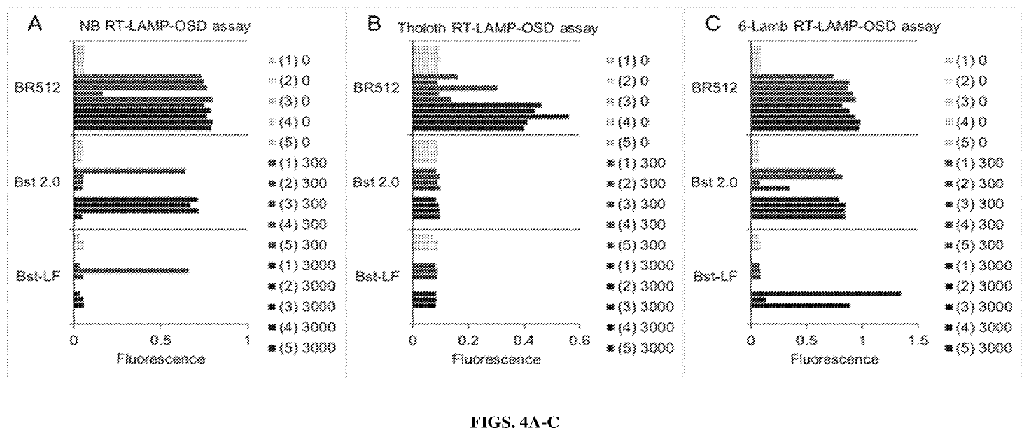

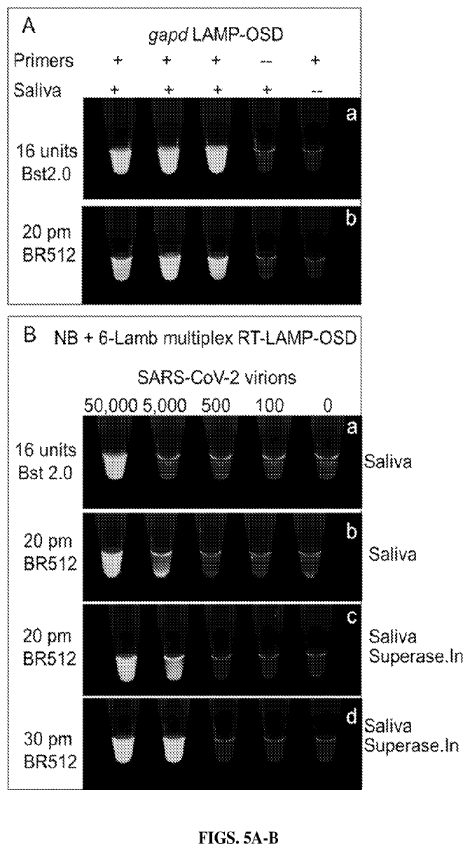

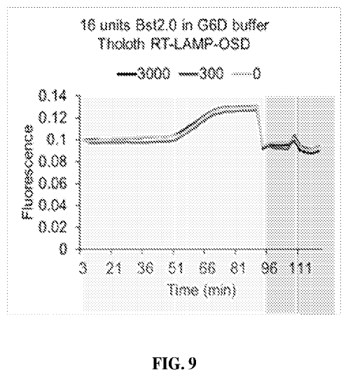

Bst DNA polymerase has been described to possess an inherent reverse transcriptase (RT) activity with chemically diverse nucleic acid templates, including ribonucleic acid (RNA), α-1-threofuranosyl nucleic acid (TNA), and 2′-deoxy-2′-fluoro-β-d-arabino nucleic acid (FANA) (FANA), into DNA (Shi et al., 2015; Jackson et al., 2019). Therefore, the performance of Br512 was tested in RT-LAMP-OSD assays in order to determine whether the engineered enzyme could be used for direct amplification of SARS-CoV-2 RNA. Duplicate reactions were performed with three different primer sets that had previously been shown to work well with LAMP-OSD (termed NB, Tholoth, and 6-Lamb (Bhadra et al., 2020)). These assays were seeded with 3000, 300, or 0 copies of the viral genomic RNA and endpoint OSD fluorescence was imaged following 60 min of amplification at 65° C. All three RT-LAMP-OSD assays performed using Br512 developed bright green OSD fluorescence in the presence of viral genomic RNA indicating successful reverse transcription and LAMP amplification (FIG. 3). While NB and 6-Lamb assays executed with Br512 could readily detect a few hundred viral genomes, the Tholoth assay produced visible signal only with a few thousand viral RNA copies. In contrast, Bst 2.0 demonstrated less robust reverse transcription ability, and failed to generate any OSD signal in the Tholoth assays, both in its companion Isothermal buffer (FIGS. 3 and 4) as well as in the G6D buffer (FIG. 9). Bst 2.0 could reverse transcribe and amplify NB and 6-Lamb sequences resulting in visible OSD fluorescence (FIG. 3), but its detection limit for both assays was higher than with Br512. In all cases, in the absence of primers, reactions remained dark.

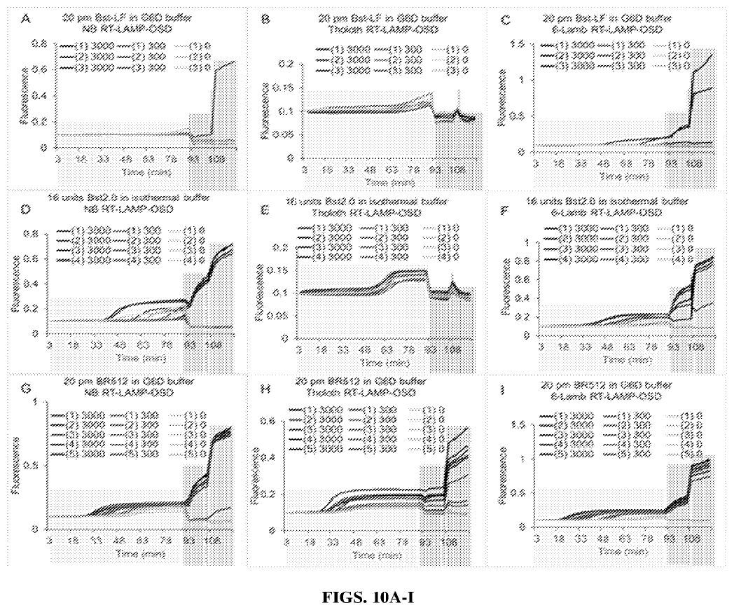

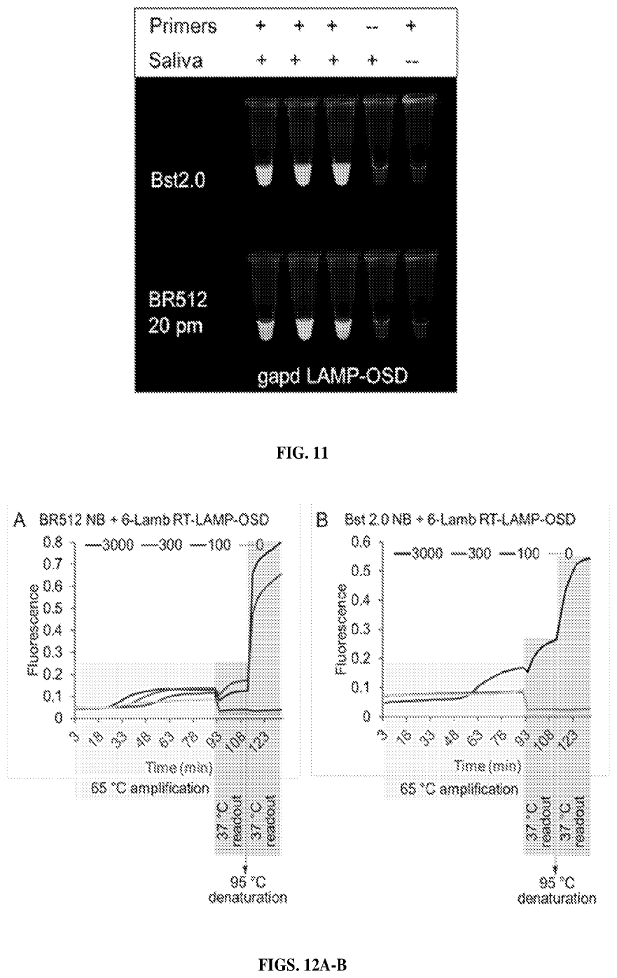

The reliability of detection is an issue, especially at the limit of detection. Br512 could detect 300 SARS-CoV-2 genomes in 80% of NB and 100% of 6-Lamb assays, while Bst 2.0 was successful at detecting this copy number in 25% and 75% of the assays, respectively (FIGS. 4A-C). In addition, Br512 generally demonstrated faster amplification kinetics compared to Bst 2.0 (FIGS. 10A-I). The superiority of Br512 as an enzyme for RT-LAMP was even more significant when compared to Bst-LF. The wild-type, parental enzyme failed to amplify the Tholoth RNA sequence, while only detecting SARS-CoV-2 genomic RNA in 16% of NB assays and 33% of 6-Lamb assays (FIGS. 4A-C). Bst-LF also demonstrated slower amplification kinetics compared to both Br512 and Bst 2.0 (FIGS. 10A-I).

Having shown that individual SARS-CoV-2 amplicons could be successfully generated and detected by Br512 and RT-LAMP-OSD, multiplex assays were set up comprising primers and OSD probes for both the NB and 6-Lamb assays. As with RT-qPCR, such multiplex assays that detect multiple viral genes are of greatest utility for accurate confirmation of the virus (Li et al., 2020; Ishige et al., 2020). When irradiated SARS-CoV-2 virions were added to these assays, Br512 generated distinctly visible OSD signal from as few as 500 virions (FIG. 11A). Duplicate assays executed using Bst 2.0 also produced bright OSD signals from 50,000 virions, while Bst 2.0 assays containing 5000 virions produced a dimmer OSD signal. 500 virions could not be directly detected from saliva (FIG. 11B). The improved detection limit observed for Br512 might be due to the faster kinetics of amplification in multiplex RT-LAMP assays compared to Bst 2.0 (FIG. 12).

### Example 5—Br512 can Detect SARS-CoV-2 Virions in Saliva

LAMP is an appealing technology for rapid point-of-need testing because it does not require thermal cycling, and because the inhibitor tolerance of Bst DNA polymerase can enable direct analysis of clinical and environmental samples, thereby reducing assay complexity (Jiang et al., 2018; Bhadra et al., 2018a; Bhadra et al., 2018b). Previously, highly accurate detection of SARS-CoV-2 virions has been described, including in saliva, using one-pot SARS-CoV-2 RT-LAMP-OSD assays via the standard commercial enzyme mix of Bst 2.0 and RTx (Bhadra et al., 2020).

To further determine whether Br512 can also function in crude clinical specimens, LAMP-OSD assays specific for human gapd sequence were first seeded with human saliva. It should be emphasized that these reactions required no RNA preparation; they were direct dilution assays involving three microliters of saliva pre-heated at 95° C. for 10 min and 22 microliters of LAMP reaction mix. Following 60 min of amplification at 65° C., both Br512 and Bst 2.0 generated bright OSD signals in the presence of saliva indicating successful amplification of endogenous gapd sequences (FIG. 5A). Assays lacking gapd LAMP primers remained as dark as control assays lacking saliva. These results suggest that Br512 is comparable to Bst 2.0 in its ability to amplify endogenous analyte sequences directly from saliva.

When multiplex assays were performed in the presence of 12% (v/v) human saliva in the reactions, both enzymes demonstrated reduced detection ability. In the presence of saliva, Br512 did not detect 500 virions and produced dimmer OSD signal from 5000 virions (FIG. 5Ba). In comparison, Bst 2.0 generated a signal only with 50,000 virions (FIG. 5Bb).

Inclusion of the RNase inhibitor, Superase. In, in the reaction overcame some of the inhibition observed relative to multiplex assays lacking saliva (FIG. 11) and allowed Br512 to produce brighter OSD signal from 5000 virions (FIG. 5Bc). Increasing the amount of Br512 in the reaction further boosted the signal (FIG. 5Bd).

These results demonstrate that it is feasible to conduct direct RNA detection in crude specimens using only Br512, albeit with a potential decline in detection limit. It is also conceivable that with further inhibition mitigation, direct sample analysis with Br512 could approach levels seen with no-prep RT-qPCR with detection limits reported at 6-12 SARS-CoV-2 copies/μL (1,200-2,400 copies/mL) with 5 μL sample volume per reaction for the SalivaDirect protocol implemented at Yale University (Vogels et al., 2020) and 500 to 1000 viral particles/mL with 10 μL sample volume per reaction for the sample prep protocol developed at University of Illinois (Ranoa et al., 2020). In preliminary studies, by including reverse transcriptase enzymes along with Br512, the limit of detection was able to be reduced on the order of 1000 viral particles/mL.

### Example 6—Br512 is Robust to Different Reaction Preparations and Conditions

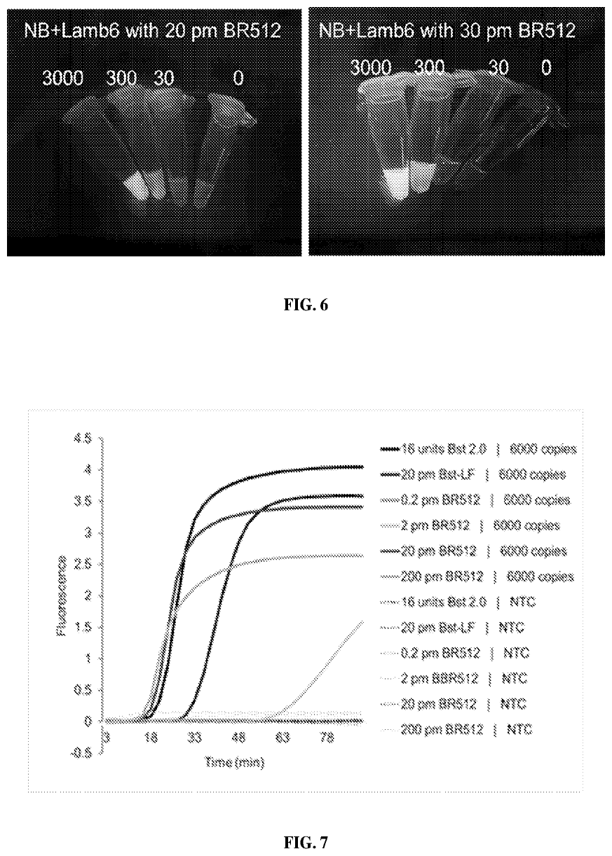

Ready-to-use, freeze-dried assay mixes facilitate large scale distribution and implementation of rapid assays. To determine whether Br512-based single enzyme RT-LAMP-OSD assays might be amenable to drying, master mixes were prepared for the SARS-CoV-2 multiplex assay containing both NB and 6-Lamb primers and OSD probes, along with either 20 pm or 30 pm of Br512, and subjected to lyophilization. After two days, the dry assay pellets were rehydrated and seeded with different amounts of SARS-CoV-2 viral genomic RNA. As shown in FIG. 6, both the 20 μm and 30 pm lyophilized Br512 assays produced visible OSD fluorescence in the presence of as few as 300 viral genomic RNA. Assays lyophilized with more enzyme were generally brighter. These results demonstrate that Br512 is amenable to production of freeze dried single enzyme RT-LAMP assay mixes that are ready for diagnostics simply upon rehydration.

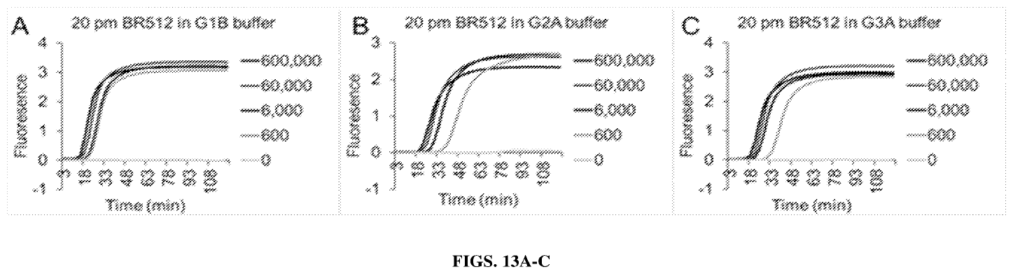

Ideally, diagnostic enzymes should be robust to some variance in reaction conditions, especially those that may be inadvertently introduced by user error or sample variation. The effect of varying buffer and salt concentrations on the activity of BR512 was analyzed with gapd LAMP-OSD reactions. Compared to the optimized G6 buffer previously described, a 66% drop in Tris buffer concentration, 100% decrease or 150% increase in (NH)₂SO₄ amount, or 75% decrease in KCl did not cause significant perturbations in the amplification speed or detection limit of Br512 (FIG. 13). These results suggest that Br512 can perform robustly under varied reaction conditions.

### Example 7—Machine Learning Predictions Improve Br512 Function

Torng and Altman (2017) developed a novel 3D convolutional neural net to examine whether the microenvironments of individual amino acids could be used to classify what amino acids were present throughout a protein's structure. This algorithm trained across the PDB, and in the end was able to re-predict the identity of a given amino acid in a protein with ca. 40% accuracy. This algorithm has since been improved upon by including additional filters for features such as the presence of hydrogen atoms, partial charge, and solvent accessibility, and the accuracy of re-prediction has ultimately improved to upwards of 70% (Shroff et al., 2020). The inventors were interested in the approximately 30% of amino acids that were not predicted to be wild-type; while this might have merely reflected the inaccuracy of the neural network, it was also possible that nature itself was ‘underpredicting’ the fit of a given amino acid to its microenvironment, and that the ensemble of predicted non-wild-type amino acids at a given position might represent opportunities for mutation. To this end, the inventors instantiated the ability to predict underperforming wild-type amino acid residues in proteins by predicting either positions or precise mutations that led to improvements in function across a variety of proteins, including blue fluorescent protein and phosphomannose isomerase (Shroff et al., 2020). It should be noted that such predictions were not for a particular functionality, such as stability or catalysis, but merely for goodness of fit, with improvements in stability and catalysis being empirical outcomes of those predictions.

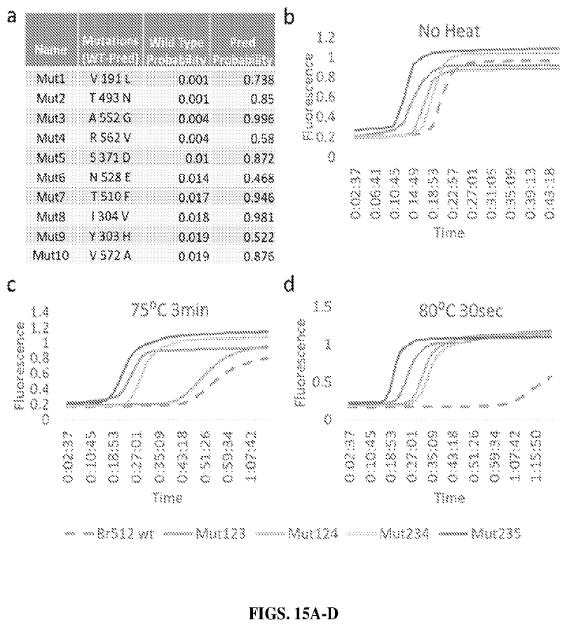

With this track record of success, the inventors attempted to apply the improve algorithm, dubbed MutCompute, to predicting amino acids in the Bst LF that might benefit from mutation. Initially, residues that interacted directly with DNA were filtered from consideration, as the algorithm has not yet been optimized for protein:nucleic acid interactions. An amino acid-by-amino acid assessment of the remaining residues in the protein was carried out, and those positions that revealed the wild-type residue to be the least fit for a given position were called, and ten substitutions were then made that represented the predicted most fit amino acid (FIG. 15A).

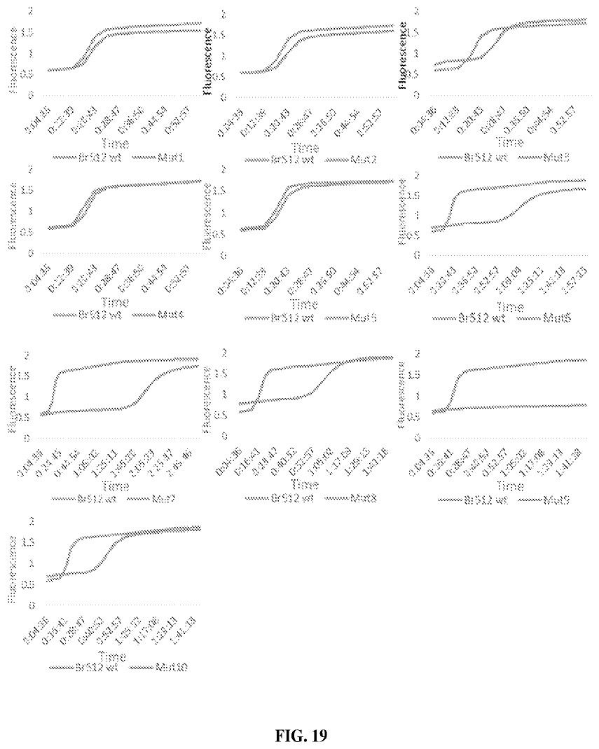

Surprisingly, of the ten amino acid substitutions suggested by MutCompute, only two (Mut6 and Mut9) showed little or no activity in a standard LAMP assay targeting the gene for human GAPDH, while the top 5 (Mutt-5) showed activities as good as or better than the parent enzyme (FIG. 19).

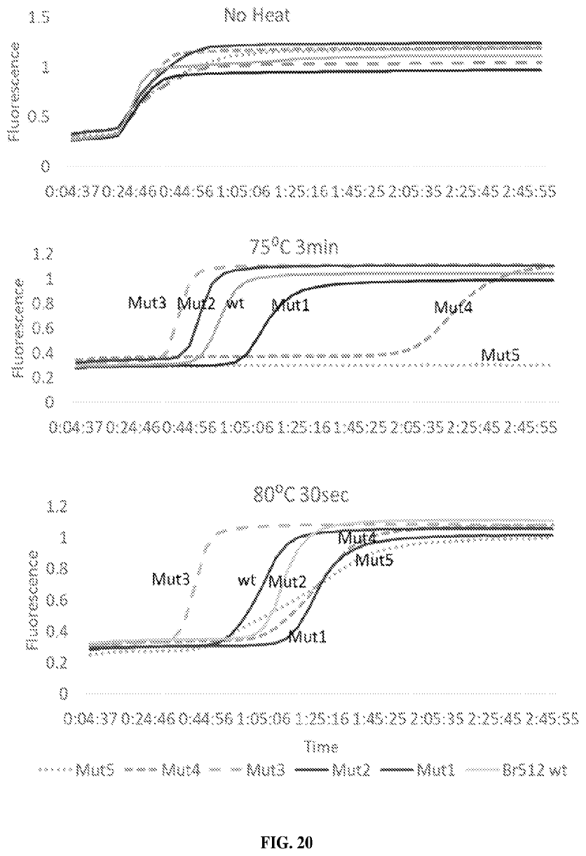

Since the introduction of additive substitutions to achieve higher thermostability was desired, the inventors also adapted LAMP to serve as a simple screen for improved activity at higher temperatures. Initially, enzymes were challenged at temperatures above those typically used for LAMP (75° C. and 80° C.), before carrying out LAMP reactions at their normal temperature (65° C.). Mut1-5 were further assayed with a heat challenge, to determine if they had imparted additional stability to the polymerase (FIG. 20), and both Mut2 and Mut3 were found to be more thermotolerant than the parental enzyme.

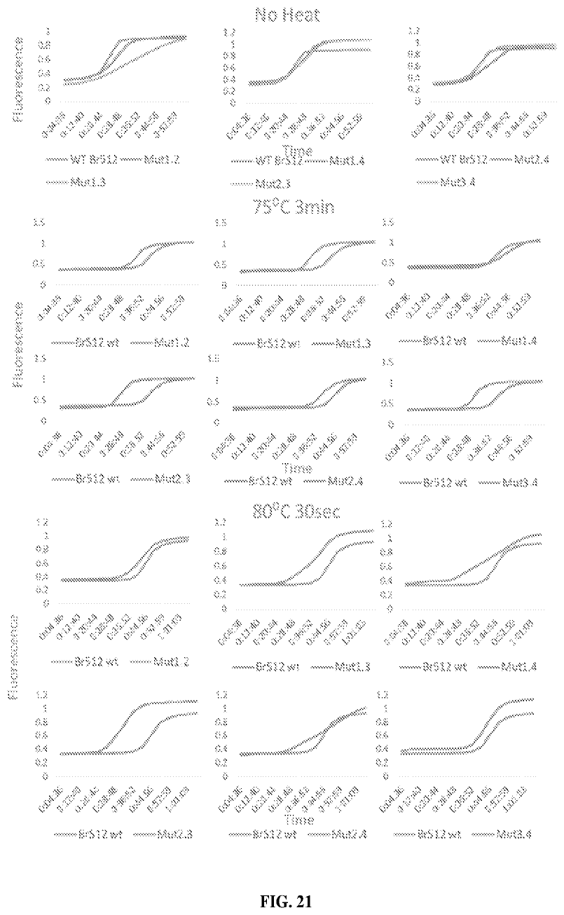

In keeping with the initial hypothesis that different engineering tacks would yield substitutions that had additive impacts on phenotypes, success has previously been had in combining individual mutations predicted via machine-learning approaches to generate proteins with much higher activities; for example, multiple slightly improved variants of blue fluorescent protein could be combined to yield a variant with 5-fold greater fluorescence, while multiple slightly improved variants of a phosphomannose isomerase could be combined to yield a variant with 5-fold greater solubility (Shroff et al., 2020). Therefore, combinations of the point mutations that showed the greatest activity were examined First, all possible double combinations of the Muts 1-4 (Mut12, 13, 14, 23, 24, and 34) were generated and LAMP assays and thermal challenges performed (FIG. 21). Mut23 yielded the most robust activity, in keeping with the results of the initial thermal challenges.

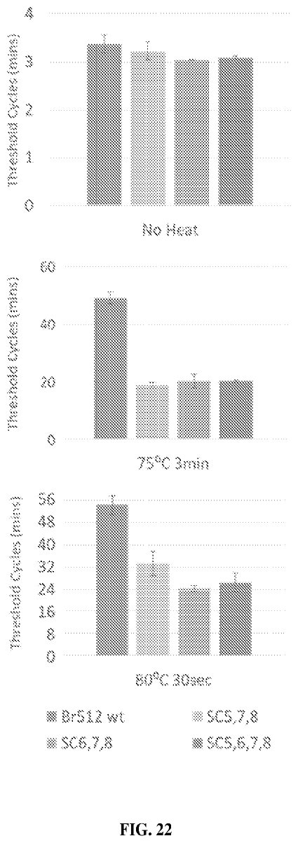

Then, four additional triple mutations (Mut123, Mut124, Mut234, Mut235) that center on Mut23 were generated (FIG. 15). All four triple mutations examined showed robust performance in the normal GAPDH LAMP assay (FIGS. 15B and 22), and the combined machine-learning predicted mutations also displayed strong thermotolerance relative to the parental enzyme, which itself was already superior to Bst-LF (FIGS. 15C, 15D, and 22). Mut235 showed the highest activity, and was therefore used as a platform for further, additive engineering. Interestingly, Mut5 on its own has an inactive phenotype at higher temperatures and seems to serve as a potentiating mutation for additional substitutions.

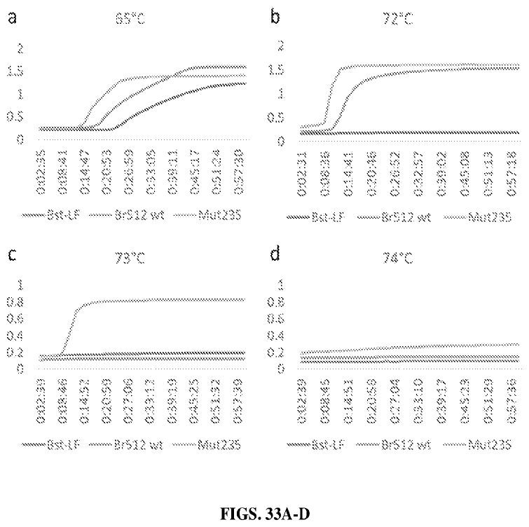

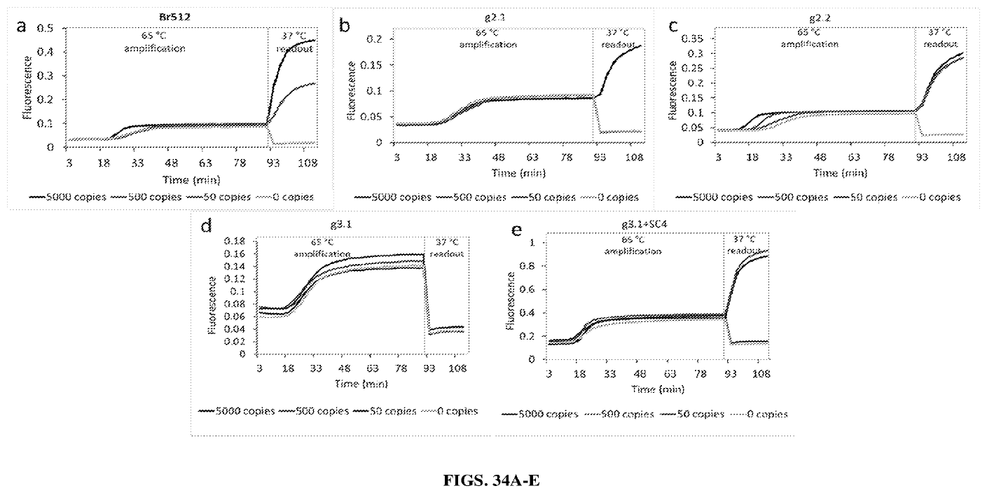

In addition to determining if the substitutions predicted by machine learning approaches would lead to greater thermotolerance, LAMP reactions were attempted at higher temperatures. Surprisingly, the Br512 domain not only improves the performance of Bst DNAP (FIG. 2), improving the time to signal by 6 minutes relative to Bst-LF, but also provides thermostabilization in a LAMP reaction up to 72° C. (FIGS. 33A,33B), where Bst-LF shows no activity. Further increases in performance are provided by the addition of the substitutions predicted by machine learning, with the enzyme now being stable in LAMP reactions up to 73° C. (FIG. 33C), and the overall reaction proceeding 2-4 minutes more quickly than Br512 (FIG. 33A,33B).

While the increased thermostability of the engineered variants was strongly indicated by the thermal challenge assays, a dye-based protein thermal shift assay (TSA) was also performed in order to determine the melting temperatures of the proteins. Br512 showed a slightly higher T m value (76.1° C.) compared to the parental enzyme Bst-LF (75.5° C.), whereas Mut235 demonstrated a greatly improved T m value (78.1° C.), further supporting that the computationally predicted substitutions enhanced thermostability.

### Example 8—Supercharging of the Villin Headpiece (vHP47) Improves Br512 Function

Given that the villin headpiece likely assists with contacting nucleic acid substrates via positively charged patches, the inventors sought to further improve this feature via the addition of excess positively charged amino acids (supercharging; Lawrence et al., 2007). The inventors considered that supercharging could also improve the folding, solubility, and stability of the protein, by decreasing the propensity to aggregate, as previously demonstrated (Simon et al., 2019; Der et al., 2013). It was further anticipated that improvements gained via supercharging would be additive with substitutions introduced via machine-learning approaches, as supercharging targets additional biophysical mechanisms for stabilization, such as potentially improving interactions with the DNA substrate.

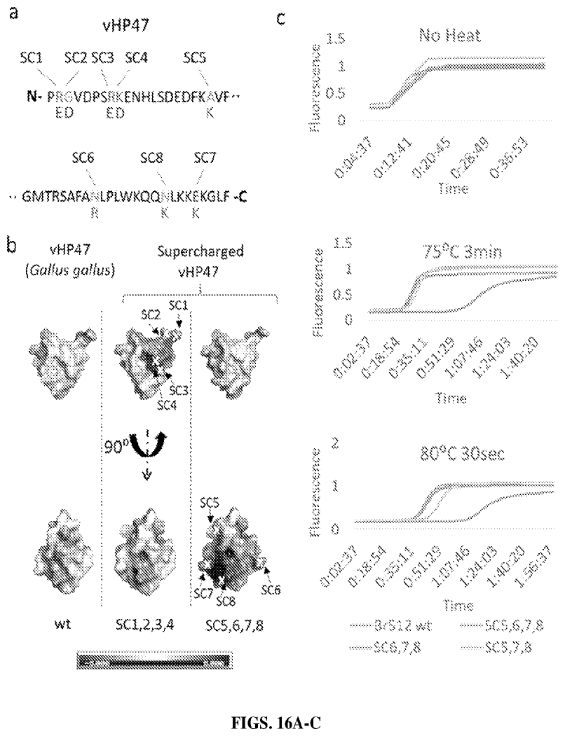

Since vHP47 is naturally a ‘zwitterionic’ or ‘Janus’ protein that contains two oppositely charged surfaces (FIG. 16A), it was hypothesized that it could be further engineered to orient the domain for binding to nucleic acids and thereby potentially enhancing polymerase activity, as previous nucleic acid binding domains (e.g., Phusion) had done. To this end, the inventors initially surveyed highly solvent-exposed amino acids in the vHP47 crystal structure (PDB: 1YU5). A surface charge map generated using the APBS (Adaptive Poisson-Boltzmann Solver) algorithm in Pymol (FIG. 16B) revealed a large cluster of positively charged amino acids on one side of the domain, opposite a smaller cluster of negatively charged amino acids. The positively charged surface might be improving overall DNA-binding, while the negatively charged surface would help with orientation during binding (Marcovitz & Levy, 2011; Lamers et al., 2000).

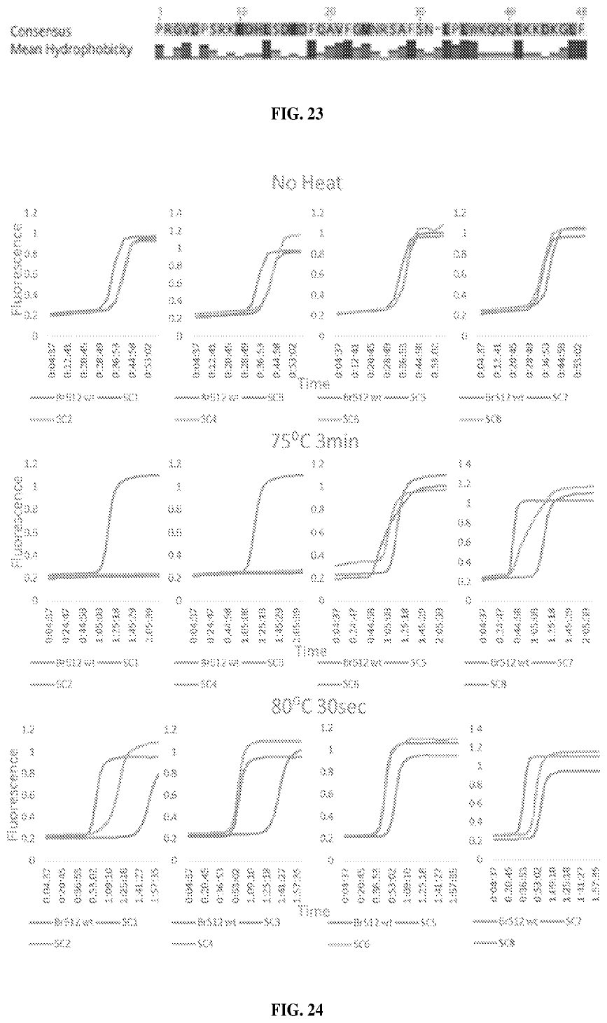

The inventors assessed the relative conservation amongst the top vHP47 100 orthologues from the search (FIG. 22). Variable amino acid positions were selected as candidates for mutagenesis and amino acids that were conserved among HP47 homologues were avoided (FIGS. 16A and 23), and supercharging was focused on single surfaces of vHP47 (FIG. 16B). Eight point mutations (SC1-8; FIG. 16A) were initially engineered. Four of the amino acid substitutions (SC1-4) were designed to enhance the negatively charged surface, while the other four substitutions (SC5-8) were designed to enhance the opposing, positively charged surface (FIG. 16B).

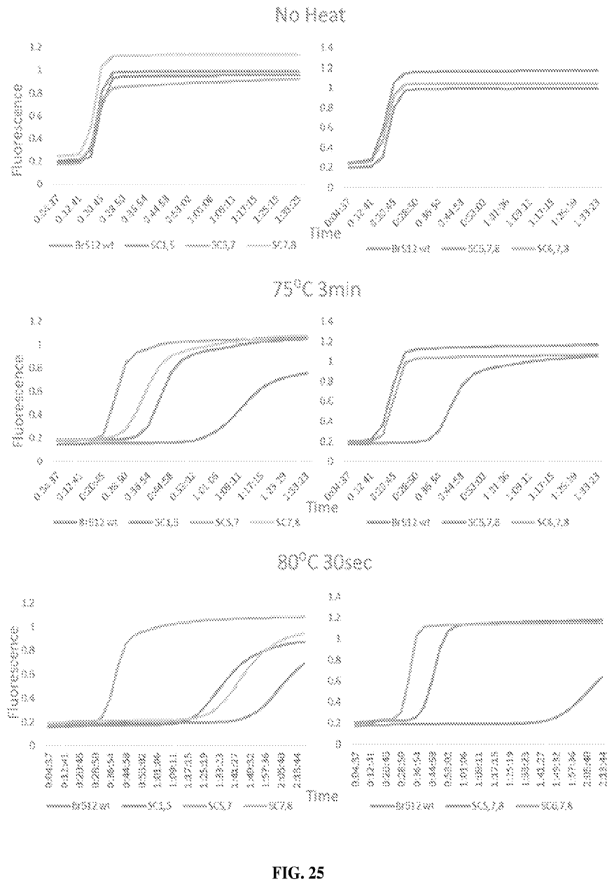

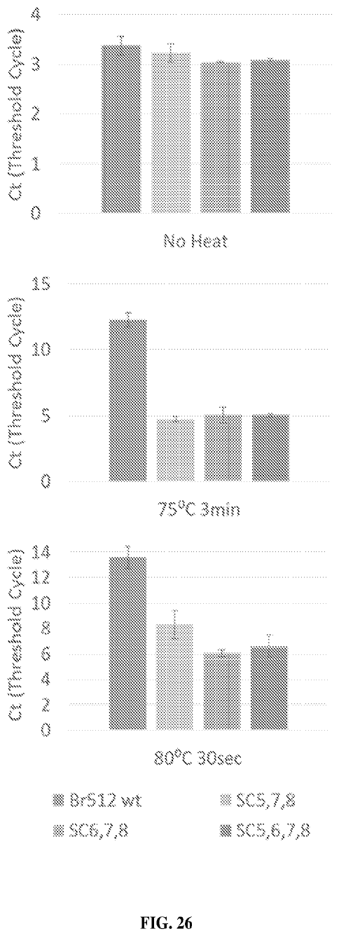

The inventors initially examined the effect of all eight individual charge substitutions (SC1-8) on Br512 enzymatic activity via thermal challenge and subsequent LAMP reactions with GAPDH DNA templates (FIG. 24), as they believed this assay would provide the best grounding for subsequent diagnostics development. While the four, individual, negatively charged substitutions exhibited no enhanced activities, the four positively charged substitutions (SC5, SC6, SC7, SC8) all showed enhanced activities with heat challenges (FIG. 24). To examine the combined effect of the individual substitutions, various double and triple combinations were generated (FIG. 25). The higher order, positively charged substitutions showed roughly additive improvements to heat challenges, suggesting that the increased surface charge imparts either greater overall activity or stability to vHP47 (FIG. 16C; FIGS. 25, 26). The two best variants (SC5,6,7,8 and SC6,7,8) showed robust exponential target amplification starting as early as 18 mins after a heat challenge at 75° C. for three minutes, and at 33 mins after a heat challenge at 80° C. for 0.5 minutes. Both variants were able to amplify targets at least a half an hour earlier than was the case for the wild-type enzyme (Br512) at similar temperatures (FIG. 16C, FIG. 26).

### Example 9—Combining Mutations Further Improves Enzyme Function

As the two independent efforts to engineer the Br512 enzyme resulted in a great success, the inventors sought to examine combinations of the mutations on the Br512. In this regard, the inventors tested the hypothesis that attempts to promote different aspects of enzyme stability (fusion domains, improved amino acid interactions, and supercharging to promote folding and DNA interactions) would operate independently and additively. This hypothesis seemed especially reasonable given that mutations were introduced into separate portions of the Br512 enzyme (Bst-LF and vHP47, respectively).

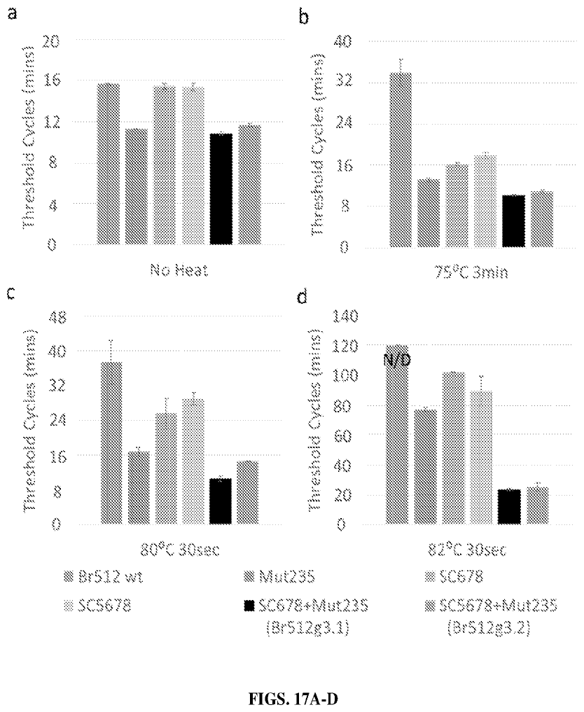

Using the engineered Mut235 variant as a platform, the inventors added the best, combined supercharged mutations (SC678 and SC5678) to generate Mut235-SC678 (also termed Br512g3.1) and Mut235-SC5678 (also termed Br512g3.2) (FIGS. 17A-D). LAMP assays were performed with these two combined variants. But rather than looking at thermal challenge, the inventors further quantitated improvements by looking at the amplification threshold cycle (Cₜ) values, which indicate the time to a significant signal above background. The combined variants had Cₜ values of 10.8 mins, approximately 5 mins faster than the parental Br512 wt (Cₜ of 15.6 mins) even in the absence of a heat challenge prior to LAMP assay (FIG. 17A). This is significant given the need for extremely rapid POC diagnostic assays for consumer and public health applications.

Not only were the combined variants faster, but the combined variants again showed additive and quantitative improvements in the face of heat challenges (FIGS. 17B-D). For instance, the combined variants were faster following a thermal challenge 75° C. for three minutes (10.1 mins) to yield a signal than either of the parental mutations (13 mins and 16.2 mins for Mut235 and SC678, respectively), and at least 24 minutes faster than the parental Br512 enzyme (FIG. 17B). After an 80° C. heat challenge for 30 seconds, the combined variants showed further improvements relative to either the parental mutations (Mut235, SC678, SC5678) or to the parental enzyme (FIG. 17C). Surprisingly, the combined variants Br512g3.1 and Br512g3.2 remained largely active up to 82° C. (FIG. 17D), a temperature that slowed or completely inactivated the parental enzyme.

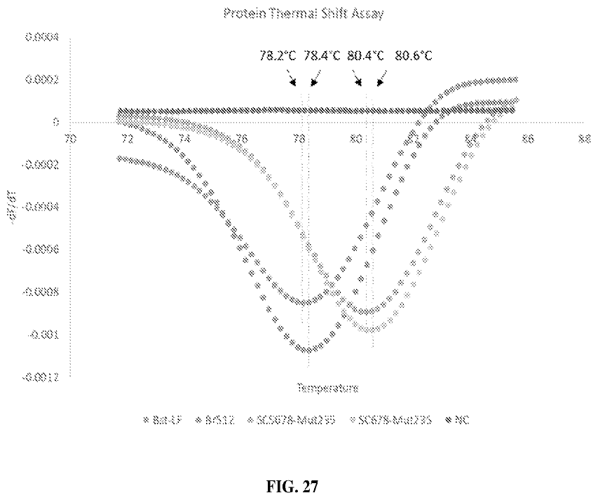

While the increased thermostability of the engineered variants was strongly indicated by the thermal challenge assays, a dye-based protein thermal shift assay (TSA) was also used to determine the melting temperatures of the proteins (FIG. 27). The parental enzyme Br512 showed a slightly higher T m value (78.4° C.) compared to its parental enzyme Bst-LF (78.2° C.), but the two best variants showed greatly improved T m values: 80.4 C and 80.6° C. for SC5678-Mut235 (also termed Br512g3.1) and SC678-Mut235 (also termed Br512g3.2), respectively, further supporting the enhanced thermostability of the engineered variants.

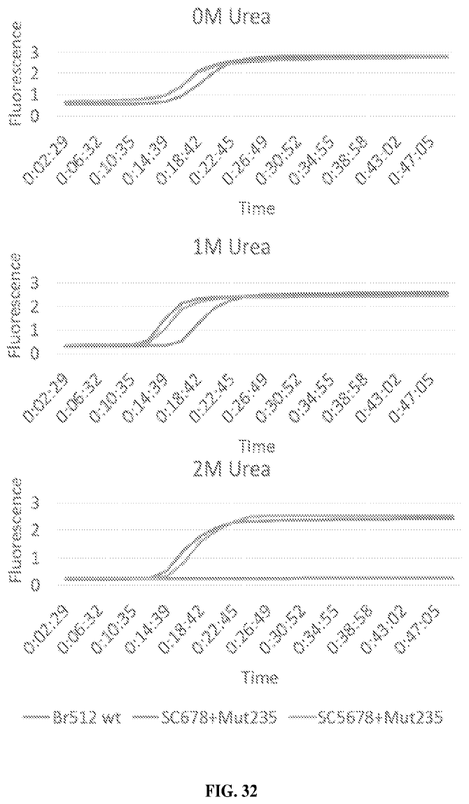

The fact that thermostability in LAMP was underpinned by overall structural stability, as indicated by the thermal shift assay, suggested that the enzyme might be generally more robust to structural perturbations. Therefore, whether the enzymes could survive treatments with chaotropes was determined. This was especially important, since guanidinium is used in many viral inactivation media, and urea is a major component in urine samples, inhibiting PCR reactions at or above a concentration of 50 mM (Khan et al., 1991). Therefore, the activities of Br512g3 was examined in the GAPDH DNA LAMP assay in the presence of varying amounts (0-2 M final concentrations) of the chaotropic agent urea (FIG. 32). While the parental enzyme was completely inactivated at 2 M urea concentrations, the Br512g3 enzymes successfully amplified the target GAPDH DNA. Interestingly, we observed exponential amplifications starting at −10 min with g3.1 and g3.2 variants even in the presence of 1M urea (FIG. 32), which is about 4 min faster than LAMP performed without chaotropes (FIG. 32). This is consistent with the fact that temperature can improve strand separation, and hence the speed of LAMP, and with previous data that shows that urea can increase the sensitivity of LAMP (Cai et al., 2018). This observation opens the way to not only high temperature LAMP, but LAMP that requires little or no sample preparation (as has previously been shown with saliva (Du et al., 2017; Du et al., 2015)), including direct dilution from viral inactivation media, which contains substantial concentrations of guanidinium.

### Example 10—Combined Variants Allow Extraordinarily Fast LAMP Reactions

LAMP and other isothermal amplification methods rely on a relatively small number of DNA polymerases with unique strand-displacement properties (Chader et al., 2014; Oscorbin et al., 2017; Notomi et al., 2000). The overall speed of LAMP reactions is generally limited by the strand displacement ability of the enzyme, and by the related ability of the amplified DNA to form single-strands that can fold back and create a new 3′ hairpin in the growing concatemers. Given the increases in speed observed during thermal challenges, it seemed possible that an optimal temperature could be identified for ultrafast amplification, where the strand displacement capabilities of the combined variants would be optimally balanced with the ability to bind primers and form new 3′ hairpins.

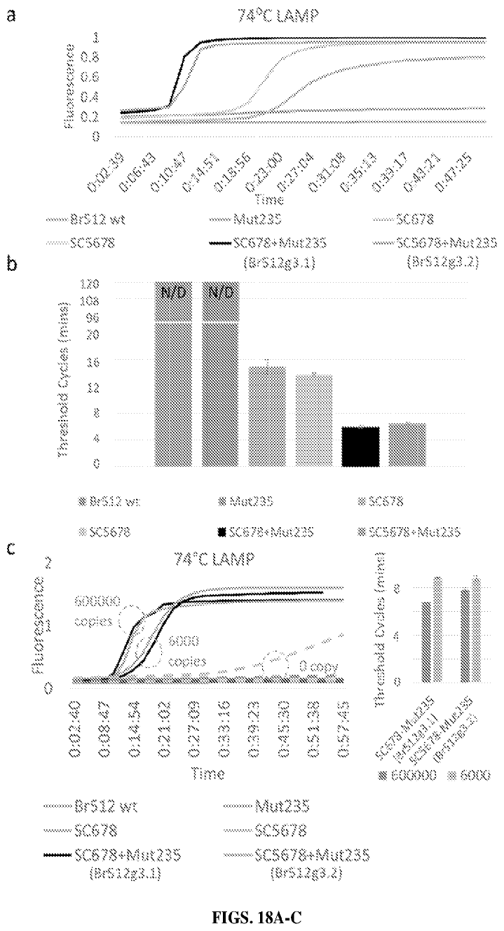

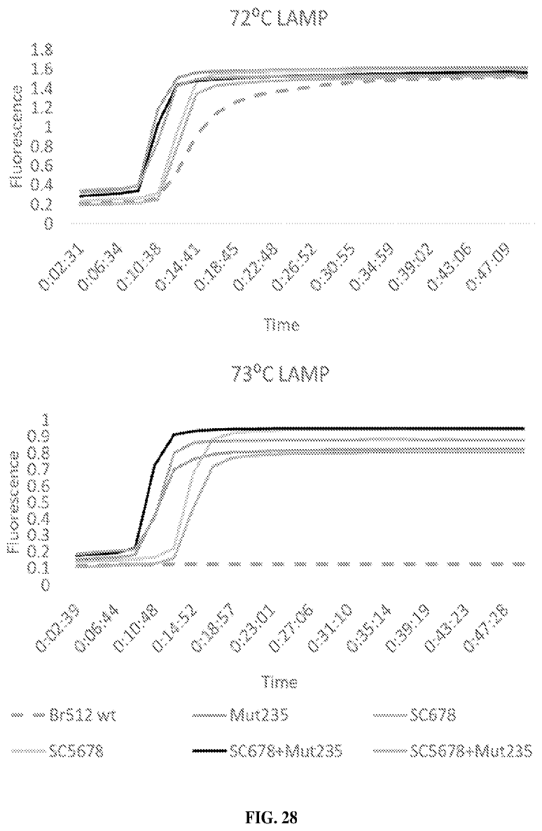

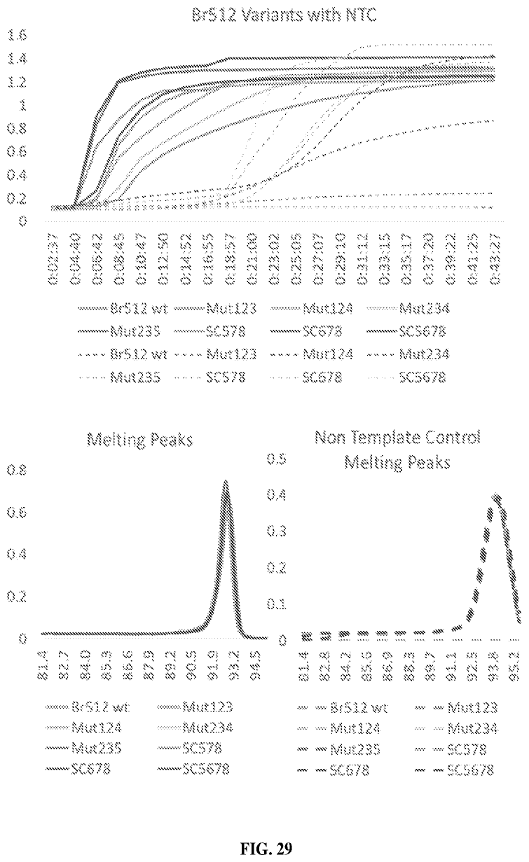

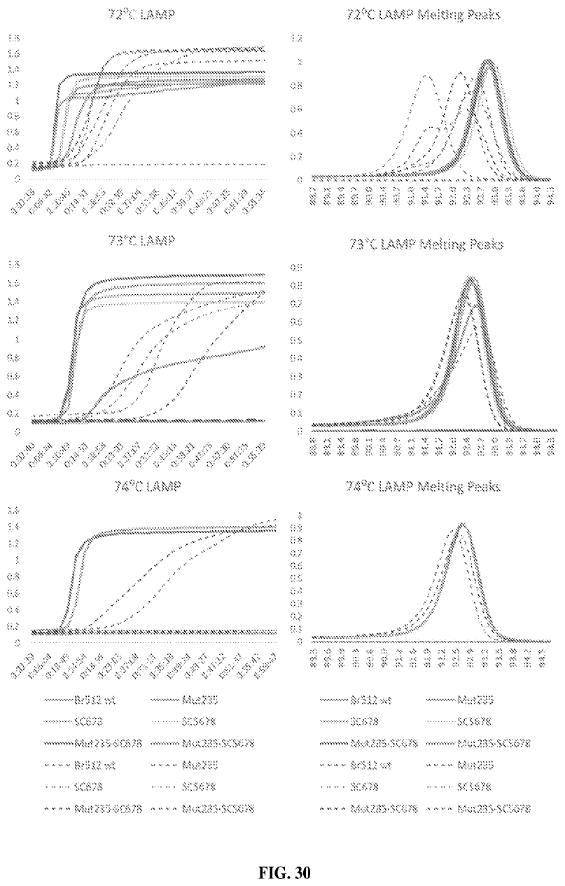

By scanning through several temperatures (FIG. 28), it was found that the LAMP reaction could enter into a readily detected exponential phase as soon as 6 minutes, with the overall reaction being completed by ˜10 min at 74° C. LAMP (FIG. 18A). Quantitation by time to detection (threshold cycle) showed that the Cₜ value of the variants was as low as 6 mins (FIG. 18B). Overall, the time to detection (reaction time to reach Cₜ) for 6×10⁷ copies of GAPDH DNA templates was ˜6 min (FIG. 18A) and ˜7 min for Br512g3.1 and Br512g3.2, respectively for 600,000 copies (FIG. 18C), and ˜9 min for 6,000 copies (FIG. 18C). In contrast, the parental Br512 enzyme was completely inactivated in continuous amplification assays (as opposed to thermal challenges) at 73° C. and 74° C. (FIGS. 28, 18A, 18B, and 18C). The specificities of the GAPDH LAMP assays shown in this study were verified with non-template controls (FIGS. 29 and 30). In general, the engineered enzymes are more thermotolerant to brief challenges than to continuous function at high temperatures; for example, enzymes that are thermotolerant for short periods of time at 75° C. or 80° C. may not show activity in LAMP at 74° C.

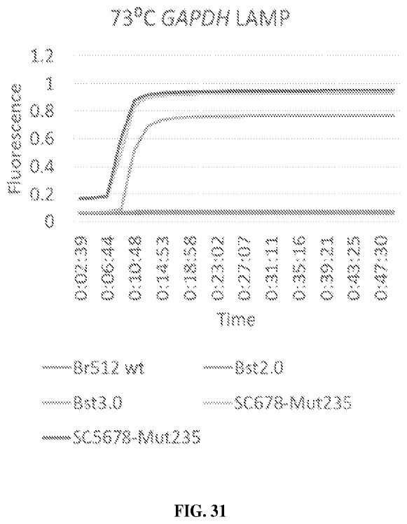

Finally, the inventors performed a 73° C. LAMP assay using commercially available Bst2.0 and Bst3.0 enzymes (from NEB) with the buffer (Isothermal II buffer) provided by the manufacturer. The top two variants outperformed Bst2.0 and Bst3.0 polymerases in the GAPDH LAMP assay (FIG. 31), even in the alternative buffer. While Bst2.0 was completely inactive, Bst3.0 was slower to produce a signal than the top two variants (Br512g3.1 and Br512g3.2) by about 2 mins.

### Example 11—Modulating Charge Impacts Relative Polymerase Activities

While the efforts to engineer Br512 resulted in greatly improved stabilities and activities, the inventors unexpectedly noticed that the inherent reverse transcription activity of Bst-LF was reduced in the engineered enzymes (FIGS. 34A-E). While the parental Br512 still showed considerable reverse transcription activity in NB RT-LAMP-OSD assays (FIG. 34A), and could detect as low as 500 copies of SARS-Cov-2 RNA templates, the g3.1 variant showed reduced or sometimes no reverse transcription activity (FIG. 34D). To dissect the underlying cause of RT inhibition, the inventors also carried out RT-LAMP-OSD assays using either only the charge-engineered variant (g2.1; SC6,7,8) or only the machine learning-engineered variant (g2.2; Mut2,3,5) (FIGS. 34B,C). While the machine learning-engineered variant still exhibited robust reverse transcription activity (FIG. 34C), the charge-engineered variant (g2.1) showed reduced reverse transcription activity (FIG. 34B).

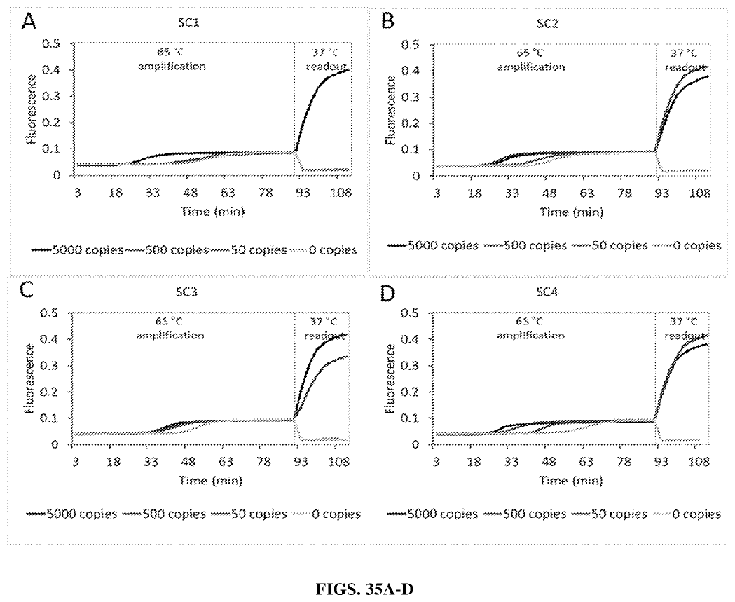

Since the charge engineering effort added positive charges only to one face of the Janus-like HP47 (FIG. 16B), the inventors speculated that this may have changed interaction kinetics between the HP47 and nucleic acids in a way that reduced reverse transcription activity. Thus, in an attempt to restore RT activity while retaining higher stability and activity, the inventors attempted to charge balance the HP47 surface by adding additional, negative substitutions on the opposite face. While the four, previously identified negative substitutions did not show any altered reverse transcription activities compared to their parental Br512 enzyme (FIGS. 35A-D), when SC4 was introduced to the combined variant g3.1 it largely restored reverse transcription activity (FIG. 35E). These results suggest that the charge balance of the anchor domain is a further parameter that can be modulated to fine-tune the activities of polymerases.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aliotta, J. M., Pelletier, J. J., Ware, J. L., Moran, L. S., Benner, J. S., and Kong, H. (1996) Thermostable Bst DNA polymerase I lacks a 3′-->5′ proofreading exonuclease activity, Genet Anal 12, 185-195.
Alley, E. C., Khimulya, G., Biswas, S., AlQuraishi, M., and Church, G. M. (2019) Unified rational protein engineering with sequence-based deep representation learning, Nature methods 16, 1315-1322.
Banach, M., Stapor, K., Konieczny, L., Fabian, P. and Roterman, I. (2020) Downhill, Ultrafast and Fast Folding Proteins Revised. Int J Mol Sci, 21.
Bazari, W. L., Matsudaira, P., Wallek, M., Smeal, T., Jakes, R. and Ahmed, Y. (1988) Villin sequence and peptide map identify six homologous domains. Proceedings of the National Academy of Sciences of the United States of America, 85, 4986-4990.
Bhadra, S., Maranhao, A. C. and Ellington, A. D. (2020) One enzyme reverse transcription qPCR using Taq DNA polymerase. bioRxiv, 2020.2005.2027.120238.
Bhadra, S., Riedel, T. E., Saldana, M. A., Hegde, S., Pederson, N., Hughes, G. L. and Ellington, A. D. (2018a) Direct nucleic acid analysis of mosquitoes for high fidelity species identification and detection of Wolbachia using a cellphone. PLoS Negl Trop Dis, 12, e0006671.
Bhadra, S., Saldana, M. A., Han, H. G., Hughes, G. L. and Ellington, A. D. (2018b) Simultaneous Detection of Different Zika Virus Lineages via Molecular Computation in a Point-of-Care Assay. Viruses, 10, Preprint accessible at: doi: https://doi.org/10.1101/424440.
Bhadra, S., Riedel, T. E., Lakhotia, S., Tran, N. D. and Ellington, A. D. (2020) High-surety isothermal amplification and detection of SARS-CoV-2, including with crude enzymes. bioRxiv, 2020.2004.2013.039941.
Bi, Y., Tang, Y., Raleigh, D. P. and Cho, J. H. (2006) Efficient high level expression of peptides and proteins as fusion proteins with the N-terminal domain of L9: application to the villin headpiece helical subdomain. Protein Expr Purif, 47, 234-240.
Cai, S., Jung, C., Bhadra, S. and Ellington, A. D. (2018) Phosphorothioated Primers Lead to Loop-Mediated Isothermal Amplification at Low Temperatures. Anal Chem, 90, 8290-8294.
Chander, Y., Koelbl, J., Puckett, J., Moser, M. J., Klingele, A. J., Liles, M. R., Carrias, A., Mead, D. A. and Schoenfeld, T. W. (2014) A novel thermostable polymerase for RNA and DNA loop-mediated isothermal amplification (LAMP). Front Microbiol, 5, 395.
Chiu, T. K., Kubelka, J., Herbst-Irmer, R., Eaton, W. A., Hofrichter, J. and Davies, D. R. (2005) High-resolution x-ray crystal structures of the villin headpiece subdomain, an ultrafast folding protein. Proceedings of the National Academy of Sciences of the United States of America, 102, 7517-7522.
Coulther, T. A., Stern, H. R., and Beuning, P. J. (2019) Engineering Polymerases for New Functions, Trends Biotechnol 37, 1091-1103.
Dao Thi, V. L., Herbst, K., Boerner, K., Meurer, M., Kremer, L. P., Kirrmaier, D., Freistaedter, A., Papagiannidis, D., Galmozzi, C., Stanifer, M. L. et al. (2020) A colorimetric RT-LAMP assay and LAMP-sequencing for detecting SARS-CoV-2 RNA in clinical samples. Sci Transl Med, 12.
Der, B. S., Kluwe, C., Miklos, A. E., Jacak, R., Lyskov, S., Gray, J. J., Georgiou, G., Ellington, A. D. and Kuhlman, B. (2013) Alternative computational protocols for supercharging protein surfaces for reversible unfolding and retention of stability. PloS one, 8, e64363.
Du, Y., Pothukuchy, A., Gollihar, J. D., Nourani, A., Li, B. and Ellington, A. D. (2017) Coupling Sensitive Nucleic Acid Amplification with Commercial Pregnancy Test Strips. Angew Chem Int Ed Engl, 56, 992-996.
Du, Y., Hughes, R. A., Bhadra, S., Jiang, Y. S., Ellington, A. D. and Li, B. (2015) A Sweet Spot for Molecular Diagnostics: Coupling Isothermal Amplification and Strand Exchange Circuits to Glucometers. Sci Rep, 5, 11039.
Esbin, M. N., Whitney, O. N., Chong, S., Maurer, A., Darzacq, X. and Tjian, R. (2020) Overcoming the bottleneck to widespread testing: a rapid review of nucleic acid testing approaches for COVID-19 detection. RNA, 26, 771-783.
Flores, H. and Ellington, A. D. (2002) Increasing the thermal stability of an oligomeric protein, beta-glucuronidase. J Mol Biol, 315, 325-337.
Folkman, L., Stantic, B., Sattar, A., and Zhou, Y. (2016) EASE-MM: Sequence-Based Prediction of Mutation-Induced Stability Changes with Feature-Based Multiple Models, Journal of molecular biology 428, 1394-1405.
Fox, J. D., Routzahn, K. M., Bucher, M. H. and Waugh, D. S. (2003) Maltodextrin-binding proteins from diverse bacteria and archaea are potent solubility enhancers. FEBS Lett, 537, 53-57.
Friederich, E., Vancompernolle, K., Huet, C., Goethals, M., Finidori, J., Vandekerckhove, J. and Louvard, D. (1992) An actin-binding site containing a conserved motif of charged amino acid residues is essential for the morphogenic effect of villin. Cell, 70, 81-92.
Hsieh, K., Mage, P. L., Csordas, A. T., Eisenstein, M. and Soh, H. T. (2014) Simultaneous elimination of carryover contamination and detection of DNA with uracil-DNA-glycosylase-supplemented loop-mediated isothermal amplification (UDG-LAMP). Chem Commun (Camb), 50, 3747-3749.
Huang, W. E., Lim, B., Hsu, C. C., Xiong, D., Wu, W., Yu, Y., Jia, H., Wang, Y., Zeng, Y., Ji, M. et al. (2020) RT-LAMP for rapid diagnosis of coronavirus SARS-CoV-2. Microb Biotechnol, 13, 950-961.
Ishige, T., Murata, S., Taniguchi, T., Miyabe, A., Kitamura, K., Kawasaki, K., Nishimura, M., Igari, H. and Matsushita, K. (2020) Highly sensitive detection of SARS-CoV-2 RNA by multiplex rRT-PCR for molecular diagnosis of COVID-19 by clinical laboratories. Clin. Chim Acta, 507, 139-142.
Ishino, S., and Ishino, Y. (2014) DNA polymerases as useful reagents for biotechnology—the history of developmental research in the field, Front Microbiol 5, 465.
Jackson, L. N., Chim, N., Shi, C. and Chaput, J. C. (2019) Crystal structures of a natural DNA polymerase that functions as an XNA reverse transcriptase. Nucleic Acids Res, 47, 6973-6983.
Jiang, Y. S., Bhadra, S., Li, B., Wu, Y. R., Milligan, J. N. and Ellington, A. D. (2015) Robust strand exchange reactions for the sequence-specific, real-time detection of nucleic acid amplicons. Anal Chem, 87, 3314-3320.
Jiang, Y. S., Riedel, T. E., Popoola, J. A., Morrow, B. R., Cai, S., Ellington, A. D. and Bhadra, S. (2018) Portable platform for rapid in-field identification of human fecal pollution in water. Water Res, 131, 186-195.
Kapust, R. B. and Waugh, D. S. (1999) Escherichia coli maltose-binding protein is uncommonly effective at promoting the solubility of polypeptides to which it is fused. Protein Sci, 8, 1668-1674.
Khan, G., Kangro, H. O., Coates, P. J. and Heath, R. B. (1991) Inhibitory effects of urine on the polymerase chain reaction for cytomegalovirus DNA. J Clin Pathol, 44, 360-365.
Koskinen, P., Toronen, P., Nokso-Koivisto, J., and Holm, L. (2015) PANNZER: high-throughput functional annotation of uncharacterized proteins in an error-prone environment, Bioinformatics 31, 1544-1552.
Krishna, M. M. and Englander, S. W. (2005) The N-terminal to C-terminal motif in protein folding and function. Proceedings of the National Academy of Sciences of the United States of America, 102, 1053-1058.
Lamers, M. H., Perrakis, A., Enzlin, J. H., Winterwerp, H. H., de Wind, N. and Sixma, T. K. (2000) The crystal structure of DNA mismatch repair protein MutS binding to a G×T mismatch. Nature, 407, 711-717.
Lawrence, M. S., Phillips, K. J. and Liu, D. R. (2007) Supercharging proteins can impart unusual resilience. Journal of the American Chemical Society, 129, 10110-10112.
Lawyer, F. C., Stoffel, S., Saiki, R. K., Chang, S. Y., Landre, P. A., Abramson, R. D. and Gelfand, D. H. (1993) High-level expression, purification, and enzymatic characterization of full-length Thermus aquaticus DNA polymerase and a truncated form deficient in 5′ to 3′ exonuclease activity. PCR methods and applications, 2, 275-287.
Lei, H., Wu, C., Liu, H. and Duan, Y. (2007) Folding free-energy landscape of villin headpiece subdomain from molecular dynamics simulations. Proceedings of the National Academy of Sciences of the United States of America, 104, 4925-4930.
Li, C., Debruyne, D. N., Spencer, J., Kapoor, V., Liu, L. Y., Zhou, B., Lee, L., Feigelman, R., Burdon, G., Liu, J. et al. (2020) High sensitivity detection of coronavirus SARS-CoV-2 using multiplex PCR and a multiplex-PCR-based metagenomic method. bioRxiv, 2020.2003.2012.988246.
Ma, Y., Zhang, B., Wang, M., Ou, Y., Wang, J., and Li, S. (2016) Enhancement of Polymerase Activity of the Large Fragment in DNA Polymerase I from Geobacillus stearothermophilus by Site-Directed Mutagenesis at the Active Site, BioMed research international 2016, 2906484.
Marcovitz, A. and Levy, Y. (2011) Frustration in protein-DNA binding influences conformational switching and target search kinetics. Proc Natl Acad Sci USA, 108, 17957-17962.
Matsumura, I., Wallingford, J. B., Surana, N. K., Vize, P. D. and Ellington, A. D. (1999) Directed evolution of the surface chemistry of the reporter enzyme beta-glucuronidase. Nat Biotechnol, 17, 696-701.
McKnight, C. J., Doering, D. S., Matsudaira, P. T. and Kim, P. S. (1996) A thermostable 35-residue subdomain within villin headpiece. Journal of molecular biology, 260, 126-134.
Miklos, A. E., Kluwe, C., Der, B. S., Pai, S., Sircar, A., Hughes, R. A., Berrondo, M., Xu, J., Codrea, V., Buckley, P. E. et al. (2012) Structure-based design of supercharged, highly thermoresistant antibodies. Chem Biol, 19, 449-455.
Mildvan, A. S., Weber, D. J. and Kuliopulos, A. (1992) Quantitative interpretations of double mutations of enzymes. Archives of biochemistry and biophysics, 294, 327-340.
Mildvan, A. S. (2004) Inverse thinking about double mutants of enzymes. Biochemistry, 43, 14517-14520.
Naganathan, A. N. and Munoz, V. (2005) Scaling of folding times with protein size. Journal of the American Chemical Society, 127, 480-481.
Nazina, T. N., Tourova, T. P., Poltaraus, A. B., Novikova, E. V., Grigoryan, A. A., Ivanova, A. E., Lysenko, A. M., Petrunyaka, V. V., Osipov, G. A., Belyaev, S. S., and Ivanov, M. V. (2001) Taxonomic study of aerobic thermophilic bacilli: descriptions of Geobacillus subterraneus gen. nov., sp. nov. and Geobacillus uzenensis sp. nov. from petroleum reservoirs and transfer of Bacillus stearothermophilus, Bacillus thermocatenulatus, Bacillus thermoleovorans, Bacillus kaustophilus, Bacillus thermodenitrificans to Geobacillus as the new combinations G. stearothermophilus, G. th, International journal of systematic and evolutionary microbiology 51, 433-446.
Nikoomanzar, A., Chim, N., Yik, E. J. and Chaput, J. C. (2020) Engineering polymerases for applications in synthetic biology. Quarterly reviews of biophysics, 53, e8.
Notomi, T., Okayama, H., Masubuchi, H., Yonekawa, T., Watanabe, K., Amino, N. and Hase, T. (2000) Loop-mediated isothermal amplification of DNA. Nucleic acids research, 28, E63.
Oscorbin, I. P., Belousova, E. A., Boyarskikh, U. A., Zakabunin, A. I., Khrapov, E. A. and Filipenko, M. L. (2017) Derivatives of Bst-like Gss-polymerase with improved processivity and inhibitor tolerance. Nucleic Acids Res, 45, 9595-9610.
Oskarsson, K. R., Saevarsson, A. F. and Kristjansson, M. M. (2020) Thermostabilization of VPR, a kinetically stable cold adapted subtilase, via multiple proline substitutions into surface loops. Scientific reports, 10, 1045.
Panno, S., Matic, S., Tiberini, A., Caruso, A. G., Bella, P., Torta, L., Stassi, R., and Davino, A. S. (2020) Loop Mediated Isothermal Amplification: Principles and Applications in Plant Virology, Plants (Basel) 9.
Park, G. S., Ku, K., Baek, S. H., Kim, S. J., Kim, S. I., Kim, B. T. and Maeng, J. S. (2020) Development of Reverse Transcription Loop-Mediated Isothermal Amplification Assays Targeting Severe Acute Respiratory Syndrome Coronavirus 2 (SARS-CoV-2). The Journal of molecular diagnostics: JMD, 22, 729-735.
Pavlov, A. R., Pavlova, N. V., Kozyavkin, S. A., and Slesarev, A. I. (2012) Cooperation between Catalytic and DNA Binding Domains Enhances Thermostability and Supports DNA Synthesis at Higher Temperatures by Thermostable DNA Polymerases, Biochemistry 51, 2032-2043.
Pinheiro, V. B. (2019) Engineering-driven biological insights into DNA polymerase mechanism. Current opinion in biotechnology, 60, 9-16.
Piotrowski, Y., Gurung, M. K., and Larsen, A. N. (2019) Characterization and engineering of a DNA polymerase reveals a single amino-acid substitution in the fingers subdomain to increase strand-displacement activity of A-family prokaryotic DNA polymerases, Bmc Mol Cell Biol 20.
Powell, K. A., Ramer, S. W., Del Cardayre, S. B., Stemmer, W. P., Tobin, M. B., Longchamp, P. F. and Huisman, G. W. (2001) Directed Evolution and Biocatalysis. Angewandte Chemie, 40, 3948-3959.
Ranoa, D. R. E., Holland, R. L., Alnaji, F. G., Green, K. J., Wang, L., Brooke, C. B., Burke, M. D., Fan, T. M. and Hergenrother, P. J. (2020) Saliva-Based Molecular Testing for SARS-CoV-2 that Bypasses RNA Extraction. bioRxiv, 2020.2006.2018.159434.
Rao, R., Bhattacharya, N., Thomas, N., Duan, Y., Chen, X., Canny, J., Abbeel, P., and Song, Y. S. (2019) Evaluating Protein Transfer Learning with TAPE, Adv Neural Inf Process Syst 32, 9689-9701.
Richard, J., Kim, E. D., Nguyen, H., Kim, C. D. and Kim, S. (2016) Allostery Wiring Map for Kinesin Energy Transduction and Its Evolution. The Journal of biological chemistry, 291, 20932-20945.
Romero, P. A., Krause, A., and Arnold, F. H. (2013) Navigating the protein fitness landscape with Gaussian processes, Proceedings of the National Academy of Sciences of the United States of America 110, E193-201.
Saito, Y., Oikawa, M., Nakazawa, H., Niide, T., Kameda, T., Tsuda, K., and Umetsu, M. (2018) Machine-Learning-Guided Mutagenesis for Directed Evolution of Fluorescent Proteins, ACS synthetic biology 7, 2014-2022.
Sandalli, C., Singh, K., Modak, M. J., Ketkar, A., Canakci, S., Demir, I., and Belduz, A. O. (2009) A new DNA polymerase I from Geobacillus caldoxylosilyticus TK4: cloning, characterization, and mutational analysis of two aromatic residues, Applied microbiology and biotechnology 84, 105-117.
Sen, S., Venkata Dasu, V. and Mandal, B. (2007) Developments in directed evolution for improving enzyme functions. Applied biochemistry and biotechnology, 143, 212-223.
Shi, C., Shen, X., Niu, S. and Ma, C. (2015) Innate Reverse Transcriptase Activity of DNA Polymerase for Isothermal RNA Direct Detection. J. Am. Chem. Soc., 137, 13804-13806.
Shroff, R., Cole, A. W., Diaz, D. J., Morrow, B. R., Donnell, I., Annapareddy, A., Gollihar, J., Ellington, A. D. and Thyer, R. (2020) Discovery of Novel Gain-of-Function Mutations Guided by Structure-Based Deep Learning. ACS synthetic biology, 9, 2927-2935.
Simon, A. J., Zhou, Y., Ramasubramani, V., Glaser, J., Pothukuchy, A., Gollihar, J., Gerberich, J. C., Leggere, J. C., Morrow, B. R., Jung, C. et al. (2019) Supercharging enables organized assembly of synthetic biomolecules. Nature chemistry, 11, 204-212.
Tanner, N. A. and Evans, T. C., Jr. (2014) Loop-mediated isothermal amplification for detection of nucleic acids. Current protocols in molecular biology, 105, Unit 15 14.
Teng, S., Srivastava, A. K., and Wang, L. (2010) Sequence feature-based prediction of protein stability changes upon amino acid substitutions, BMC genomics 11 Suppl 2, SS.
Tokuriki, N., Stricher, F., Serrano, L. and Tawfik, D. S. (2008) How Protein Stability and New Functions Trade Off. PLoS Comp. Biol., 4, e1000002.
Torng, W., and Altman, R. B. (2017) 3D deep convolutional neural networks for amino acid environment similarity analysis, BMC bioinformatics 18, 302.
Tungtur, S., Meinhardt, S. and Swint-Kruse, L. (2010) Comparing the functional roles of nonconserved sequence positions in homologous transcription repressors: implications for sequence/function analyses. Journal of molecular biology, 395, 785-802.
Vogels, C. B. F., Brackney, D., Wang, J., Kalinich, C. C., Ott, I., Kudo, E., Lu, P., Venkataraman, A., Tokuyama, M., Moore, A. J. et al. (2020) SalivaDirect: Simple and sensitive molecular diagnostic test for SARS-CoV-2 surveillance. medRxiv, 2020.2008.2003.20167791.
Wang, Y., Prosen, D. E., Mei, L., Sullivan, J. C., Finney, M. and Vander Horn, P. B. (2004) A novel strategy to engineer DNA polymerases for enhanced processivity and improved performance in vitro. Nucleic acids research, 32, 1197-1207.
Weber, D. J., Serpersu, E. H., Shortie, D. and Mildvan, A. S. (1990) Diverse interactions between the individual mutations in a double mutant at the active site of staphylococcal nuclease. Biochemistry, 29, 8632-8642.
Wu, Z., Kan, S. B. J., Lewis, R. D., Wittmann, B. J., and Arnold, F. H. (2019) Machine learning-assisted directed protein evolution with combinatorial libraries, Proceedings of the National Academy of Sciences of the United States of America 116, 8852-8858.
Yan, C., Cui, J., Huang, L., Du, B., Chen, L., Xue, G., Li, S., Zhang, W., Zhao, L., Sun, Y. et al. (2020) Rapid and visual detection of 2019 novel coronavirus (SARS-CoV-2) by a reverse transcription loop-mediated isothermal amplification assay. Clin Microbiol Infect, 26, 773-779.
Yang, H., Liu, L. and Xu, F. (2016) The promises and challenges of fusion constructs in protein biochemistry and enzymology. Appl Microbiol Biotechnol, 100, 8273-8281.
Yang, Y., Niroula, A., Shen, B., and Vihinen, M. (2016) PON-Sol: prediction of effects of amino acid substitutions on protein solubility, Bioinformatics 32, 2032-2034.
Zhang, Y., Odiwuor, N., Xiong, J., Sun, L., Nyaruaba, R. O., Wei, H. and Tanner, N. A. (2020) Rapid Molecular Detection of SARS-CoV-2 (COVID-19) Virus RNA Using Colorimetric LAMP. medRxiv, 2020.2002.2026.20028373.

1. A recombinant protein comprising, from N-terminus to C-terminus, an N-terminal stabilizer domain, optionally a first linker region, and a heterologous protein domain, wherein the N-terminal stabilizer domain comprises a sequence at least 90% identical to either SEQ ID NO: 2 or 3.

2. (canceled)

3. The recombinant protein of claim 1, wherein the N-terminal stabilizer domain has a negatively charged surface and a positively charged surface.

4. The recombinant protein of claim 3, wherein the N-terminal stabilizer domain comprises at least one substitution that enhances the positivity of the positively charged surface of the domain.

5-6. (canceled)

7. The recombinant protein of claim 1, wherein the N-terminal stabilizer domain comprises at least one substitution to a positively charged amino acid at a position corresponding to K9, A20, N31, N39, or E43 of SEQ ID NO: 2.

8-11. (canceled)

12. The recombinant protein of claim 1, wherein the N-terminal stabilizer domain comprises at least two substitutions selected from K9D, A20K, N31R, N39K, and E43K.

13-20. (canceled)

21. The recombinant protein of claim 1, wherein the heterologous protein is a nucleic acid polymerase.

22. (canceled)

23. The recombinant protein of claim 21, wherein the nucleic acid polymerase is a DNA-dependent DNA polymerase and/or an RNA-dependent DNA polymerase.

24-26. (canceled)

27. The recombinant protein of claim 21, wherein the nucleic acid polymerase is a Bst DNA polymerase, large fragment (Bst LF).

28-30. (canceled)

31. The recombinant protein of claim 27, wherein the nucleic acid polymerase comprises at least one substitution at a position corresponding to V191, S371, T493, A552, or R562 of SEQ ID NO: 4.

32-36. (canceled)

37. The recombinant protein of claim 27, wherein the nucleic acid polymerase comprises at least two substitutions selected from V191L, S371D, T493N, A552G, and R562V.

38-44. (canceled)

45. The recombinant protein of claim 21, wherein the recombinant protein comprises a sequence at least 95% identical to SEQ ID NO: 19.

46-68. (canceled)

69. The recombinant protein of claim 1, wherein the first linker region is a flexible linker or a cleavable linker.

70-71. (canceled)

72. The recombinant protein of claim 1, further comprising an augmentative protein domain positioned N-terminally relative to the N-terminal stabilizer domain.

73. The recombinant protein of claim 72, wherein the augmentative protein domain is a single-stranded DNA binding protein.

74-85. (canceled)

86. A nucleic acid encoding the recombinant protein of claim 1.

87. A host cell comprising the nucleic acid of claim 86.

88. (canceled)

89. A kit comprising the recombinant protein of claim 1.

90. (canceled)

91. A method of amplifying a target nucleic acid, the method comprising exposing a sample that may contain the target nucleic acid to a buffer solution comprising oligonucleotide primers that are capable of hybridizing to the target nucleic acid and amplifying the target nucleic acid using the recombinant protein of claim 21.

92-104. (canceled)

105. A method of diagnosing a subject with a disease, the method comprising carrying out the method of claim 91, wherein the presence of a target nucleic acid indicates the presence of a disease in the subject.

106-108. (canceled)

109. A method for expression of a protein of interest comprising expressing in a host cell a nucleic acid molecule encoding the protein of interest fused to a N-terminal stabilizer domain comprising a sequence at least 90% identical to either SEQ ID NO: 2 or 3.

110-154. (canceled)


SEQUENCE LISTING (51 sequences)

SEQ ID NO: 1 - protein, 36 aa, Artificial Sequence [sequence omitted]

SEQ ID NO: 2 - protein, 47 aa, Gallus gallus
PRGVDPSRKENHLSDEDFKAVFGMTRSAFANLPLWKQQNLKKEKGLF

SEQ ID NO: 3 - protein, 48 aa, Artificial Sequence
  Synthetic polypeptide
MPRGVDPSRKENHLSDEDFKAVFGMTRSAFANLPLWKQQNLKKEKGLF

SEQ ID NO: 4 - protein, 592 aa, Artificial Sequence
  Synthetic construct
MESPSSEEEKPLAKMAFTLADRVTEEMLADKAALVVEVVEENYHDAPIVGIAVVNHEGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWILFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQAHLLLVQHDELILEAPKEEMERLCRLVPEVMEQAVTLRVPLKVDYHYGSTWYDAK

SEQ ID NO: 5 - protein, 553 aa, Artificial Sequence
  Synthetic construct
MLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 6 - protein, 568 aa, Artificial Sequence
  Synthetic construct
MASRGSHHHHHHIEGRLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIEGLLKVVRPDTKKVHTRFNQTATATGRLSSSDPNLQNIPVRTPLGQRIRRAFIAGEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKSAMVKLFPRLEEMGARMLLQVHDELVLEAPKERAEAVARLAKEVMEGVYPLAVPLEVEVGIGEDWLSAKE

SEQ ID NO: 7 - protein, 592 aa, Artificial Sequence
  Synthetic construct
MESPSSEEEKPLAKMAFTLADRVTEEMLADKAALVVEVVEENYHDAPIVGIAVVNHEGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAGQLGTVEQRIYELAGQEFNINSPKRLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWILFAADYSQIELRVLAHIAEDDNLMEAFRRDLDIHTKTAMDIFQVSEDEVTPSMRRQAKAVNFGIVYGISDYGLAWNLNISRKEAAEFIGRYFESFPGVKRYMENIVQEAKQKGYVTTLLHRRRYLPDITSRNFNVRSFAERMAMNTPIQGSAADIIKKAMIDLNARLKEERLQAHLLLQVHDELILEAPKEEMERLCRLVPEVMEQAVTLRVPLKVDYHYGSTWYDAK

SEQ ID NO: 8 - protein, 8 aa, Artificial Sequence
  Synthetic peptide
GSGSAAAP

SEQ ID NO: 9 - protein, 12 aa, Artificial Sequence
  Synthetic peptide
SSSGSSGSSGSS

SEQ ID NO: 10 - protein, 8 aa, Artificial Sequence
  Synthetic peptide
GGSSGGSS

SEQ ID NO: 11 - protein, 8 aa, Artificial Sequence
  Synthetic peptide
SSSGSGSG

SEQ ID NO: 12 - protein, 7 aa, Artificial Sequence
  Synthetic peptide
ALALALA

SEQ ID NO: 13 - protein, 9 aa, Artificial Sequence
  Synthetic peptide
ALALALAPA

SEQ ID NO: 14 - protein, 10 aa, Artificial Sequence
  Synthetic peptide
SSSALALALA

SEQ ID NO: 15 - protein, 9 aa, Artificial Sequence
  Synthetic peptide
SGSGSGSGS

SEQ ID NO: 16 - protein, 10 aa, Artificial Sequence
  Synthetic peptide
SSSGSGSGSG

SEQ ID NO: 17 - protein, 7 aa, Artificial Sequence
  Synthetic peptide
GSSGSGS

SEQ ID NO: 18 - protein, 15 aa, Artificial Sequence
  Synthetic peptide
GGGGSGGGGSGGGGS

SEQ ID NO: 19 - protein, 644 aa, Artificial Sequence
  Synthetic construct
  INIT_MET (1)..(1)
  MISC_FEATURE (2)..(10): 8x His tag
  MISC_FEATURE (11)..(57): vHP47
  MISC_FEATURE (58)..(65): 2xGS3xAP linker
  MISC_FEATURE (66)..(644): Bst-LF
MHHHHGHHHHPRGVDPSRKENHLSDEDFKAVFGMTRSAFANLPLWKQQNLKKEKGLFGSGSAAAPKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQAHLLLQVHDELILEAPKEEMERLCRLVPEVMEQAVTLRVPLKVDYHYGSTWYDAK

SEQ ID NO: 20 - DNA, 1935 bp, Artificial Sequence
  Synthetic construct
atgcaccatcatcacggtcatcaccaccatccgcgtggtgttgacccgagccgtaaggagaaccacctgtctgacgaagacttcaaggcggtgttcggtatgacccgttctgcgttcgcgaacctgccgctgtggaaacaacagaacctgaagaaggagaaaggtctgttcggttctggaagcgcagcagcacctaagatggcattcacattggccgatcgtgtcaccgaagagatgctggcagacaaggcagccttggtcgtggaggtagttgaggagaactatcacgacgcaccgattgttggaatcgccgtggtcaatgaacatggtcgcttcttcttgcgccctgagactgcgttggccgacccacaattcgtggcctggttaggagatgaaacgaagaagaagtcaatgttcgacagcaaacgcgcagccgtagctctgaagtggaaaggaattgagctgtgtggtgtgagtttcgaccttctcttagcagcgtacttgcttgatcccgctcaaggcgtcgacgacgtggcagccgctgccaagatgaagcaatatgaagcggtgcgtccggatgaggctgtgtacgggaagggagctaaacgcgcggtgcctgatgaacccgtgcttgctgagcacttggtacgcaaggctgcggctatctgggagctggagcgtcccttcctggatgagttgcgtcgcaacgagcaggaccgcctgcttgtagagttagaacagcctcttagctctattcttgccgagatggagttcgctggtgtcaaagtagataccaagcgccttgagcaaatgggtaaggagttggctgaacaactgggcacagtggaacagcgtatctacgaactggccggtcaggagttcaacatcaacagccccaagcagctgggagtgatcctgttcgagaagttgcagctgccagtattgaagaagactaagactggctacagtacctcggctgacgtactggagaagctggctccttaccatgagatcgtggagaacatcttgcactaccgccagctgggcaagctgcagtctacctacattgagggtctgttaaaggtcgtgcgtccagacacgaagaaggtgcatacgatcttcaatcaggcgctgacccaaactggtcgtttgtcgtccacagagcccaatcttcagaatatccctattcgtcttgaggaaggccgcaagattcgccaggccttcgttccttcggaatcggactggctgatcttcgcagcagattactcacagatcgagcttcgcgtgttggcacatatcgcggaggatgacaacttaatggaggcgttccgccgcgatctggatatccatactaagaccgcgatggatatcttccaagtgtcagaagacgaggtaacaccgaacatgcgacgccaggcgaaagcggttaacttcggcatcgtctacggcatcagcgactatggcctggcccagaacttgaacatcagccgcaaggaggcagccgagttcatcgagcgctacttcgagagtttcccaggtgtgaagcgttatatggagaatatcgtacaagaggcgaagcagaaaggctacgtgaccacgctgttacaccgtcgtcgctaccttcctgatatcactagccgtaacttcaatgtacgttccttcgccgaacgcatggcgatgaatacccccatccaggggtcagctgcagatatcatcaagaaagctatgatcgacttaaacgctcgtctgaaggaagaacgcttacaggcgcacctcttactgcaagtccatgacgaattgatccttgaggcgcccaaggaagagatggagcgtctttgccgtctggtgccggaagtaatggaacaggccgtcacgctgcgcgtacctctgaaagtcgattaccactacggctccacctggtatgacgccaagtaa

SEQ ID NO: 21 - protein, 47 aa, Artificial Sequence
  Synthetic polypeptide
PRGVDPSRKEDHLSDEDFQAVFGMNRSAFSNLPLWKQQKLKKDKGLF

SEQ ID NO: 22 - DNA, 18 bp, Artificial Sequence
  Synthetic polypeptide
gccacccagaagactgtg

SEQ ID NO: 23 - DNA, 20 bp, Artificial Sequence
  Synthetic polynucleotide
tggcaggtttttctagacgg

SEQ ID NO: 24 - DNA, 39 bp, Artificial Sequence
  Synthetic polynucleotide
cgccagtagaggcagggatgagggaaactgtggcgtgat

SEQ ID NO: 25 - DNA, 40 bp, Artificial Sequence
  Synthetic polynucleotide
ggtcatccctgagctgaacggtcaggtccaccactgacac

SEQ ID NO: 26 - DNA, 22 bp, Artificial Sequence
  Synthetic polynucleotide
tgttctggagagccccgcggcc

SEQ ID NO: 27 - DNA, 36 bp, Artificial Sequence
  Synthetic polynucleotide
ctcactggcatggccttccgtgtccccactgccaac

SEQ ID NO: 28 - DNA, 25 bp, Artificial Sequence
  Synthetic polynucleotide
ggacacggaaggccatgccagtgag

SEQ ID NO: 29 - DNA, 482 bp, Artificial Sequence
  Synthetic polynucleotide
ctagtaacggccgccagtgtgctggaattcccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggaattctgcagatatccatcacactggcggccgctcgagc

SEQ ID NO: 30 - DNA, 19 bp, Artificial Sequence
  Synthetic polynucleotide
accgaagagctaccagacg

SEQ ID NO: 31 - DNA, 20 bp, Artificial Sequence
  Synthetic polynucleotide
tgcagcattgttagcaggat

SEQ ID NO: 32 - DNA, 40 bp, Artificial Sequence
  Synthetic polynucleotide
tctggcccagttcctaggtagttcgtggtggtgacggtaa

SEQ ID NO: 33 - DNA, 40 bp, Artificial Sequence
  Synthetic polynucleotide
agacggcatcatatgggttgcacgggtgccaatgtgatct

SEQ ID NO: 34 - DNA, 21 bp, Artificial Sequence
  Synthetic polynucleotide
actgagggagccttgaataca

SEQ ID NO: 35 - DNA, 35 bp, Artificial Sequence
  Synthetic polynucleotide
ccgaatgaaagatctcagtccaagatggtatttct

SEQ ID NO: 36 - DNA, 25 bp, Artificial Sequence
  Synthetic polynucleotide
tcttggactgagatctttcattcgg

SEQ ID NO: 37 - DNA, 21 bp, Artificial Sequence
  Synthetic polynucleotide
tccagatgaggatgaagaaga

SEQ ID NO: 38 - DNA, 21 bp, Artificial Sequence
  Synthetic polynucleotide
agtctgaacaactggtgtaag

SEQ ID NO: 39 - DNA, 41 bp, Artificial Sequence
  Synthetic polynucleotide
agagcagcagaagtggcacaggtgattgtgaagaagaagag

SEQ ID NO: 40 - DNA, 40 bp, Artificial Sequence
  Synthetic polynucleotide
tcaacctgaagaagagcaagaactgattgtcctcactgcc

SEQ ID NO: 41 - DNA, 22 bp, Artificial Sequence
  Synthetic polynucleotide
ctcatattgagttgatggctca

SEQ ID NO: 42 - DNA, 22 bp, Artificial Sequence
  Synthetic polynucleotide
acaaactgttggtcaacaagac

SEQ ID NO: 43 - DNA, 47 bp, Artificial Sequence
  Synthetic polynucleotide
gtatggtactgaagatgattaccaaggtaaacctttggaatttggac

SEQ ID NO: 44 - DNA, 36 bp, Artificial Sequence
  Synthetic polynucleotide
gtccaaattccaaaggtttaccttggtaatcatctc

SEQ ID NO: 45 - DNA, 18 bp, Artificial Sequence
  Synthetic polynucleotide
tgcttcagtcagctgatg

SEQ ID NO: 46 - DNA, 22 bp, Artificial Sequence
  Synthetic polynucleotide
ttaaattgtcatcttcgtcctt

SEQ ID NO: 47 - DNA, 41 bp, Artificial Sequence
  Synthetic polynucleotide
tcagtactagtgcctgtgcccacaatcgtttttaaacgggt

SEQ ID NO: 48 - DNA, 43 bp, Artificial Sequence
  Synthetic polynucleotide
tcgtatacagggcttttgacatctatcttggaagcgacaacaa

SEQ ID NO: 49 - DNA, 22 bp, Artificial Sequence
  Synthetic polynucleotide
gtagctggttttgctaaattcc

SEQ ID NO: 50 - DNA, 33 bp, Artificial Sequence
  Synthetic polynucleotide
acaggtgtaagtgcagcccgtcttacaccgtgc

SEQ ID NO: 51 - DNA, 22 bp, Artificial Sequence
  Synthetic polynucleotide
gacgggctgcacttacacctgt